(19) United States
(12) Patent Application Publication
(10) Pub. No.: US 2022/0265723 A1
(43) Pub. Date: Aug. 25, 2022

(54) NON-VIRAL MODIFICATION OF MESENCHYMAL STEM CELLS

(71) Applicant: NATIONAL UNIVERSITY OF SINGAPORE, Singapore (SG)

(72) Inventors: Heng-Phon Too, Singapore (SG); Yoon Khei Ho, Singapore (SG); Xue En Geraldine Tu, Singapore (SG)

(21) Appl. No.: 17/436,338

(22) PCT Filed: Mar. 6, 2020
(86) PCT No.: PCT/IB2020/051983

§ 371 Date: Sep. 3, 2021

(30) Foreign Application Priority Data
Mar. 6, 2019 (SG) 10201902002S

(51) Int. Cl. A61K 35/28; C12N 15/85; C12N 9/78; C12N 9/10; A61K 38/50; A61K 38/45; A61K 31/513; A61K 31/522; A61P 35/00; A61K 9/00

(52) U.S. Cl. CPC A61K 35/28; C12N 15/85; C12N 9/78; C12Y 305/04001; C12N 9/1077; C12Y 204/02009; A61K 38/50; A61K 38/45; A61K 31/513; A61K 31/522; A61P 35/00; A61K 9/0019

(57) ABSTRACT

Described herein are methods for transfecting mesenchymal stem cells (MSCs) with a nucleic acid construct using a cationic polymer, a first reagent capable of redirecting endocytosed nucleic acids from intracellular acidic compartments, and a second agent capable of stabilizing a microtubular network of the MSCs. The methods are free of virus-based transfection vehicle materials and the transfected MSCs have substantially unchanged multipotent phenotype. In certain embodiments, the transfected MSCs express functional genes comprising suicide gene, such as cytosine deaminase or uracil phosphoribosyltransferase. Also described are methods for the treatment of diseases, such as cancer, using such transfected cells in combination with 5FC, 5FU, GCV, as well as kits and composition relating thereof.

### FIELD OF INVENTION

The present invention relates generally to non-viral modification of mesenchymal stem cells. More specifically, the present invention relates to non-viral modification of mesenchymal stem cells (MSCs) for therapeutic uses such as cancer treatment.

### BACKGROUND

Currently, there are >700 clinical trials using mesenchymal stem cells (MSCs) registered on the National Institutes of Health clinical trials database. While MSC-based treatments are considered safe [2], preclinical and clinical data have shown moderate effect at best and often ineffectiveness [3-5]. To overcome this impasse, the emerging trend is to genetically modify the MSCs. In the United Kingdom alone, 37% of the registered trials use genetically modified cells, 90% of which use viral carriers for gene delivery to MSCs [6]. Clinical trials with modified MSCs to produce cytosine deaminase (CD) for gene-directed enzyme prodrug therapy (GDEPT) are underway [7]. In view of the inherent safety and production issues with viruses [7, 8], highly efficient modification of MSCs using non-viral methods is desirable but poses significant challenges.

Many preclinical studies and clinical trials [28-31] have exploited viral vectors as efficient gene delivery vehicles in the modification of MSCs. While viral gene delivery is highly efficient, there are drawbacks which may include random integration of virus vector into the host genome, which may interrupt essential gene expression and cellular processes. Even with non-integrating viral vectors, safety risks of viral transduction may arise due to possible presentation of viral antigens on transduced cells that could potentially activate an immune response in vivo following transplantation. Production of viral vectors is both labour intensive and technically demanding, thus posing a challenge to scale up with increasing number of transgenes. Furthermore, it is worthy to note that cells infected with viral vectors typically have low copy numbers (<10 copies/cell). While viruses enabled sustained expression of transgene [32], cells infected with virus typically have low copy numbers (<10 copies/cell) [33, 34]. On the other hand, studies has shown that increased DNA copy numbers can be delivered into individual cells with non-viral methods [35, 36] hence increasing payload in delivering therapeutic agents, however these typically suffer from low transfection efficiency (often about 0-35%). Production of clinical grade virus is laborious and typically involves generation as well as certification of a master cell bank of stable producer lines, thus incurring high cost in gene-cell therapeutics [37-39].

Non-viral methods often suffer from drawbacks preventing clinical use. Non-viral methods, for example cationic polymers, liposomes, electroporation and others, typically suffer from poor efficiency in modifying MSCs at scales relevant to clinical treatment. In addition, non-viral methods such as electroporation may have a low cell viability, hindering use on large scale.

Transient transfection is an approach to obtain high payload per cell rapidly, avoiding antibiotic selection and weeks of process work that may cause cell senescence [17] and reduce tumour tropism [18] as well as safety concerns with viral induced MSC transformation [19]. Although certain non-viral methods have advantages over viral vectors for the ease of production, low cost and safety profiles [20], the lack of wide adoption for MSC modification is mainly due to the low efficiency of transfection (0-35%) [21, 22]. While high copies of DNA may be delivered into the cells, the expression of transgene often remains low. The low expression of the transgene with certain non-viral methods may be due to the accumulation of plasmid DNA in non-productive intracellular compartments, rendering low availability of plasmid for gene transcription.

Additional, alternative, and/or improved methods for the transfection of MSCs is desired.

### SUMMARY OF INVENTION

Stem cells modified to express therapeutic genes, or other genes of interest, are desirable for a number of different therapeutic and non-therapeutic applications. Traditionally, in the field of prodrug gene therapy, virus-based gene modification approaches have been the favoured approach for modifying stem cells such as MSCs in preclinical and clinical studies, since non-viral approaches have generally provided poor transfection efficiency. However, virus-based gene modification in such applications has inherent safety risk, production of clinical grade virus can be laborious, and the number of gene copies which may be introduced per cell through viral methods is generally low (often <10 copies per cell). Furthermore, achieving gene modification of stem cells such as MSCs, either virally or non-virally, without causing undesirable changes to phenotype (i.e. multipotency, immunophenotype, tropism, etc.) of the resultant cells is another challenge facing the field.

As described in detail herein, the present inventors have now developed methods for transfecting mesenchymal stem cells with a nucleic acid construct from which one or more functional genes are expressed, which are non-viral and which in certain embodiments may provide high transfection efficiency, high copy number per cell, high cell viability, transient expression for extended duration, and/or a substantially unchanged multipotent phenotype. In certain embodiments, such methods may be scalable and/or suitable for large scale clinical production of modified mesenchymal stem cells. Also described in detail herein are transfected mesenchymal stem cells and populations of mesenchymal stem cells, uses thereof, methods for the treatment of diseases or disorders such as cancer using such transfected stem cells, and kits and compositions relating thereto.

In one embodiment, there is provided herein a mesenchymal stem cell (MSC) transfected with a nucleic acid construct from which one or more functional genes are expressed, the MSC having a multipotent phenotype which is substantially unchanged by the transfection of the nucleic acid construct, and the MSC being free of virus-based transfection vehicle materials.

In another embodiment, there is provided herein a plurality of mesenchymal stem cells (MSCs), wherein at least about 60% of the MSCs are transfected with a nucleic acid construct from which one or more functional genes are expressed, the transfected MSCs having a multipotent phenotype which is substantially unchanged by the transfection of the nucleic acid construct, and the MSCs being free of virus-based transfection vehicle materials.

In another embodiment of the plurality of MSCs, at least about 65%, at least about 70%, at least about 75%, at least about 80%, at least about 85%, at least about 90%, or at least about 95% of the MSCs may be transfected with the nucleic acid construct and express the one or more functional genes. In further embodiments, a cell viability of the plurality of MSCs may be at least about 70%, at least about 75%, at least about 80%, or at least about 85%.

In another embodiment, of any of the transfected MSC or MSCs herein, the MSC or MSCs may be each transfected with an average of at least about 1000, at least about 2000, at least about 3000, at least about 4000, at least about 5000, at least about 6000, at least about 7000, at least about 8000, at least about 9000, or at least about 10000 copies of the nucleic acid construct. In another embodiment, the one or more functional genes may be transiently expressed in the transfected MSC cell or cells. In another embodiment, the MSC or MSCs may be derived from cord blood, neonatal birth-associated tissue, Wharton's jelly, umbilical cord, cord lining, placenta, or other source of MSC cells. In another embodiment, the MSC or MSCs may be adipose tissue-derived MSC (AT-MSC), bone marrow-derived MSC (BM-MSC), or umbilical cord-derived MSC (UC-MSC). In another embodiment, the MSCs may be sourced from human, canine, feline, equine, or other species. In another embodiment, the nucleic acid construct may comprise a CpG-free expression plasmid or other CpG-free expression construct, a scaffold/matrix attachment region (S/MAR), an episomal vector, or an EBNA-1 containing construct.

In another embodiment, of any of the transfected MSC or MSCs herein, the MSC or MSCs may transiently express the one or more functional genes for at least about 7, at least about 8, at least about 9, at least about 10, at least about 11, at least about 12, at least about 13, at least about 14, at least about 15, at least about 16, or at least about 17 days following transfection. In another embodiment, the one or more functional genes may comprise a suicide gene. In another embodiment, the one or more functional genes may comprise Cytosine Deaminase (CDy). In another embodiment, the one or more functional genes may comprise uracil phosphoribosyltransferase (UPRT). In another embodiment, the one or more functional genes may comprise both CDy and UPRT. In another embodiment, the CDy and UPRT may be expressed as a fused construct. In another embodiment, the one or more functional genes may comprise a fluorescent protein. In another embodiment, the fluorescent protein may comprise green fluorescent protein (GFP). In another embodiment, the one or more functional genes may comprise CDy, UPRT, and GFP. In another embodiment, the CDy, UPRT, and GFP may be expressed as a fused construct. In another embodiment, one or more functional genes may comprise herpes simplex virus-1 thymidine kinase (HSV-TK) or another thymidine kinase. In another embodiment, the one or more functional genes may comprise one or more cancer therapy genes, or one or more functional genes which are not related to cancer therapy.

In another embodiment, of any of the transfected MSC or MSCs herein, the transfected MSC or MSCs may be transfected with the nucleic acid construct using a cationic polymer, a first agent capable of redirecting endocytosed nucleic acids from intracellular acidic compartments, and a second agent capable of stabilizing a microtubular network of the MSC or MSCs. In another embodiment, the cationic polymer may comprise linear or branched polyethylenimine (PEI), poly(amidoamine) PAMAM, or another cationic polymer, or any combination thereof. In another embodiment, the cationic polymer may comprise linear polyethylenimine (LPEI). In another embodiment, the first agent may comprise 1,2-Dioleoyl-sn-glycero-3-phosphoethanolamine (DOPE)/cholesteryl hemisuccinate (CHEMS) (DOPE/CHEMS), 1,2-dipalmitoyl-sn-glycero-3-phosphocholine (DPPC) or another fusogenic lipid, or any combinations thereof. In another embodiment, the second agent may comprise a histone deactylase inhibitor (HDACi), such as a histone deactylase 6 inhibitor (HDAC6i). In another embodiment, the second agent may comprise SAHA (Vorinostat).

In another embodiment or any of the MSC or MSCs herein, the phenotype may include tumor and/or cancer tropism properties of the MSC or MSCs. In another embodiment, the genetically engineered MSC or MSCs of any embodiments described herein may be sensitive to treatment with 5-fluorocytosine (5FC) or ganciclovir (GCV). One or more embodiments of the MSC or MSCs may convert: a) 5FC to 5-fluorouracil (5FU), 5-fluorouridine monophosphate (FUMP), or both; b) ganciclovir to ganciclovir monophosphate; or c) a combination of a) and b). In another embodiment the phenotype may comprise an immunophenotype in which the expression of CD surface markers may be substantially unchanged after transfection.

In another embodiment of any of the MSC or MSCs described herein, the transfected MSC or MSCs may be plastic-adherent, may express CD105, CD73, and CD90 (>95%), may lack expression of CD45, CD34, CD14, and HLA-DR surface molecules (<2%), and may be capable of differentiating into osteoblasts, adipocytes, and chondroblasts in vitro, satisfying the immunophenotype criteria defined by the International Society for Cellular Therapy (ISCT). In another embodiment, the transfected MSC or MSCs may be undifferentiated.

In another embodiment of any of the MSC or MSCs described herein, the MSC or MSCs may be in a cryopreserved state.

In another embodiment, the MSC or MSCs may be for use in treating cancer. In certain embodiments, the cancer may comprise lymphoma, clear cell carcinoma, glioblastoma, temozolomide resistant glioblastoma, perianal carcinoma, oral melanoma, thyroid carcinoma, soft tissue carcinoma, cancer ulceration, nasal tumor, or gastrointestinal cancer, or any combination thereof. In another embodiment, the MSC or MSCs may be for use in combination with 5FC, 5FU, GCV, or any combination thereof.

In another embodiment, there is provided herein a method for transfecting mesenchymal stem cells (MSCs) with a nucleic acid construct from which one or more functional genes are expressed, the method comprising: exposing the MSCs to a transfection mixture comprising the nucleic acid construct which is complexed with a cationic polymer; exposing the MSCs to a first agent capable of redirecting endocytosed nucleic acids from intracellular acidic compartments and a second agent capable of stabilizing a microtubular network of the MSCs; and incubating the MSCs; thereby providing MSCs transfected with the nucleic acid construct.

In another embodiment of and of the method or methods described herein the MSCs may not be centrifuged during exposure to the transfection mixture, to the first agent and second agent, during incubation, or any combination thereof. In another embodiment the step of incubating the MSCs may comprise gentle mixing without centrifugation. In another embodiment the step of incubating the MSCs may comprise incubating the MSCs for at least about 2 hours. In another embodiment the step of incubating the MSCs may comprise incubating the MSCs for about 2 hours to about 48 hours. In another embodiment the step of incubating the MSCs may comprise incubating the MSCs for about 3 hours to about 24 hours, or for about 4 hours to about 18 hours.

In another embodiment of any of the method or methods described herein, the cationic polymer may comprise a cationic polymer which has been identified as having low cytotoxicity against the MSCs. In another embodiment the cationic polymer may have a size of about 5 kDa to about 200 kDa. In another embodiment the cationic polymer may comprise linear or branched polyethylenimine (PEI), poly(amidoamine) PAMAM, or another cationic polymer, or any combinations thereof. In another embodiment the cationic polymer may comprise linear polyethylenimine (LPEI).

In another embodiment of any of the method or methods described herein, the first agent may comprise 1,2-Dioleoyl-sn-glycero-3-phosphoethanolamine (DOPE)/cholesteryl hemisuccinate (CHEMS) (DOPE/CHEMS), 1,2-dipalmitoyl-sn-glycero-3-phosphocholine (DPPC) or another fusogenic lipid, or any combinations thereof. In another embodiment the second agent may comprise a histone deacetylase inhibitor (HDACi), such as a histone deacetylase 6 inhibitor (HDAC6i). In another embodiment the second agent may comprise SAHA (Vorinostat).

In another embodiment of any of the method or methods described herein, the step of exposing the MSCs to the transfection mixture may comprise complexing the nucleic acid construct with the cationic polymer so as to provide the transfection mixture comprising complexed nucleic acid construct, and adding the transfection mixture to the MSCs. In another embodiment, the step of exposing the MSCs to the transfection mixture may comprise adding the transfection mixture to the MSCs and incubating the MSCs with the transfection mixture. In another embodiment, the step of exposing the MSCs to the first and second agents may comprise replacing the transfection mixture with cell culture media supplemented with the first agent and second agent. In another embodiment, the step of exposing the MSCs to the transfection mixture may comprise removing a culture media from the MSCs and replacing the culture media with the transfection mixture. In another embodiment the step of exposing the MSC to the transfection mixture may comprise incubating the MSCs with the transfection mixture under mild centrifugation. In another embodiment the mild centrifugation may comprise about 200 g for about 5 minutes.

In another embodiment of any of the method or methods described herein, the cell culture media may comprise complete media. In another embodiment the MSCs may be at about 60% confluency, and the MSCs may be seeded for about 24 hours prior to exposure to the transfection mixture. In another embodiment, the transfection mixture may comprise the complexed nucleic acid construct in serum free DMEM, or in fresh culture media.

In another embodiment of any of the method or methods described herein, the amount of nucleic acid construct in the transfection mixture to which the MSCs are exposed may be between about 200 to about 500 ng per 1.9 cm² surface area. In another embodiment, the amount of nucleic acid construct in the transfection mixture to which the MSCs are exposed may be between about 250 to about 400 ng per 1.9 cm² surface area. In another embodiment, the amount of nucleic acid construct in the transfection mixture to which the MSCs are exposed may be between about 300 to about 350 ng per 1.9 cm² surface area. In another embodiment, a ratio of cationic polymer to nucleic acid construct may be about 1 μg to about 30 μg cationic polymer per 1 μg of nucleic acid construct in the transfection mixture. In another embodiment, the transfected MSCs may be each transfected with an average of at least about 1000, at least about 2000, at least about 3000, at least about 4000, at least about 5000, at least about 6000, at least about 7000, at least about 8000, at least about 9000, or at least about 10000 copies of the nucleic acid construct. In another embodiment, the nucleic acid construct may comprise a CpG-free expression plasmid or other CpG-free expression construct, a scaffold/matrix attachment region (S/MAR), an episomal vector, or an EBNA-1 containing construct.

In another embodiment of any of the method or methods described herein, the one or more functional genes may comprise a suicide gene. In another embodiment, the one or more functional genes may comprise Cytosine Deaminase (CDy) and/or thymidine kinase (TK). In another embodiment, the one or more functional genes may comprise uracil phosphoribosyltransferase (UPRT). In another embodiment, the one or more functional genes may comprise both CDy and UPRT. In another embodiment, the CDy and UPRT may be expressed as a fused construct. In another embodiment, the one or more functional genes may comprise a fluorescent protein. In another embodiment, the fluorescent protein may comprise green fluorescent protein (GFP). In another embodiment, the one or more functional genes may comprise CDy, UPRT, and GFP. In another embodiment, the CDy, UPRT, and GFP may be expressed as a fused construct. In another embodiment, the one or more functional genes may comprise herpes simplex virus-1 thymidine kinase (HSV-TK) or another thymidine kinase. In another embodiment, the one or more functional genes may comprise one or more cancer therapy genes, or one or more functional genes which are not related to cancer therapy. In another embodiment, the one or more functional genes may be transiently expressed in the transfected MSCs. In another embodiment, the MSCs may transiently express the one or more functional genes for at least about 7, at least about 8, at least about 9, at least about 10, at least about 11, at least about 12, at least about 13, at least about 14, at least about 15, at least about 16, or at least about 17 days following transfection. In another embodiment, the one or more functional genes may comprise a fluorescent protein, and the method may further comprise a step of isolating, selecting, or purifying the transfected MSCs using cell sorting or FACS.

In another embodiment of any of the method or methods described herein, a multipotent phenotype of the transfected MSCs may be substantially unchanged by the transfection. For example, without wishing to be limiting, the multipotent phenotype may include differentiation potential such that the modified cells are able to differentiate to osteogenic, adipogenic and/or chondrogenic lineage, comparable to the native MSCs. In another embodiment, the multipotent phenotype may comprise tumor and/or cancer tropism properties of the MSC. In another embodiment, the multipotent phenotype may comprise an immunophenotype in which the expression of CD surface markers is substantially unchanged after transfection. In another embodiment, the transfected MSCs may be undifferentiated.

In another embodiment of any of the method or methods described herein, the transfected MSCs may be plastic-adherent, may express CD105, CD73, and CD90 (>95%), may lack expression of CD45, CD34, CD14, and HLA-DR surface molecules (<2%), and may be capable of differentiating into osteoblasts, adipocytes, and chondroblasts in vitro, satisfying the immunophenotype criteria defined by the International Society for Cellular Therapy (ISCT).

In another embodiment of any of the method or methods described herein, at least about 60%, at least about 65%, at least about 70%, at least about 75%, at least about 80%, at least about 85%, at least about 90%, or at least about 95% of the MSCs may be transfected with the nucleic acid construct and express the one or more functional genes. In another embodiment, a cell viability of the transfected MSCs may be at least about 70%, at least about 75%, at least about 80%, or at least about 85%.

In another embodiment of any of the method or methods described herein, the method may be free of virus-based transfection vehicle materials.

In another embodiment of any of the method or methods described herein, the MSCs may be derived from cord blood, neonatal birth-associated tissue, Wharton's jelly, umbilical cord, cord lining, placenta, or other source of MSC cells. In another embodiment, the MSCs may be adipose tissue-derived MSC (AT-MSC), bone marrow-derived MSC (BM-MSC), or umbilical cord-derived MSC (UC-MSC). In another embodiment, the MSC or MSCs may be sourced from human, canine, feline, equine, or other species.

In another embodiment of any of the method or methods described herein, the resultant MSCs may be sensitive to treatment with 5-fluorocytosine (5FC) or ganciclovir (GCV) or both. In another embodiment, the resultant MSC may convert: a) 5FC to 5-fluorouridine (5FU), 5-fluorouridine monophosphate (FUMP) or both; b) ganciclovir to ganciclovir monophosphate; or c) a combination of a) and b).

In another embodiment of any of the method or methods described herein, the method may comprise a step of culturing the MSCs in a growth medium, such as a fresh growth medium, before the step of exposing the MSCs to the transfection mixture. In another embodiment the step of exposing the MSCs to the transfection mixture may comprise adding the transfection mixture to the MSCs without removing the growth medium from the MSCs, and centrifugation is not performed during the steps of exposing and incubating. In another embodiment, the step of exposing the MSCs to the first agent and the second agent may comprise adding the first and second agent to the MSCs simultaneously, sequentially, or in combination with the transfection mixture. In another embodiment the first and second agent may be added to the MSCs simultaneously with addition of the transfection mixture to the MSCs, or the first and second agent may be mixed with the transfection mixture and added to the MSCs. In another embodiment the first and second agent may be added to the MSCs shortly after the transfection mixture is added to the MSCs. In another embodiment the transfection mixture may not be removed before the first and second agents are added to the MSCs. In another embodiment a duration of exposure of the MSCs to the transfection mixture may overlap with a duration of exposure of the MSCs to the first and second agents. In another embodiment the transfection mixture may not be removed before the first and second agents are added to the MSCs.

In another embodiment of any of the method or methods described herein, the method may further comprise a step of cryopreserving the transfected mesenchymal stem cells (MSCs) for storage. In another embodiment, the method may further comprise a step of thawing the cryopreserved transfected mesenchymal stem cells in preparation for use thereof.

In another embodiment of any of the method or methods described herein, the transfected MSCs are MSCs may be as defined by any of the MSC or MSCs embodiments described herein. In another embodiment, one or more embodiments of an MSC, or plurality of MSCs, may be produced by any of the method or methods as described herein.

In another embodiment, there is provided herein an MSC, or a plurality of MSCs, produced by any of the methods as described herein.

In another embodiment, there is provided herein a use of any of the MSC or MSCs as defined herein for treating cancer in a subject in need thereof. In certain embodiments, by way of non-limiting example, the cancer may comprise lymphoma, clear cell carcinoma, glioblastoma, temozolomide resistant glioblastoma, perianal carcinoma, oral melanoma, thyroid carcinoma, soft tissue carcinoma, cancer ulceration, nasal tumor, or gastrointestinal cancer. In another embodiment the MSC or MSCs may be for use in combination with 5FC, 5FU, GCV, or any combination thereof. In another embodiment the MSC or MSCs may be for use in the manufacture of a medicament for the treatment of cancer. In another embodiment the MSC or MSCs may be for use in combination with 5FC, 5FU, GCV, or any combination thereof.

In another embodiment there is provided herein a method for treating cancer in a subject in need thereof, wherein said method may comprise: administering any of the MSC or MSCs as defined herein to a region in proximity with a cancer cell of the subject, wherein the one or more functional genes in the MSC or MSCs may contribute to an anticancer effect on the cancer cell.

In certain embodiments of any of the method or methods for treating cancer described herein, the cancer may comprise lymphoma, clear cell carcinoma, glioblastoma, temozolomide resistant glioblastoma, perianal carcinoma, oral melanoma, thyroid carcinoma, soft tissue carcinoma, cancer ulceration, nasal tumor, or gastrointestinal cancer, for example.

In another embodiment of any of the method or methods for treating cancer described herein, the MSC or MSCs may be administered simultaneously, sequentially, or in combination with 5FC, 5FU, GCV, or any combination thereof. In another embodiment the one or more functional genes may comprise Cytosine Deaminase (CDy), thymidine kinase (TK), or both. In another embodiment the one or more functional genes may comprise uracil phosphoribosyltransferase (UPRT). In another embodiment the one or more functional genes may comprise both CDy and UPRT. In another embodiment the CDy and UPRT may be expressed in the MSC or MSCs as a fused construct. In another embodiment, the MSC or MSCs may transiently express the one or more functional genes for at least about 7, at least about 8, at least about 9, at least about 10, at least about 11, at least about 12, at least about 13, at least about 14, at least about 15, at least about 16, or at least about 17 days following transfection.

In another embodiment, any of the method or methods for treating cancer described herein may further comprise a step of administering 5FC, 5FU, ganciclovir, or any combination thereof, to the subject such that the MSC or MSCs are exposed to the 5FC, 5FU, ganciclovir or combination thereof.

In another embodiment, any of the method or methods for treating cancer described herein may further comprise a step of producing the MSC or MSCs according to any of the method or methods as defined in any embodiment described herein prior to the step of administering the MSC or MSCs.

In another embodiment there is provided herein a composition comprising the engineered MSC or MSCs of any embodiment described herein, and at least one of a pharmaceutically acceptable carrier, diluent, excipient, cell media, or buffer.

In another embodiment there is provided herein a theranostic agent comprising any of the MSC or MSCs of any embodiment described herein.

In another embodiment there is provided herein a kit for transfecting a mesenchymal stem cell (MSC) with a nucleic acid construct from which one or more functional genes are transiently expressed. In an embodiment the kit may comprise one or more of: an MSC; a nucleic acid construct designed for transient expression of one or more functional genes; a cell culture media; a cationic polymer; a first agent capable of redirecting endocytosed nucleic acids from intracellular acidic compartments; a second agent capable of stabilizing a microtubular network of the MSC; instructions for performing a method as described in any embodiment herein; 5FC; GCV; and/or 5FU. In certain embodiments, the kit may comprise a cryopreservation buffer or agent, a thawing buffer or agent, or both. In certain non limiting embodiments, cryopreservation buffers or solutions can be used, such as cryostor10 (Biolife Solutions USA). In further exemplary embodiments, thawed engineered MSCs can be stored in a hypothermic solution such as Hypothermosol (Biolife Solutions USA). Other examples may also be used as will be apparent to the skilled person in the art.

In another embodiment of any of the kit or kits herein, the MSC may be derived from cord blood, neonatal birth-associated tissue, Wharton's jelly, umbilical cord, cord lining, placenta, or other source of MSC cells. In another embodiment the MSC may be an adipose tissue-derived MSC (AT-MSC), bone marrow-derived MSC (BM-MSC), or umbilical cord-derived MSC (UC-MSC). In another embodiment, the MSCs may be sourced from human, canine, feline, equine, or other species. In another embodiment the nucleic acid construct may comprise a CpG-free expression plasmid or other CpG-free expression construct, a scaffold/matrix attachment region (S/MAR), an episomal vector, or an EBNA-1 containing construct. In another embodiment the cationic polymer may comprise linear or branched polyethylenimine (PEI), poly(amidoamine) PAMAM, or another cationic polymer, or any combinations thereof. In another embodiment the cationic polymer may comprise linear polyethylenimine (LPEI). In another embodiment the first agent may comprise one or more of DOPC, DPPC, or another fusogenic lipid. In another embodiment the first agent may comprise 1,2-Dioleoyl-sn-glycero-3-phosphoethanolamine (DOPE)/cholesteryl hemisuccinate (CHEMS) (DOPE/CHEMS), 1,2-dipalmitoyl-sn-glycero-3-phosphocholine (DPPC) or another fusogenic lipid, or any combinations thereof. In another embodiment the second agent may comprise a histone deactylase inhibitor (HDACi) such as a histone deacetylase 6 inhibitor (HDAC6i). In another embodiment the second agent may comprise SAHA (Vorinostat). In another embodiment the one or more functional genes may comprise a suicide gene. In another embodiment the one or more functional genes may comprise Cytosine Deaminase (CDy) or thymidine kinase (TK). In another embodiment the one or more functional genes may comprise uracil phosphoribosyltransferase (UPRT). In another embodiment the one or more functional genes may comprise both CDy and UPRT. In another embodiment, the CDy and UPRT may be expressed as a fused construct. In another embodiment the one or more functional genes may comprise a fluorescent protein. In another embodiment the fluorescent protein may comprise green fluorescent protein (GFP). In another embodiment the one or more functional genes may comprise CDy, UPRT, and GFP. In another embodiment the CDy, UPRT, and GFP may be expressed as a fused construct. In another embodiment the one or more functional genes may comprise herpes simplex virus-1 thymidine kinase (HSV-TK). In another embodiment, the one or more functional genes may comprise one or more cancer therapy genes, or one or more functional genes which are not related to cancer therapy. In another embodiment the cationic polymer may comprise a cationic polymer which has been identified as having low cytotoxicity against the MSCs. In another embodiment the cationic polymer may have a size of about 5 kDa to about 200 kDa. In another embodiment a ratio of cationic polymer to nucleic acid construct in one or more embodiments of the kit may be about 1 μg to about 30 μg cationic polymer per 1 μg of nucleic acid construct.

In another embodiment of any of the kit or kits herein, the kit may be for preparing an MSC-based anti-cancer agent. In another embodiment of any of the kit or kits herein, the kit may comprise instructions and/or apparatus for performing any of the method or methods as defined in any one of the embodiments described herein.

In another embodiment there is provided herein a kit for transfecting a mesenchymal stem cell (MSC) with a nucleic acid construct from which one or more functional genes are transiently expressed, wherein the kit may comprise any one or more of: an MSC; a nucleic acid construct designed for transient expression of one or more functional genes; a cell culture media; a cationic polymer; a first agent capable of redirecting endocytosed nucleic acids from intracellular acidic compartments; a second agent capable of stabilizing a microtubular network of the MSC; instructions for performing any of the method or methods described herein; 5FC; GCV; and/or 5FU. In certain embodiments, the kit may comprise a cryopreservation buffer or agent, a thawing buffer or agent, or both, as described above

In another embodiment there is provided herein a method for transfecting mesenchymal stem cells (MSCs) with a nucleic acid construct from which one or more functional genes are expressed, the method comprising: culturing the MSCs in a growth medium; adding a transfection mixture comprising the nucleic acid construct which is complexed with a cationic polymer to the MSCs without removing the growth medium from the MSCs; adding a first agent capable of redirecting endocytosed nucleic acids from intracellular acidic compartments and a second agent capable of stabilizing a microtubular network of the MSCs to the MSCs; and incubating the MSCs while in contact with all of the transfection mixture, the first agent, and the second agent for an incubation period; wherein the first and second agents are added to the MSCs simultaneously with the addition of the transfection mixture, sequentially with the addition of the transfection mixture, or in combination with the transfection mixture; and wherein the MSCs are not centrifuged between the adding of the transfection mixture and expiry of the incubation period; thereby providing MSCs transfected with the nucleic acid construct.

In another embodiment of the method or methods for transfecting mesenchymal stem cells (MSCs) with a nucleic acid construct from which one or more functional genes are expressed as described herein, the incubation period may be at least about 2 hours. In another embodiment, the incubation period may be about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, or about 36 hours, or more.

In another embodiment, there is provided herein an MSC cell, or plurality of MSC cells, produced by any of the method or methods for transfecting mesenchymal stem cells (MSCs) with a nucleic acid construct from which one or more functional genes are expressed as described herein. In another embodiment, there is provided herein a use of any of the MSC cell, or plurality of MSC cells, described herein for treating cancer in a subject in need thereof, or for the manufacture of a medicament for the treatment of cancer.

Methods for treating cancer in a subject in need thereof are described herein. In certain embodiments of such methods may comprise administering any of the MSC or MSCs as defined in one or more embodiments described herein to a region in proximity with a cancer cell of the subject, wherein the one or more functional genes in the MSC or MSCs contribute to an anticancer effect on the cancer cell.

In certain embodiments, the cancer may comprise lymphoma, clear cell carcinoma, glioblastoma, temozolomide resistant glioblastoma, perianal carcinoma, oral melanoma, thyroid carcinoma, soft tissue carcinoma, cancer ulceration, nasal tumor, or gastrointestinal cancer, for example.

In another embodiment, there is provided herein a composition comprising any of the MSC or MSCs as described herein, and at least one of a pharmaceutically acceptable carrier, diluent, excipient, cell media, or buffer. In another embodiment, there is provided herein a theranostic agent, and/or a kit, comprising any of the MSC or MSCs of any embodiment described herein.

### BRIEF DESCRIPTION OF DRAWINGS

These and other features will become more apparent from the following description in which reference is made to the appended drawings wherein:

FIGS. 1A-C depict generation of CDy::UPRT_AT-MSCs with an embodiment of a LPEI based transfection method. Enhancers enabled high expression of CDy::UPRT in AT-MSC. FIG. 1A shows AT-MSCs in 6 well culture vessels were transfected with 1 μg of CDy::UPRT::GFP pDNA complexed with LPEI (1 μg of DNA to 5 μL of LPEI). After centrifugation, transfection mixture was replaced with fresh media (with or without TrafEn). One day later, representative images were acquired and cells were trypsinized for FACS analysis. Results are presented as mean±SD (n=4). Untransfected AT-MSCs served as negative control. Significant differences between the transfection conditions were calculated using two tailed Student's t-test. **P<0.01. FIG. 1B shows AT-MSCs (LOT00088) cultured in 24-well vessels were transfected at various amount of CDy::UPRT expression plasmid with LPEI or Lipofectamine3000, using centrifugation or manufacturer's protocol respectively. After 24 h incubation, cells were fixed with 4% paraformaldehyde and stained for CDy (green) and nucleus (Hoechst stain, blue). Representative images are shown. Bar represents 400 μm. FIG. 1C: AT-MSCs were transfected with LPEI/CDy::UPRT polyplexes in the presence of TrafEn. One or seven day post modification, the cells were lysed for immunoblotting analysis with antibody targeting CDy. Actin was used as endogenous control for sample loading;

FIGS. 2A-B are bar graphs depicting CDy::UPRT expression rendering modified AT-MSC sensitive to 5FC and 5FU. FIG. 2A shows CDy::UPRT_AT-MSCs were treated with 150 μg/mL of 5FC for the indicated time. The cell viability at each time point was measured by standard MTS assay. At various time points, samples without 5FC treatment served as control. FIG. 2B shows Sensitivity to 5FU was compared between unmodified and modified AT-MSCs after 5 day of culture in the presence of 100 μg/mL 5FU. MTS assay were used to measure cell viability post treatment. Conditions without 5FU treatment was taken as 100%. Results were presented as mean±SD (n=4). Significant differences in cell viability between AT-MSCs and CDy::UPRT_AT-MSCs were calculated using the two tailed student's t-test. **, p<0.005; FIGS. 3A-C show CDy::UPRT expression does not affect standard immunophenotypic profile and differentiation potential. FIG. 3A shows AT-MSCs and CDy::UPRT_AT-MSCs were labelled with fluorophore-conjugated antibodies and analysed by flow cytometry, according to the manufacturer's instructions. Isotype antibodies served as respective controls. Histograms demonstrated the merged profiles of isotypes (Red), unmodified AT-MSCs (Green) and CDy::UPRT_AT-MSCs (Blue). FIG. 3B shows both cell types were cultured in medium supplemented for osteogenic differentiation for 14 days, following manufacturer's recommendations. At the end of incubation, cells were stained with Alizarin red S. Calcium deposits stained with Alizarin red S were one of the phenotype indicating differentiated AT-MSCs. FIG. 3C shows unmodified and CDy::UPRT expressing AT-MSCs were cultured in medium containing components for adipogenic differentiation. Fourteen days later, cells were stained with Oil Red-O. This dye stained for oil droplets visible in the cells, indicative of adipogenic differentiation. The images were captured at 20× magnification;

FIGS. 4A-B show CDy::UPRT expression does not affect migration capability of AT-MSCs. FIG. 4A shows migratory property of MSCs was evaluated using cell invasion assay. Firstly, 200 k or 400 k of target cells were plated in 24 well vessels in DMEM supplemented with 10% FBS. Six hours later, cell cultures were washed once with 1×PBS and replaced with serum free DMEM. CDy::UPRT_AT-MSCs (modified one day before the experiment) and non-modified AT-MSCs were loaded onto matrigel-coated cell inserts. The inserts were transferred to the target cell cultures respectively. Twenty four hours later, cell invasion was evaluated under microscope by taking fluorescent images of cells stained with Hoechst 33342. The number of migratory AT-MSCs was calculated. Graph presents mean of migratory cells per frame (n=3). HEK293T were used as negative control. Significant differences between the 200,000 and 400,000 target cells were calculated using two tailed Student's t-test. **, P<0.01. FIG. 4B shows images of the migrated CDy::UPRT_AT-MSCs stained with Hoechst 33342 were taken at 10× magnification. Scale bar represents 400 μm;

FIGS. 5A-C show selective cytotoxic anticancer effect mediated by CDy::UPRT_AT-MSC/5FC on cancer cells in vitro. FIG. 5A shows CDy::UPRT_AT-MSCs were cocultured with U251-MG, MB-MDA231 or MKN1 in DMEM supplemented with 2% FBS, in the presence or absence of 150 μg/mL 5FC. The therapeutic cells and cancer cell lines were mixed at ratios of 1 CDy::UPRT_AT-MSC to 5, 10, 50, 100 cancer cells. Five days later, proliferation inhibition was evaluated spectrophotometrically by standard MTS assay. The Efficiency of Proliferation Inhibition is defined as 100%−(sample/control×100%). Conditions without 5FC treatment served as controls. Graph bar represents mean (n=4), +SD. FIG. 5B shows bright field of the mixed cultures (1 MSC to 10 cancer cells) taken at the end of experiment. Scale bar represents 400 μm. FIG. 5C shows cytotoxic anticancer effect of CDy::UPRT_AT-MSCs or AT-MSCs on MB-MDA-231 were evaluated by indirect coculture. Equal number of therapeutic cells and MB-MDA-231 were seeded in the transwell and 24 well plates, respectively. Cells were cocultured in DMEM supplemented with 2% FBS and 100 μg/mL 5FC for 4 days. After which, transwells were removed and the remaining cells on the culture plates were stained with Hoechst 3222. The fluorescence readout was captured with microplate reader. Efficiency of Proliferation Inhibition (%) was defined as 100%−(conditions with 5FC/respective conditions without 5FC×100%). Relative fluorescence units collected from 9 areas of biological triplicate were shown as mean±SEM. Graph represents results collected from 9 areas of each well, mean+SEM. Respective images of the remaining cancer cells on 24 well plate are shown. Scale bar represents 400 μm;

FIGS. 6A-C show variable cytotoxic anticancer effect mediated by CDy::UPRT_AT-MSC/5FC generated with different transfection methods. AT-MSCs (250,000 cells) were transfected with 1 μg CpG free CDy::UPRT expression plasmid mediated by LPEI (with or without TrafEn) and Lipofectamine 3000. One day post transfection, CDy::UPRT_AT-MSCs were cocultured with U251-MG, MB-MDA231 or MKN1 in DMEM supplemented with 2% FBS, in the presence or absence of 150 μg/mL 5FC. The therapeutic cells and cancer cell lines were mixed at ratios of 1 CDy::UPRT_AT-MSCs to 1 (FIG. 6A), 5 (FIG. 6B), 10 (FIG. 6C) cancer cells. Five days later, proliferation inhibition was evaluated spectrophotometrically by standard MTS assay. Conditions without 5FC treatment served as controls. The Efficiency of Proliferation Inhibition is defined as 100%−(sample/control×100%). Graph bar represents mean (n=4), ±SD. Significant differences between conditions with LPEI+TrafEn and other methods were calculated using two tailed Student's t-test. **, P<0.01;

FIGS. 7A-C depicts long term expression enables sustainable anticancer efficiency of CDy::UPRT_AT-MSCs. AT-MSCs (250,000 cells) were transfected with 1 μg CpG free CDy::UPRT expression plasmid mediated by LPEI in the presence of TrafEn. One day (FIG. 7A) and seven days (FIG. 7B), modified AT-MSCs were collected and cocultured with MKN1 and MKN28 cell lines at the ratio of 1 MSC to 5 or 10 cancer cells, in the presence or absence of 150 μg/mL 5FC. The proliferation inhibition was evaluated spectrophotometrically by standard MTS assay after 5 days of incubation. Conditions without 5FC treatment served as controls. The Efficiency of Proliferation Inhibition is defined as 100%−(sample/control×100%). Graph bar represents mean (n=4), ±SD. FIG. 7C shows one or seven day post modification, the cells were lysed for immunoblotting analysis with antibody targeting CDy. Actin was used as endogenous control for sample loading. Cell lysates of AT-MSC were collected 1 and 7-day post transfection. The expression of CDy::UPRT was accessed using western blot analysis. In a parallel experiment, modified AT-MSCs were collected on day one (A) or seven (B) days post transfection.

FIGS. 8A-B show TrafEn enabled efficient LPEI based transfection in AT-MSCs. FIG. 8A shows LPEI/pCMV-GFP polyplex or Lipofectamine 3000/pCMV-GFP lipoplex were prepared at various amount of pDNA. AT-MSCs were transfected by LPEI (1 μg pDNA to 10 μL LPEI) or Lipofectamine 3000 following centrifugation protocol or manufacturer's instruction respectively. Fluorescence intensity (RFU) of the GFP expression was measured spectrophotometrically (Ex475/Em509) at nine areas of each biological replicates (n=3). Graph represents mean of RFU+SEM. Reduction in cell number with increasing DNA amount was seen. FIG. 8B shows AT-MSCs were transfected with LPEI complexed with 200 ng pCMV-GFP in the presence or absence of TrafEn. Twenty-four hours later, cells were trypsinized, pelleted and resuspended in 1×PBS for flow cytometry analysis. Transfection efficiency was calculated as the percentage of GFP positive cells normalized to the total number of cells as quantified by FACS. Bar graph represents mean±SD, n=3. Bright field and fluorescent images were captured. Representative images are presented;

FIG. 9 shows high transfection efficiency in AT-MSC isolated from different donor. AT-MSC was isolated from female donor, age 31-45 (LOT00061, Roosterbio). LPEI/pCMV-GFP polyplexes were prepared at various amount of pDNA at the ratio of 1 μg pDNA to 5 μL LPEI. One day (24 hours) later, representative images were acquired, then cells were trypsinized, pelleted and resuspended in 1×PBS for flow cytometry analysis. Transfection efficiency was calculated as the percentage of GFP positive cells normalized to the total number of cells as quantified by FACS. Bar graph represents mean±SD, n=3. Fluorescent images were captured. Representative images are presented;

FIG. 10 shows prolonged expression CDy::UPRT::GFP in AT-MSCs. AT-MSCs were transfected with PEI polyplexes of 1.25 μg of pDNA expressing fused CDy::UPRT::GFP in the presence of TrafEn. On 1, 2, 3, 5, 8 day post transfection, the fluorescent and bright field images were captured. Fluorescence intensity (RFU) of the GFP expression was measured spectrophotometrically (Ex475/Em509) at nine areas of the cell culture. Graph represents mean of RFU+SD for two biological replicates. Significant differences between the GFP expressions on various day post transfection were calculated using two tailed Student's t-test. **P<0.01;

FIG. 11 show adipogenic differentiation of CDy::UPRT::GFP expressing AT-MSC. AT-MSCs were transfected with PEI polyplexes of 1.25 μg of pDNA expressing fused CDy::UPRT::GFP in the presence of TrafEn. Twenty four hour post transfection, the media was replaced with adipogenic differentiation media. Fourteen days later, cells were stained with Oil Red-O. The modified AT-MSCs as indicated with GFP expression display visible oil droplets, suggesting multipotency of AT-MSC remain unchanged post transfection;

FIGS. 12A-C show comparable anticancer efficiency of CDy::UPRT_AT-MSC/5FC and 5FU. The anticancer effect was evaluated in U251-MG (FIG. 12A), MDA-MB-231 (FIG. 12B) and MKN1 (FIG. 12C). The therapeutic efficacy (anticancer effect) of CDy::UPRT_AT-MSCs in combination with 5FC was analysed by coculture of equal number of CDy::UPRT_AT-MSCs and cancer cell lines (2000 U251-MG, 5000 MDA-MB-231 and MKN1). One day later, the culture media was replaced with DMEM supplemented with 2% FBS and various concentrations of 5FC (5, 10, 50, 100 μg/mL). On the other hand, 4000 U251-MG, 10000 MDA-MB-231 and MKN1 were seeded 24 h before 5FU treatment. The cell lines were treated by 5, 10, 50, 100 μg/mL of 5FU in DMEM supplemented with 2% FBS. After 5 days of incubation, the cytotoxic effect were evaluated qualitatively by standard MTS assay. Conditions without treatment of 5FC and 5FU served as negative control that were set as 100%. Graph represents mean±SD, n=4;

FIGS. 13A-B show selective proliferation inhibition of CDy::UPRT_AT-MSC/5FC on cancer cell lines. CDy::UPRT_AT-MSCs were cocultured with HS738T (ATCC, CRL-7869), AGS, MKN28, HS746T, NUGC3 and MKN45 (kindly provided by Dr. Yong Wei Peng, National University Cancer Institute, Singapore). FIG. 13A shows the mixed cultures were incubated DMEM supplemented with 2% FBS, in the presence or absence of 150 μg/mL 5FC. The therapeutic cells and cancer cell lines were mixed at ratios of 1 CDy::UPRT_AT-MSC to 10 cancer cells. Five days later, proliferation inhibition was evaluated spectrophotometrically by standard MTS assay. Conditions without 5FC treatment served as controls. The Efficiency of Proliferation Inhibition is defined as 100%−(sample/control×100%). Graph bar represents mean (n=4), ±SD. FIG. 13B shows bright field images of the mixed cultures taken at the end of experiment. Scale bar represents 400 μm;

FIGS. 14A-B show comparable transfection efficiency and anticancer efficiency in stem cells from different sources. Adipose tissue (AT, Roosterbio), bone marrow (BM, Roosterbio), and UC (Umbilical cord, ATCC) derived MSCs were transfected with the centrifugation protocol in the presence of TrafEn. Twenty four hour post transfection, cells were trypsined and collected for western blot analysis (FIG. 14A). The cells were lysed for immunoblotting analysis with antibody targeting CDy and Actin. FIG. 14B shows in the same experiment, cells were harvested for coculture study with various cancer cell lines at the ratio of 1 MSC to 50 cancer cells. Cells were cocultured in the media containing 100 μg/mL of 5FC for 5 days. At the end of incubation, remaining cell number was evaluated spectrophotometrically by measuring the RFU of cells stained with Hoechst 33342 at wavelength Ex340/Em488. Conditions with unmodified MSCs serve as control. Percentage of proliferation inhibition was calculated according. Graph represents data collected from quadruplicates, mean±SEM;

FIG. 15 depicts comparable transfection efficiency and anticancer efficiency in various stem cells modified to express HSV-TK. AT-, BM- and UC-MSCs were transfected with the centrifugation protocol in the presence of TrafEn. One microgram of pSELECT-zeo-HSV1tk (InvivoGen) was used to transfect 250,000 MSCs. Twenty four hours post transfection, MSCs were harvested for co-culture study with various cancer cell lines at the ratio of 1 MSC to 50 cancer cells. Cells were co-cultured in the media containing 100 μg/mL of prodrug Ganciclovir (InvivoGen) for 5 days. At the end of incubation, remaining cell number was evaluated spectrophotometrically by measuring the RFU of cells stained with Hoechst 33342 at wavelength Ex340/Em488. Conditions with unmodified MSC serves as control. Percentage of proliferation inhibition was calculated according. Graph represents data collected from quadruplicates, mean±SEM;

FIGS. 16A-B show reduction of CDy::UPRT expression overtime with expression vector containing CpG islands. AT-MSC (250,000 cells) were transfected with 1 μg of pSELECT-zeo-FcyFur (InvivoGen) according to the centrifugation protocol, in the presence of TrafEn. One, three and seven days post-transfection, cells were harvested for western blot analysis (FIG. 16A) and co-culture experiment (FIG. 16B). For the co-culture experiment, the CDy::UPRTs modified AT-MSCs were cultured with U-251MG and MDA-MB-231 cells at the ratio of 1 MSC to 1, 5, or 10 cancer cells in the DMEM supplemented with 2% FBS and 100 μg/mL 5FC. Five days later, proliferation inhibition was evaluated spectrophotometrically by standard MTS assay. Conditions without 5FC treatment serve as control. Percentage of proliferation inhibition was calculated according. Graph represents data collected from quadruplicates, mean+SEM;

FIG. 17 shows an illustration of an exemplary embodiment of a protocol for MSC transfection;

FIG. 18 depicts cell viability and transfection efficiency at various DNA amounts. AT-MSC was transfected without centrifugation. The genetic modification efficiency and cell viability was determined with flow cytometry analysis;

FIG. 19 depicts long term of CDy::UPRT in AT-MSC transfected with non-centrifugation protocol. The genetic modification efficiency was determined with flow cytometry analysis;

FIGS. 20A-B depicts compatibility of polymer to different type of MSC: UC-MSC (FIG. 20A) and BM-MSC (FIG. 20B). MSCs were incubated with transfection mixture for 24 h, without centrifugation;

FIG. 21 depicts reducing cellular viability with increasing DNA and polymer amount. AT-MSCs were transfected by various polymers, without centrifugation. The concentration of Linear PEI (<200 kDa) is 1 ug/uL;

FIG. 22 shows reducing cellular viability with increasing DNA and polymer amount. UC-MSCs were transfected by various polymers, without centrifugation. The concentration of Linear PEI (<5 kDa) is 10 ug/uL;

FIGS. 23A-B show high expression level per cell with TrafEn method. U2OS cells (FIG. 23A) or AT-MSC cells (FIG. 23B) were infected with lentivirus and incubated for 5 days (left panel of FIG. 23B). In the same set of experiment, the cells from a separate culture were transfected with PEI in the presence of TrafEn on day 4 (right panel of FIG. 23B). Fluorescent images of the infected and transfected cells were taken on day 5. For AT-MSC, the genetic modification efficiency of lentivirus and TrafEn method was further determined with flow cytometry analysis. Higher number of cells expressed high level of GFP in AT-MSC transfected with the TrafEn method (right panel of FIG. 23B);

FIGS. 24A-B show a graph of number of transfected MSCs obtained in different cell vessel size (FIG. 24A) and a good correlation of number of MSCs & vessel size (FIG. 24B);

FIG. 25 is a schematic depicting development of an integrated process for the production of high numbers of transfected MSC using non-viral transfection method. Factors for consideration and/or optimization to achieve the goal is presented. Due to the variability of cells, a panel of TrafEn compatible polymers (for example, PEI) may be screened to obtain an optimized formulation for high transfection efficiency, low cytotoxicity, prolonged expression and scalable in production;

FIG. 26 depicts cell viability and transfection efficiency at various DNA amounts. The genetic modification efficiency and cell viability was determined with flow cytometry analysis;

FIGS. 27A-B show CDy::UPRT expression does not affect standard immunophenotypic profile and differentiation potential. FIG. 27A shows CDy::UPRT_AT-MSCs were labelled with fluorophore-conjugated antibodies and analysed by flow cytometry. Isotype serves as negative control. FIG. 27B shows both cell types were cultured in medium supplemented for adipogenic differentiation and osteogenic differentiation for 14 and 21 days, respectively. At the end of incubation, cells were stained with Oil Red-O (Adipogenic) or Alizarin red S (Osteogenic). Oil Red-O stained for oil droplets visible in the cells, indicative of adipogenic differentiation. Calcium deposits stained with Alizarin red S were one of the phenotype indicating differentiated AT-MSCs;

FIG. 28 relates to FIG. 8A above. Then, the adherent cells were trypsinized and stained with Propidium Iodide (PI) and Hoechst 33342 (H33342). The cell viability and total adherent cells were determined with NC-3000 cell counter, according the manufacturer's protocol. Un-transfected population serves as control. Cell viability (%) represents percentage of PI negative cells. Percentage of total adherent cells were calculated in relative to control, which was set at 100%. Data are expressed as mean+SD of experiment performed in biological triplicate. Significant differences between control and transfected samples were calculated using the two tailed student's t-test. **, p<0.05;

FIG. 29 relates to the results shown in FIG. 8B above. In the same experiment, total number of adherent cells (left) and cell viability (right) of each condition was determined with NC-3000 cell counter. Un-transfected population serves as control. Results are presented as mean±SD, n=3;

FIG. 30 relates to FIG. 1 above. In the same experiment, total number of the cells and cell viability of each condition was determined with NC-3000 cell counter. The percentage of total adherent cells in transfected population at control (Un-transfected) was calculated. Data represented mean±SD, n=3;

FIG. 31 shows comparable anticancer efficiency of MSC modified with CD::UPRT and CD::UPRT::GFP. MSC (200,000 cells) were transfected with 1 μg of CDy::UPRT or CD::UPRT::GFP, in the presence of Enhancer. One-day post transfection, U-251MG cells were co-cultured with CD::UPRT_MSC at a ratio of 1:1, 5, or 10 (MSC:cancer cells) in DMEM supplemented with 2% FBS, with or without 100 μg/mL 5FC. Five days later, proliferation inhibition in the treatment conditions was evaluated spectrophotometrically by standard MTS assay. Conditions without 5FC treatment serve as control, which was set as 0%. Proliferation inhibition (%) was calculated in relative to control. Data collected from quadruplicates are expressed as mean+SD;

FIG. 32 shows in vivo anti-tumoural effect of CD::UPRT_AT-MSCs in the presence of 5-fluorouracil (5-FU). To establish s.c tumour, 5×10⁶ Temozolomide resistant U-251MG cells were injected subcutaneously in dorsal flank regions. When tumour reached the target size, 1×10⁶ CD::UPRT_AT-MSC or MSC were injected directly to the s.c. tumour. One day later, 500 mg/kg/day of 5FC was administered daily for 4 consecutive day. The size of s.c tumor was measured with digital caliper on day 7, 11, 15 post MSC administration. Prodrug only group serves as control group. Tumor volume (mm³) was calculated according to the standard formula of V=(W(2)×L)/2. (A) The box and whisker bar graph displays the distribution of tumour volume measured from n=5 from each group. Tumor volume in treatment group (CD::UPRT_AT-MSC/5-FU) showed a statistically significant difference (P<0.05) on day 7, 11, 15. (B) At the end of experiment, mice were euthanized. The tumours were extracted and fixed with 4% PFA. Image display tumours (n=5) extracted from each group;

FIG. 33 shows duration of expression and comparison of killing efficiency based on transfection efficiency. AD-MSCs in 24 well culture vessels were transfected with various DNA amount (200 ng-400 ng), using PEI derivative (polymer) with or without the addition of TrafEn. Two days post transfection, cells were trypsinized for FACS analysis. Both (A) % CDUPRTGFP+, and (B) % cell viability, % PI−, were presented. Results are presented as mean±SD (n=3). Non transfected AT-MSC served as negative control. (C) % CD:UPRT:GFP positive cells post harvesting in various transfection conditions, analysis is done using FACS. (D) % Cell viability of co-culture of U251-MG with MSCs transfected with different transfection conditions. Results are presented as mean±SD (n=6);

FIG. 34 shows phenotype of MSCs post-transfection. (A) Expression of CD markers (CD90, CD74, CD105, CD14, CD20, CD34 and CD45) for naïve MSCs (left) and CD:UPRT:GFP MSCs (right), the isotype control was used as a negative control for the FACS analysis. (B) Representative images of Alizarin Red S staining for Osteogenic differentiation (top) and Oil red O staining for adipogenic differentiation (middle) for CD:UPRT:GFP MSCs, overlay of GFP image and Oil Red O staining (bottom) was also shown for the adipogenic differentiation. White arrows point towards CD:UPRT:GFP-expressing cells with Oil Red O staining 14 days post differentiation (C) Fold change of number of migrated naïve and modified AD-MSCs towards U251-MG over fibroblast. The significant difference between the two groups were calculated using unpaired, two-tailed Student's t-test. n.s. represents p-value >0.05 and therefore not significant;

FIG. 35 shows Cytotoxicity of CDUPRTGFP MSC/5-FC against TMZR glioma. Transfected AD-MSCs were cocultured with glioma cell lines (A) U251-MG and U251-MGTMZR40, (B) U87-MG and U87-MGTMZR40, (C) HGCC cell lines and (D) Fibroblasts. Cell viability of co-culture was determined upon seven days incubation with 100 μg/mL 5-FC at different MSC: cancer or fibroblast cell ratio. Results are presented as mean±SD (n=6);

FIG. 36 shows cytotoxicity of CDUPRTGFP MSC/5-FC against U251-MG^{TMZR40} in vivo. (A) Tumour volume was measured before treatment and up to 15 days post treatment (B) Tumour weight was measured upon harvest 15 days post treatment (C) % Change of mice weight was measured before treatment and up to 15 days post treatment. Results are presented as mean±SEM (number of mice is at least 6). The significant differences between tumour volumes and weights from naïve MSCs and different number of CD:UPRT:GFP_MSCs treatment were calculated using unpaired, two-tailed Student's t-test. p-value <0.05 is represented by * while p-value <0.01 is represented by ** and p-value <0.005 is represented by ***. n.s. represents p-value >0.05 and therefore not significant;

FIG. 37 shows application of CD::UPRT::GFP_MSC in conjunction with 5FC as a therapeutic modality for TMZ resistant glioblastoma (U251-MG^{TMZR40}). For a therapeutic regimen, 1×10⁶ therapeutic cells or native cells were injected intratumour ally (Day 0). One day later, the mice received once daily intraperitoneal injections of 500 mg 5FC/kg/day for 4 days. Third day after the last dose of 5FC, the mice were then again injected with the engineered stem cells, and the cycle was repeated for the duration of the experiment. After 3 cycles (50 days after tumour induction or 36 days after first MSC injection) the experiment was terminated. (A) Measurement of tumour volume on indicated days post injection of MSC (B) Image illustrates the tumour size 36 days after the first MSC injection. (C) Weight of mice over the course of experiment;

FIG. 38 shows perianal carcinoma treatment data. Route of administration was intratumoural injection of canine CD::UPRT::GFP_MSC. Latest update (January 2020): alive, recurrence not reported;

FIG. 39 shows oral melanoma treatment data. Route of administration was intratumoural injection of canine CD::UPRT::GFP_MSC. Latest update (January 2020): alive;

FIG. 40 shows thyroid carcinoma treatment data. Route of administration was intratumoural injection of canine CD::UPRT::GFP_MSC. Latest update (June 2019): alive;

FIG. 41 shows soft tissue sarcoma (cancer ulceration) treatment data. Route of administration was intratumoural injection of canine CD::UPRT::GFP_MSC. Latest update (November 2018): alive, no recurrence reported. Ultrasound report on 14-11-2018: Presence of a well-defined hypoechoic round mass on the left anal area measuring 4×3×2 cm. No adhesion to the surrounding or deeper organs. No metastasis found, especially in the sublumbar lymph nodes. Few tiny 1.5 mm uroliths in the bladder, few are in the prostatic urethra. Other organs are normal. Complete Remission to date;

FIG. 42 shows nasal tumour treatment data. Route of administration was intratumoral injection of canine CD::UPRT::GFP_MSC. Latest update (January 2020): alive;

FIG. 43 shows gastrointestinal cancer treatment data. Route of administration was intravenous infusion of canine CD::UPRT::GFP_MSC. Latest update (July 2019): alive. From the ultrasound report despite the fact there is second growth, the original growth has decreased markedly;

FIG. 44 shows MSC types from different commercial sources/collaborations were modified with vector containing GFP transgene. Graph bar displays % of GFP+ population as measured by Flow cytometry. ;

FIG. 45 shows MSC from different sources were modified to express CD::UPRT::GFP;

FIG. 46 shows linearity in scale up of AD-MSCs and UC-MSCs on flat-bed surfaces. (A) Number of transfected live cells were plotted against the surface area of vessel. (B) Representative images of % GFP+from FACS analysis for both AD and UC-MSCs. (C) Percentage of transfection in different culture vessels;

FIG. 47 shows results exploring different microcarriers in AD MSCs. (A) Description of the microcarriers used (B) Number of live cells grown on different microcarrier at different days were plotted;

FIG. 48 shows enhancement of transfection on microcarriers. MSCs were seeded on microcarriers at 1.9 cm\hat{ }2 and transfected with varying DNA amount and addition of enhancers. (A) Transfection efficiency, % GFP+ and % PI− was plotted and (B) representative images were taken at 4× magnification;

FIG. 49 shows different speed affecting microcarrier scale-up. (A) % Transfection efficiency, % GFP+, and cell viability, % PI− are presented. Results are presented as mean±SD (n=3). Non transfected AD-MSC served as negative control. (B) Representative images of transfected cells were taken at 4× magnification;

FIG. 50 shows results of comparison of CD::UPRT::GFP expression and anticancer efficiency of AT-MSC modified by lentivirus or TrafEn mediated transfection method. (A) Three days post infection, MSC were subjected to 1 ug/mL puromycin selection for 2-weeks. After the establishment of MSC stably expressed CD::UPRT::GFP, another set of experiment was set up to generate CD::UPRT::GFP_MSC by TrafEn mediated transfection. Two days post transfection, fluorescent images of modified MSC were captured. (B) After which, both cultures were harvested and subjected to (B) FACS analysis and (C, D) coculture study. The graph bar represents cancer killing efficiencies at various ratios of 1 MSC to 1, 5, 50, 100 cancer cells, obtained through MTS assay. Significant differences in cancer killing efficacies of CD::UPRT::GFP_MSC generated by lentivirus versus TrafEn method, was assessed with two-tailed Student's t-test; **, p-value<0.005; *, p-value<0.05. The bright field images were taken at the end of the coculture experiment;

FIG. 51 shows results of a compassionate use treatment which was performed on a 46 year old patient having recurrent clear cell carcinoma. The subject was treated by intratumoral injection of CD::UPRT::GFP expressing MSCs as described herein;

FIG. 52 shows a schematic depiction of a typical Centrifugation/Spinning-based transfection method (top), as compared with examples of non-centrifugation/spinning transfection methods (bottom). Data collected for cells treating according to such approaches is also provided (see Example 11);

FIG. 53 shows a schematic depiction of a workflow for cryopreserving modified MSCs (prepared using TrafEn) so as to allow for long term storage thereof. As shown, modified MSCs may be placed in cryopreservation storage. When needed, the cells may be removed from storage and prepared for use by thawing in a hypothermic solution; and

FIG. 54 shows results for cell viability (A), expression level (B), and functional activity (C) of modified MSCs that were cryopreserved and then thawed as shown in FIG. 53. As shown, the modified MSCs retained high cell viability and expression level after cryopreservation and preservation in hypothermic solution up to 72 h.

### DETAILED DESCRIPTION

Described herein are methods for transfecting mesenchymal stem cells with a nucleic acid construct from which one or more functional genes are expressed. Also described are transfected mesenchymal stem cells and populations of mesenchymal stem cells, uses thereof, methods for the treatment of diseases or disorders such as cancer using such transfected stem cells, as well as kits and compositions relating thereto. It will be appreciated that embodiments and examples are provided for illustrative purposes intended for those skilled in the art, and are not meant to be limiting in any way.

Stem cells modified to express therapeutic genes, or other genes of interest, are desirable for a number of different therapeutic and non-therapeutic applications. One example is in the field of prodrug gene therapy, aiming to provide modified stem cells expressing an exogenous enzyme capable of converting inactive prodrug to an active therapeutic form at a site where the modified stem cells are introduced into a subject or patient. Traditionally in the field of prodrug gene therapy, virus-based gene modification approaches have been the favoured approach for modifying stem cells such as MSCs in preclinical and clinical studies, since non-viral approaches have generally provided poor transfection efficiency. Indeed, many preclinical studies and clinical trials have exploited viral vectors as gene delivery vehicles for stem cell modification. While viruses may enable sustained expression of transgene, cells infected with virus typically have a low payload of transgene per cell (<10 copies/cell). Higher copy of transcriptional units is often desirable, as this may result in higher transgene expression, which may improve the payload of cell vehicles in delivering therapeutic agents. Production of clinical grade virus may be laborious and often involves generation as well as certification of a master cell bank of stable producer lines, thus incurring high cost in gene-cell therapeutics. A bottleneck in manufacturing of viral carrier has impacted the development and commercialization of cell and gene therapies.

While transient transfection may have advantages in terms of higher payload per cell, avoiding antibiotic selection (and potentially weeks of process work) that may cause cell senescence [40] and/or may reduce tumour tropism [41], as well as safety concerns with viral induced MSC transformation [42], non-viral transfection efficiencies in the field have generally been low. Indeed, although non-viral methods may have advantages over viral vectors for ease of production and/or low cost and safety profiles [43], the lack of wide adoption in the field for non-viral MSC modification may be due to the low efficiency of transfection (0-35%) often observed in the art [44, 45]. For instance, due to the poor performance of certain chemical based transfection methods (<5% efficiency) [46], human adipose tissue derived MSCs (AT-MSCs) have been engineered by retrovirus transduction to express CD::UPRT [47, 48].

Virus-based gene modification in such applications has inherent safety risk, production of clinical grade virus can be laborious, and the number of gene copies which may be introduced per cell through viral methods is generally low (often <10 copies per cell). Furthermore, achieving gene modification of stem cells such as MSCs, either virally or non-virally, without causing undesirable changes to phenotype (i.e. multipotency, immunophenotype, tropism, etc. . . . ) of the resultant cells, and while obtaining high transfection efficiency, is another challenge facing the field.

As described in detail herein, the present inventors have now developed methods for transfecting mesenchymal stem cells with a nucleic acid construct from which one or more functional genes are expressed, which are non-viral and which in certain embodiments may provide high transfection efficiency, high copy number per cell, high cell viability, transient expression for extended duration, and/or a substantially unchanged multipotent phenotype. In certain embodiments, such methods may be scalable and/or suitable for large scale clinical production of modified mesenchymal stem cells. Also described in detail herein are transfected mesenchymal stem cells and populations of mesenchymal stem cells, uses thereof, methods for the treatment of diseases or disorders such as cancer using such transfected mesenchymal stem cells, and kits and compositions relating thereto.

Aspects of the invention may include a variety of embodiments including, but not limited to, the following exemplary embodiments:

Embodiment 1. A mesenchymal stem cell (MSC) transfected with a nucleic acid construct from which one or more functional genes are expressed, the MSC having a phenotype in which any one or more of multipotency (e.g. differentiation potential), immunophenotype, and/or cancer tropism phenotypic characteristic(s) is/are substantially unchanged by the transfection of the nucleic acid construct, and the MSC being free of virus-based transfection vehicle materials.
Embodiment 2. A plurality of mesenchymal stem cells (MSCs), wherein at least about 60% of the MSCs are transfected with a nucleic acid construct from which one or more functional genes are expressed, the transfected MSCs having a phenotype in which any one or more of multipotency (e.g. differentiation potential), immunophenotype, and/or cancer tropism phenotypic characteristic(s) is/are substantially unchanged by the transfection of the nucleic acid construct, and the MSCs being free of virus-based transfection vehicle materials.
Embodiment 3. The plurality of MSCs of Embodiment 2, wherein at least about 65%, at least about 70%, at least about 75%, at least about 80%, at least about 85%, at least about 90%, or at least about 95% of the MSCs are transfected with the nucleic acid construct and express the one or more functional genes.
Embodiment 4. The plurality of MSCs of Embodiments 2 or 3, wherein a cell viability of the plurality of MSCs is at least about 70%, at least about 75%, at least about 80%, or at least about 85%.
Embodiment 5. The MSC or MSCs of any one of Embodiments 1-4, wherein the transfected MSC or MSCs are each transfected with an average of at least about 1000, at least about 2000, at least about 3000, at least about 4000, at least about 5000, at least about 6000, at least about 7000, at least about 8000, at least about 9000, or at least about 10000 copies of the nucleic acid construct.
Embodiment 6. The MSC or MSCs of any one of Embodiments 1-5, wherein the one or more functional genes are transiently expressed in the transfected MSC cell or cells.
Embodiment 7. The MSC or MSCs of any one of Embodiments 1-6, wherein the MSC or MSCs are derived from cord blood, neonatal birth-associated tissue, Wharton's jelly, umbilical cord, cord lining, placenta, or other source of MSC cells.
Embodiment 8. The MSC or MSCs of any one of Embodiments 1-7, wherein the MSC or MSCs are adipose tissue-derived MSC (AT-MSC), bone marrow-derived MSC (BM-MSC), or umbilical cord-derived MSC (UC-MSC).
Embodiment 9. The MSC or MSCs of any one of Embodiments 1-8, wherein the nucleic acid construct comprises a CpG-free expression plasmid or other CpG-free expression construct, a scaffold/matrix attachment region (S/MAR), an episomal vector, or an EBNA-1 containing construct.
Embodiment 10. The MSC or MSCs of any one of Embodiments 1-9, wherein the transfected MSC or MSCs transiently express the one or more functional genes for at least about 7, at least about 8, at least about 9, at least about 10, at least about 11, at least about 12, at least about 13, at least about 14, at least about 15, at least about 16, or at least about 17 days following transfection.
Embodiment 11. The MSC or MSCs of any one of Embodiments 1-10, wherein the transfected MSC or MSCs are transfected with the nucleic acid construct using a cationic polymer, a first agent capable of redirecting endocytosed nucleic acids from intracellular acidic compartments, and a second agent capable of stabilizing a microtubular network of the MSC or MSCs.
Embodiment 12. The MSC or MSCs of Embodiment 11, wherein the cationic polymer comprises linear or branched polyethylenimine (PEI), poly(amidoamine) PAMAM, or another cationic polymer, or any combinations thereof.
Embodiment 13. The MSC or MSCs of Embodiment 12, wherein the cationic polymer comprises linear polyethylenimine (LPEI).
Embodiment 14. The MSC or MSCs of any one of Embodiments 11-13, wherein the first agent comprises 1,2-Dioleoyl-sn-glycero-3-phosphoethanolamine (DOPE)/cholesteryl hemisuccinate (CHEMS) (DOPE/CHEMS), 1,2-dipalmitoyl-sn-glycero-3-phosphocholine (DPPC) or another fusogenic lipid, or any combinations thereof.
Embodiment 15. The MSC or MSCs of any one of Embodiments 11-14, wherein the second agent comprises a histone deactylase inhibitor (HDACi), such as a histone deactylase 6 inhibitor (HDAC6i).
Embodiment 16. The MSC or MSCs of any one of Embodiments 11-15, wherein the second agent comprises SAHA (Vorinostat).
Embodiment 17. The MSC or MSCs of any one of Embodiments 1-16, wherein the one or more functional genes comprise a suicide gene.
Embodiment 18. The MSC or MSCs of any one of Embodiments 1-17, wherein the one or more functional genes comprise Cytosine Deaminase (CDy).
Embodiment 19. The MSC or MSCs of any one of Embodiments 1-18, wherein the one or more functional genes comprise uracil phosphoribosyltransferase (UPRT).
Embodiment 20. The MSC or MSCs of any one of Embodiments 1-19, wherein the one or more functional genes comprise both CDy and UPRT.
Embodiment 21. The MSC or MSCs of Embodiment 20, wherein the CDy and UPRT are expressed as a fused construct.
Embodiment 22. The MSC or MSCs of any one of Embodiments 1-21, wherein the one or more functional genes comprise a fluorescent protein.
Embodiment 23. The MSC or MSCs of Embodiment 22, wherein the fluorescent protein comprises green fluorescent protein (GFP).
Embodiment 24. The MSC or MSCs of Embodiment 20, wherein the one or more functional genes comprise CDy, UPRT, and GFP.
Embodiment 25. The MSC or MSCs of Embodiment 24, wherein the CDy, UPRT, and GFP are expressed as a fused construct.
Embodiment 26. The MSC or MSC of any one of Embodiments 1-25, wherein the one or more functional genes comprise herpes simplex virus-1 thymidine kinase (HSV-TK) or another thymidine kinase.
Embodiment 27. The MSC or MSCs of any one of Embodiments 1-26, wherein the phenotype includes tumor and/or cancer tropism properties of the MSC.
Embodiment 28. The MSC or MSCs of any one of Embodiments 1-27, which is sensitive to treatment with 5-fluorocytosine (5FC) or ganciclovir (GCV).
Embodiment 29. The MSC or MSCs of any one of Embodiments 1-28, which convert: a) 5FC to 5-fluorouridine (5FU), 5-fluorouridine monophosphate (FUMP), or both; b) ganciclovir to ganciclovir monophosphate; or c) a combination of a) and b).
Embodiment 30. The MSC or MSCs of any one of Embodiments 1-29, for use in treating cancer, for example lymphoma, clear cell carcinoma, glioblastoma, temozolomide resistant glioblastoma, perianal carcinoma, oral melanoma, thyroid carcinoma, soft tissue carcinoma, cancer ulceration, nasal tumor, or gastrointestinal cancer.
Embodiment 31. The MSC or MSCs for use according to Embodiment 30, wherein the MSC or MSCs are for use in combination with 5FC, 5FU, GCV, or any combination thereof.
Embodiment 32. The MSC or MSCs of any one of Embodiments 1-31, wherein the phenotype comprises an immunophenotype in which the expression of CD surface markers is substantially unchanged after transfection.
Embodiment 33. The MSC or MSCs of Embodiment 32, wherein the transfected MSC or MSCs are plastic-adherent, express CD105, CD73, and CD90 (>95%), lack expression of CD45, CD34, CD14, and HLA-DR surface molecules (<2%), and are capable of differentiating into osteoblasts, adipocytes, and chondroblasts in vitro, satisfying the immunophenotype criteria defined by the International Society for Cellular Therapy (ISCT).
Embodiment 34. The MSC or MSCs of any one of Embodiments 1-33, wherein the transfected MSC or MSCs are undifferentiated.
Embodiment 35. A method for transfecting mesenchymal stem cells (MSCs) with a nucleic acid construct from which one or more functional genes are expressed, the method comprising:

exposing the MSCs to a transfection mixture comprising the nucleic acid construct which is complexed with a cationic polymer;

exposing the MSCs to a first agent capable of redirecting endocytosed nucleic acids from intracellular acidic compartments and a second agent capable of stabilizing a microtubular network of the MSCs; and

incubating the MSCs;

thereby providing MSCs transfected with the nucleic acid construct.
Embodiment 36. The method of Embodiment 35, wherein the MSCs are not centrifuged during exposure to the transfection mixture, to the first agent and second agent, during incubation, or any combination thereof.
Embodiment 37. The method of Embodiment 35 or 36, wherein the step of incubating the MSCs comprises gentle mixing without centrifugation.
Embodiment 38. The method of any one of Embodiments 35-37, wherein the step of incubating the MSCs comprises incubating the MSCs for at least about 2 hours.
Embodiment 39. The method of Embodiment 38, wherein the step of incubating the MSCs comprises incubating the MSCs for about 2 hours to about 48 hours, or about 3 hours to about 24 hours.
Embodiment 40. The method of Embodiment 39, wherein the step of incubating the MSCs comprises incubating the MSCs for about 4 hours to about 18 hours.
Embodiment 41. The method of any one of Embodiments 35-40, wherein the cationic polymer comprises a cationic polymer which has been identified as having low cytotoxicity against the MSCs.
Embodiment 42. The method of any one of Embodiments 35-41, wherein the step of exposing the MSCs to the transfection mixture comprises complexing the nucleic acid construct with the cationic polymer so as to provide the transfection mixture comprising complexed nucleic acid construct, and adding the transfection mixture to the MSCs.
Embodiment 43. The method of any one of Embodiments 35-42, wherein the step of exposing the MSCs to the transfection mixture comprises adding the transfection mixture to the MSCs and incubating the MSCs with the transfection mixture.
Embodiment 44. The method of any one of Embodiments 35-43, wherein the step of exposing the MSCs to the first and second agents comprises replacing the transfection mixture with cell culture media supplemented with the first agent and second agent.
Embodiment 45. The method of Embodiment 44, wherein the cell culture media comprises complete media.
Embodiment 46. The method of any one of Embodiments 35-45, wherein the MSCs are at about 60% confluency, and the MSCs are seeded for about 24 hours prior to exposure to the transfection mixture.
Embodiment 47. The method of any one of Embodiments 35-46, wherein the cationic polymer has a size of about 5 kDa to about 200 kDa.
Embodiment 48. The method of any one of Embodiments 35-47, wherein the cationic polymer comprises linear or branched polyethylenimine (PEI), poly(amidoamine) PAMAM, or another cationic polymer, or any combinations thereof.
Embodiment 49. The method of any one of Embodiments 35-48, wherein the cationic polymer comprises linear polyethylenimine (LPEI).
Embodiment 50. The method of any one of Embodiments 35-49, wherein the first agent comprises 1,2-Dioleoyl-sn-glycero-3-phosphoethanolamine (DOPE)/cholesteryl hemisuccinate (CHEMS) (DOPE/CHEMS), 1,2-dipalmitoyl-sn-glycero-3-phosphocholine (DPPC) or another fusogenic lipid, or any combinations thereof.
Embodiment 51. The method of any one of Embodiments 35-50, wherein the second agent comprises a histone deacetylase inhibitor (HDACi), such as a histone deactylase 6 inhibitor (HDAC6i).
Embodiment 52. The method of Embodiment 51, wherein the second agent comprises SAHA (Vorinostat).
Embodiment 53. The method of any one of Embodiments 35-52, wherein the transfection mixture comprises the complexed nucleic acid construct in serum free DMEM, or in fresh culture media.
Embodiment 54. The method of any one of Embodiments 35-53, wherein the step of exposing the MSCs to the transfection mixture comprises removing a culture media from the MSCs and replacing the culture media with the transfection mixture.
Embodiment 55. The method of Embodiment 35, wherein the step of exposing the MSC to the transfection mixture comprises incubating the MSCs with the transfection mixture under mild centrifugation.
Embodiment 56. The method of Embodiment 55, wherein the mild centrifugation comprises about 200 g for about 5 minutes.
Embodiment 57. The method of any one of Embodiments 35-56, wherein the amount of nucleic acid construct in the transfection mixture to which the MSCs are exposed is between about 200 to about 500 ng per 1.9 cm² surface area.
Embodiment 58. The method of Embodiment 57, wherein the amount of nucleic acid construct in the transfection mixture to which the MSCs are exposed is between about 250 to about 400 ng per 1.9 cm² surface area.
Embodiment 59. The method of Embodiment 58, wherein the amount of nucleic acid construct in the transfection mixture to which the MSCs are exposed is between about 300 to about 350 ng per 1.9 cm² surface area.
Embodiment 60. The method of any one of Embodiments 35-59, wherein a ratio of cationic polymer to nucleic acid construct is about 1 μg to about 30 μg cationic polymer per 1 μg of nucleic acid construct in the transfection mixture.
Embodiment 61. The method of any one of Embodiments 35-60, wherein the one or more functional genes comprise a suicide gene.
Embodiment 62. The method of any one of Embodiments 35-61, wherein the one or more functional genes comprise Cytosine Deaminase (CDy) and/or thymidine kinase (TK).
Embodiment 63. The method of any one of Embodiments 35-63, wherein the one or more functional genes comprise uracil phosphoribosyltransferase (UPRT).
Embodiment 64. The method of any one of Embodiments 35-64, wherein the one or more functional genes comprise both CDy and UPRT.
Embodiment 65. The method of Embodiment 64, wherein the CDy and UPRT are expressed as a fused construct.
Embodiment 66. The method of any one of Embodiments 35-66, wherein the one or more functional genes comprise a fluorescent protein.
Embodiment 67. The method of Embodiment 66, wherein the fluorescent protein comprises green fluorescent protein (GFP).
Embodiment 68. The method of Embodiment 64, wherein the one or more functional genes comprise CDy, UPRT, and GFP.
Embodiment 69. The method of Embodiments 68, wherein the CDy, UPRT, and GFP are expressed as a fused construct.
Embodiment 70. The method of any one of Embodiments 35-69, wherein the one or more functional genes comprise herpes simplex virus-1 thymidine kinase (HSV-TK).
Embodiment 71. The method of any one of Embodiments 35-70, wherein the one or more functional genes are transiently expressed in the transfected MSCs.
Embodiment 72. The method of any one of Embodiments 35-71, wherein the transfected MSCs are each transfected with an average of at least about 1000, at least about 2000, at least about 3000, at least about 4000, at least about 5000, at least about 6000, at least about 7000, at least about 8000, at least about 9000, or at least about 10000 copies of the nucleic acid construct
Embodiment 73. The method of any one of Embodiments 35-72, wherein a phenotype of the transfected MSCs, such as a phenotype comprising any one or more of multipotency, immunophenotype, and/or cancer tropism phenotypic characteristic(s), is/or substantially unchanged by the transfection.
Embodiment 74. The method of Embodiment 73, wherein the phenotype comprises tumor and/or cancer tropism properties of the MSC.
Embodiment 75. The method of Embodiment 73 or 74, wherein the phenotype comprises an immunophenotype in which the expression of CD surface markers is substantially unchanged after transfection.
Embodiment 76. The method of Embodiment 75, wherein the transfected MSCs are plastic-adherent, express CD105, CD73, and CD90 (>95%), lack expression of CD45, CD34, CD14, and HLA-DR surface molecules (<2%), and are capable of differentiating into osteoblasts, adipocytes, and chondroblasts in vitro, satisfying the immunophenotype criteria defined by the International Society for Cellular Therapy (ISCT).
Embodiment 77. The method of any one of Embodiments 35-76, wherein at least about 60%, at least about 65%, at least about 70%, at least about 75%, at least about 80%, at least about 85%, at least about 90%, or at least about 95% of the MSCs are transfected with the nucleic acid construct and express the one or more functional genes.
Embodiment 78. The method of any one of Embodiments 35-77, wherein a cell viability of the transfected MSCs is at least about 70%, at least about 75%, at least about 80%, or at least about 85%.
Embodiment 79. The method of any one of Embodiments 35-78, wherein the transfected MSCs are undifferentiated.
Embodiment 80. The method of any one of Embodiments 35-79, wherein the method is free of virus-based transfection vehicle materials.
Embodiment 81. The method of any one of Embodiments 35-80, wherein the MSCs are derived from cord blood, neonatal birth-associated tissue, Wharton's jelly, umbilical cord, cord lining, placenta, or other source of MSC cells.
Embodiment 82. The method of any one of Embodiments 35-81, wherein the MSCs are adipose tissue-derived MSC (AT-MSC), bone marrow-derived MSC (BM-MSC), or umbilical cord-derived MSC (UC-MSC).
Embodiment 83. The method of any one of Embodiments 35-82, wherein the nucleic acid construct comprises a CpG-free expression plasmid or other CpG-free expression construct, a scaffold/matrix attachment region (S/MAR), an episomal vector, or an EBNA-1 containing construct.
Embodiment 84. The method of any one of Embodiments 35-83, wherein the MSCs transiently express the one or more functional genes for at least about 7, at least about 8, at least about 9, at least about 10, at least about 11, at least about 12, at least about 13, at least about 14, at least about 15, at least about 16, or at least about 17 days following transfection.
Embodiment 85. The method of any one of Embodiments 35-84, wherein the resultant MSCs are sensitive to treatment with 5-fluorocytosine (5FC) or ganciclovir (GCV) or both.
Embodiment 86. The method of any one of Embodiments 35-84, wherein the resultant MSC converts: a) 5FC to 5-fluorouracil (5FU), 5-fluorouridine monophosphate (FUMP) or both; b) ganciclovir to ganciclovir monophosphate; or c) a combination of a) and b).
Embodiment 87. The method of any one of Embodiments 35-86, wherein the one or more functional genes comprise a fluorescent protein, and the method further comprises a step of isolating, selecting, or purifying the transfected MSCs using cell sorting or FACS.
Embodiment 88. The method of any one of Embodiments 35-87, wherein the transfected MSCs are MSCs as defined in any one of Embodiments 1-34.
Embodiment 89. An MSC, or plurality of MSCs, produced by the method of any one of Embodiments 35-88.
Embodiment 90. Use of the MSC or MSCs as defined in any one of Embodiments 1-34 or 89, for treating cancer, for example lymphoma, clear cell carcinoma, glioblastoma, temozolomide resistant glioblastoma, perianal carcinoma, oral melanoma, thyroid carcinoma, soft tissue carcinoma, cancer ulceration, nasal tumor, or gastrointestinal cancer, in a subject in need thereof.
Embodiment 91. The use of Embodiment 90, wherein the MSC or MSCs are for use in combination with 5FC, 5FU, GCV, or any combination thereof.
Embodiment 92. Use of the MSC or MSCs as defined in any one of Embodiments 1-34 or 89, in the manufacture of a medicament for the treatment of cancer, for example lymphoma, clear cell carcinoma, glioblastoma, temozolomide resistant glioblastoma, perianal carcinoma, oral melanoma, thyroid carcinoma, soft tissue carcinoma, cancer ulceration, nasal tumor, or gastrointestinal cancer.
Embodiment 93. The use of Embodiment 92, wherein the MSC or MSCs are for use in combination with 5FC, 5FU, GCV, or any combination thereof.
Embodiment 94. A method for treating cancer, for example lymphoma, clear cell carcinoma, glioblastoma, temozolomide resistant glioblastoma, perianal carcinoma, oral melanoma, thyroid carcinoma, soft tissue carcinoma, cancer ulceration, nasal tumor, or gastrointestinal cancer, in a subject in need thereof, said method comprising:

administering an MSC or MSCs as defined in any one of Embodiments 1-34 or 89 to a region in proximity with a cancer cell of the subject,

wherein the one or more functional genes in the MSC or MSCs contribute to an anticancer effect on the cancer cell.
Embodiment 95. The method of Embodiment 94, wherein the MSC or MSCs are administered simultaneously, sequentially, or in combination with 5FC, 5FU, GCV, or any combination thereof.
Embodiment 96. The method of Embodiment 94 or 95, wherein the one or more functional genes comprise Cytosine Deaminase (CDy), thymidine kinase (TK), or both.
Embodiment 97. The method of any one of Embodiments 94-96, wherein the one or more functional genes comprise uracil phosphoribosyltransferase (UPRT).
Embodiment 98. The method of any one of Embodiments 94-97, wherein the one or more functional genes comprise both CDy and UPRT.
Embodiment 99. The method of Embodiment 98, wherein the CDy and UPRT are expressed in the MSC or MSCs as a fused construct.
Embodiment 100. The method of any one of Embodiments 94-99, wherein the MSC or MSCs transiently express the one or more functional genes for at least about 7, at least about 8, at least about 9, at least about 10, at least about 11, at least about 12, at least about 13, at least about 14, at least about 15, at least about 16, or at least about 17 days following transfection.
Embodiment 101. The method of any one of Embodiments 94-100, further comprising a step of administering 5FC, 5FU, ganciclovir, or any combination thereof, to the subject such that the MSC or MSCs are exposed to the 5FC, 5FU, ganciclovir or combination thereof.
Embodiment 102. The method of any one of Embodiments 94-101, further comprising a step of producing the MSC or MSCs according to a method as defined in any one of Embodiments 35-88 prior to the step of administering the MSC or MSCs.
Embodiment 103. A composition comprising the MSC or MSCs of any one of Embodiments 1-34 or 89, and at least one of a pharmaceutically acceptable carrier, diluent, excipient, cell media, or buffer.
Embodiment 104. A theranostic agent comprising the MSC or MSCs of any one of Embodiments 1-34 or 89.
Embodiment 105. A kit for transfecting a mesenchymal stem cell (MSC) with a nucleic acid construct from which one or more functional genes are transiently expressed, the kit comprising one or more of:

an MSC;

a nucleic acid construct designed for transient expression of one or more functional genes;

a cell culture media;

a cationic polymer;

a first agent capable of redirecting endocytosed nucleic acids from intracellular acidic compartments;

a second agent capable of stabilizing a microtubular network of the MSC;

instructions for performing a method as defined in any one of Embodiments 35-88;

5FC;

GCV; and/or

5FU.

Embodiment 106. The kit of Embodiment 105, wherein the MSC is derived from cord blood, neonatal birth-associated tissue, Wharton's jelly, umbilical cord, cord lining, placenta, or other source of MSC cells.
Embodiment 107. The kit of Embodiment 105 or 106, wherein the MSC is an adipose tissue-derived MSC (AT-MSC), bone marrow-derived MSC (BM-MSC), or umbilical cord-derived MSC (UC-MSC).
Embodiment 108. The kit according to Embodiment 105 or 106, wherein the nucleic acid construct comprises a CpG-free expression plasmid or other CpG-free expression construct, a scaffold/matrix attachment region (S/MAR), an episomal vector, or an EBNA-1 containing construct.
Embodiment 109. The kit of any one of Embodiments 105-108, wherein the cationic polymer comprises linear or branched polyethylenimine (PEI), poly(amidoamine) PAMAM, or another cationic polymer, or any combinations thereof.
Embodiment 110. The kit of any one of Embodiments 105-180, wherein the cationic polymer comprises linear polyethylenimine (LPEI).
Embodiment 111. The kit of any one of Embodiments 105-110, wherein the first agent comprises one or more of DOPC, DPPC, or another fusogenic lipid.
Embodiment 112. The kit of any one of Embodiments 105-111, wherein the first agent comprises 1,2-Dioleoyl-sn-glycero-3-phosphoethanolamine (DOPE)/cholesteryl hemisuccinate (CHEMS) (DOPE/CHEMS), 1,2-dipalmitoyl-sn-glycero-3-phosphocholine (DPPC) or another fusogenic lipid, or any combinations thereof.
Embodiment 113. The kit of any one of Embodiments 105-112, wherein the second agent comprises a histone deactylase inhibitor (HDACi), such as a histone deactylase 6 inhibitor (HDAC6i).
Embodiment 114. The kit of Embodiment 113, wherein the second agent comprises SAHA

(Vorinostat).

Embodiment 115. The kit of any one of Embodiments 105-114, wherein the one or more functional genes comprise a suicide gene.
Embodiment 116. The kit of any one of Embodiments 105-115, wherein the one or more functional genes comprise Cytosine Deaminase (CDy) or thymidine kinase (TK).
Embodiment 117. The kit of any one of Embodiments 105-116, wherein the one or more functional genes comprise uracil phosphoribosyltransferase (UPRT).
Embodiment 118. The kit of any one of Embodiments 105-116, wherein the one or more functional genes comprise both CDy and UPRT.
Embodiment 119. The kit of Embodiment 118, wherein the CDy and UPRT are expressed as a fused construct.
Embodiment 120. The kit of any one of Embodiments 105-119, wherein the one or more functional genes comprise a fluorescent protein.
Embodiment 121. The kit of Embodiment 120, wherein the fluorescent protein comprises green fluorescent protein (GFP).
Embodiment 122. The kit of Embodiment 118, wherein the one or more functional genes comprise CDy, UPRT, and GFP.
Embodiment 123. The kit of Embodiment 122, wherein the CDy, UPRT, and GFP are expressed as a fused construct.
Embodiment 124. The kit of any one of Embodiments 105-123, wherein the one or more functional genes comprise herpes simplex virus-1 thymidine kinase (HSV-TK).
Embodiment 125. The kit of any one of Embodiments 105-124, wherein the cationic polymer comprises a cationic polymer which has been identified as having low cytotoxicity against the MSCs.
Embodiment 126. The kit of any one of Embodiments 105-125, wherein the cationic polymer has a size of about 5 kDa to about 200 kDa.
Embodiment 127. The kit of any one of Embodiments 105-126, wherein a ratio of cationic polymer to nucleic acid construct in the kit is about 1 μg to about 30 μg cationic polymer per 1 μg of nucleic acid construct.
Embodiment 128. The kit of any one of Embodiments 105-124, wherein the kit is for preparing an MSC-based anti-cancer agent.
Embodiment 129. The kit of Embodiment 128, wherein the kit further comprises instructions and/or apparatus for performing a method as defined in any one of Embodiments 94-102.
Embodiment 130. The method according to any one of Embodiments 35-43, 46-53, or 57-88, wherein the method comprises a step of culturing the MSCs in a growth medium, such as a fresh growth medium, before the step of exposing the MSCs to the transfection mixture.
Embodiment 131. The method of Embodiment 130, wherein the step of exposing the MSCs to the transfection mixture comprises adding the transfection mixture to the MSCs without removing the growth medium from the MSCs, and centrifugation is not performed during the steps of exposing and incubating.
Embodiment 132. The method of Embodiment 130 or 131, wherein the step of exposing the MSCs to the first agent and the second agent comprises adding the first and second agent to the MSCs simultaneously, sequentially, or in combination with the transfection mixture.
Embodiment 133. The method of Embodiment 132, wherein the first and second agent are added to the MSCs simultaneously with addition of the transfection mixture to the MSCs, or wherein the first and second agent are mixed with the transfection mixture and added to the MSCs.
Embodiment 134. The method of Embodiment 132, wherein the first and second agent are added to the MSCs shortly after the transfection mixture is added to the MSCs.
Embodiment 135. The method of any one of Embodiments 132-134, wherein the transfection mixture is not removed before the first and second agents are added to the MSCs.
Embodiment 136. The method of any one of Embodiments 130-135, wherein a duration of exposure of the MSCs to the transfection mixture overlaps with a duration of exposure of the MSCs to the first and second agents.
Embodiment 137. The method of Embodiment 136, wherein the transfection mixture is not removed before the first and second agents are added to the MSCs.
Embodiment 138. A method for transfecting mesenchymal stem cells (MSCs) with a nucleic acid construct from which one or more functional genes are expressed, the method comprising:

culturing the MSCs in a growth medium;

adding a transfection mixture comprising the nucleic acid construct which is complexed with a cationic polymer to the MSCs without removing the growth medium from the MSCs;

adding a first agent capable of redirecting endocytosed nucleic acids from intracellular acidic compartments and a second agent capable of stabilizing a microtubular network of the MSCs to the MSCs; and

incubating the MSCs while in contact with all of the transfection mixture, the first agent, and the second agent for an incubation period;

wherein the first and second agents are added to the MSCs simultaneously with the addition of the transfection mixture, sequentially with the addition of the transfection mixture, or in combination with the transfection mixture; and
wherein the MSCs are not centrifuged between the adding of the transfection mixture and expiry of the incubation period;
thereby providing MSCs transfected with the nucleic acid construct.
Embodiment 139. The method of Embodiment 138, wherein the incubation period is at least about 2 hours.
Embodiment 140. The method of Embodiment 138, wherein the incubation period is about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, or about 36 hours, or more.
Embodiment 141. An MSC cell, or plurality of MSC cells, produced by the method of any one of Embodiments 130-140.
Embodiment 142. Use of the MSC or MSCs as defined in Embodiment 141 for treating cancer, for example lymphoma, clear cell carcinoma, glioblastoma, temozolomide resistant glioblastoma, perianal carcinoma, oral melanoma, thyroid carcinoma, soft tissue carcinoma, cancer ulceration, nasal tumor, or gastrointestinal cancer, in a subject in need thereof.
Embodiment 143. Use of the MSC or MSCs as defined in Embodiment 141 in the manufacture of a medicament for the treatment of cancer, for example lymphoma, clear cell carcinoma, glioblastoma, temozolomide resistant glioblastoma, perianal carcinoma, oral melanoma, thyroid carcinoma, soft tissue carcinoma, cancer ulceration, nasal tumor, or gastrointestinal cancer.
Embodiment 144. A method for treating cancer, for example lymphoma, clear cell carcinoma, glioblastoma, temozolomide resistant glioblastoma, perianal carcinoma, oral melanoma, thyroid carcinoma, soft tissue carcinoma, cancer ulceration, nasal tumor, or gastrointestinal cancer, in a subject in need thereof, said method comprising:

administering an MSC or MSCs as defined in Embodiments 141 to a region in proximity with a cancer cell of the subject,

wherein the one or more functional genes in the MSC or MSCs contribute to an anticancer effect on the cancer cell.
Embodiment 145. A composition comprising the MSC or MSCs of Embodiment 141, and at least one of a pharmaceutically acceptable carrier, diluent, excipient, cell media, or buffer.
Embodiment 146. A theranostic agent comprising the MSC or MSCs of Embodiment 141.
Embodiment 147. A kit for transfecting a mesenchymal stem cell (MSC) with a nucleic acid construct from which one or more functional genes are transiently expressed, the kit comprising one or more of:

an MSC;

a nucleic acid construct designed for transient expression of one or more functional genes;

a cell culture media;

a cationic polymer;

a first agent capable of redirecting endocytosed nucleic acids from intracellular acidic compartments;

a second agent capable of stabilizing a microtubular network of the MSC;

instructions for performing a method as defined in any one of Embodiments 130-140;

5FC;

GCV; and/or

5FU.

Transfected Mesenchymal Stem Cells, and Methods and Kits for the Production Thereof

In an embodiment, there is provided herein a mesenchymal stem cell (MSC) transfected with a nucleic acid construct from which one or more functional genes are expressed, the MSC having a multipotent phenotype which is substantially unchanged by the transfection of the nucleic acid construct, and the MSC being free of virus-based transfection vehicle materials.

In another embodiment, there is provided herein a plurality of mesenchymal stem cells (MSCs), wherein at least about 60% of the MSCs are transfected with a nucleic acid construct from which one or more functional genes are expressed, the transfected MSCs having a multipotent phenotype which is substantially unchanged by the transfection of the nucleic acid construct, and the MSCs being free of virus-based transfection vehicle materials.

As will be understood, MSCs may include any suitable MSCs, such as those derived from cord blood, neonatal birth-associated tissue, Wharton's jelly, umbilical cord, cord lining, placenta, or other source of MSC cells. Sources of MSCs may include human, canine, feline, equine and others. In certain embodiments, the MSCs may be one or more of adipose tissue-derived MSCs (AT-MSC), bone marrow-derived MSCs (BM-MSC), or umbilical cord-derived MSCs (UC-MSC), for example.

In certain embodiments, where the modified (transfected) MSCs expressing the functional gene(s) are to be used for treating a subject, it is contemplated that the MSCs that are transfected may comprise MSCs originally derived from the subject to be treated, or from a different source or subject. In certain embodiments, MSCs may be selected for compatibility with the subject to be treated (to avoid, for example, an allergic reaction), and the MSCs may or may not be originally derived from the subject to be treated. In certain embodiments, autologous or allogenic MSCs may be used.

In certain embodiments, the MSCs may be transfected with the nucleic acid construct. As will be understood, in certain embodiments the MSCs may be transiently transfected with the nucleic acid construct (i.e., the nucleic acid construct may be introduced to a location of the cell where the one or more functional genes which it encodes may be expressed in the cell, but the nucleic acid construct is not integrated into the cell genome), or may be stably transfected with the nucleic acid construct (i.e. the nucleic acid construct may be integrated into the cell genome, where the one or more functional genes which it encodes may be expressed in the cell; with or without performing a selection step (for example, antibiotic resistance where such a gene is included with the nucleic acid construct).

In certain embodiments, the MSCs may be transfected using reagents and/or methods as described in detail hereinbelow.

As will be understood, the nucleic acid construct may comprise any suitable nucleic acid sequence or sequences suitable for the particular application, and suitable for encoding the one or more functional genes of interest. In certain embodiments, the nucleic acid construct may comprise generally any suitable plasmid, expression vector, or other expressible nucleic acid sequence which can result in the production of the one or more functional genes/polypeptides which the nucleic acid construct encodes following introduction into a cell. In embodiments where prolonged expression is desirable, nucleic acid constructs designed for long term expression and/or prevention of cellular silencing may be used.

In certain embodiments, the nucleic acid construct may be designed such that the coding region(s) (i.e. the region(s) encoding the one or more functional genes of interest) use codons which are optimized for expression in a particular organism of interest (for example, codons may be optimized for expression in human cells when using human MSCs). In certain embodiments, the nucleic acid construct may be an expressible nucleic acid (i.e. the nucleic acid construct may be designed to result in expression of a polypeptide when introduced or present in a given cell). In certain embodiments, the nucleic acid construct may be DNA or RNA. In certain embodiments, the nucleic acid construct may be a plasmid, expression vector, mRNA (which may, in certain embodiments, include sequence appropriate for translation in a cell of interest such as a start codon, poly-A tail, RBS sequence, and/or others), minicircle DNA, fragments of single stranded or double stranded DNA, or others, with appropriate upstream and/or downstream sequence such that translation, or transcription and translation, of the nucleic acid construct may occur once the nucleic acid construct is introduced to a cell so as to provide the polypeptide(s) of the one or more functional genes.

Suitable expression vector techniques for overexpressing or introducing a particular functional gene/polypeptide into a cell are known in the art (see, for example, Molecular Cloning: A Laboratory Manual (4th Ed.), 2012, Cold Spring Harbor Laboratory Press). As will be known to one of skill in the art, nucleotide sequences for expressing a particular polypeptide may encode or include features as described in “Genes VII”, Lewin, B. Oxford University Press (2000) or “Molecular Cloning: A Laboratory Manual”, Sambrook et al., Cold Spring Harbor Laboratory, 3rd edition (2001). A nucleotide sequence encoding a particular functional gene/polypeptide of interest may be incorporated into a suitable vector, such as a commercially available vector. Vectors may also be individually constructed or modified using standard molecular biology techniques, as outlined, for example, in Sambrook et al. (Cold Spring Harbor Laboratory, 3rd edition (2001)). The person of skill in the art will recognize that a vector may include nucleotide sequences encoding desired elements that may be operably linked to a nucleotide sequence encoding a functional gene/polypeptide. Such nucleotide sequences encoding desired elements may include transcriptional promoters (for example, a constitutive or inducible promoter), transcriptional enhancers, transcriptional terminators, and/or an origin of replication. Selection of a suitable vector may depend upon several factors, including, without limitation, the size of the nucleic acid to be incorporated into the vector, the type of transcriptional and translational control elements desired, the level of expression desired, copy number desired, whether chromosomal integration is desired, the type of selection process that is desired (if any), or the host cell or the host range that is intended to be transformed.

As will be understood, the nucleic acid construct may encode one or more functional genes. The one or more functional genes may comprise generally any suitable functional gene, encoding one or more functional RNA, peptide, polypeptide, or protein of interest. As will be understood, the one or more functional genes will typically be selected to suit the particular application for which the modified mesenchymal stem cells are to be applied. By way of example, where the modified MSCs are to be used in a prodrug gene therapy approach, the one or more functional genes may comprise an enzyme which is capable of converting an inactive or poorly active prodrug into an active form, such that upon exposure of the modified MSC to the prodrug, active drug will form and be able to treat surrounding cells and/or tissues. In certain embodiments, the one or more functional genes may include a suicide gene, which may convert a prodrug to an active form that harms both the modified MSC, and surrounding diseased cells, for example. In another embodiment, the one or more functional genes may comprise one or more cancer therapy genes, or one or more functional genes which are not related to cancer therapy and may have other therapeutic or non-therapeutic functions, for example.

Many examples of prodrug gene therapy systems, including both suitable prodrugs and corresponding functional genes/suicide genes, will be known to the person of skill in the art having regard to the teachings herein. Some examples of functional genes which may be used, and their corresponding prodrugs (where used), are set out in Table 1 as follows:

TABLE 1
Various Examples of Functional Gene/Suicide Gene and Prodrug Systems (adapted from J. Clin.
Invest., 2000, 105(9): 1161-1167, which is herein incorporated by reference in its entirety).
Quantitative data on GDEPT systems

| | | Potency, IC₅₀ | | | | Potential of | Degree of | |
|---|---|---|---|---|---|---|---|---|
| | Enzyme/prodrug | (μM) | | K_{M} (S_{0.5}) | Vₘₐₜ | activation [symbol] | activation [symbol] | Clinical |
| No. | system | Prodrug | Drug | (μM) | (nM/mg/min) | (fold) | (fold) | trial |
| 1 | CA/CPT-11 | 1.6-8.1 | SN-38: | 23-52.9 | 1.43 [symbol] | 150-3,000 | 7-17 | 1 |
| | | | 0.003-0.011 | | | | | |
| 2 | CD/5-FC | 26,000 | 5-FU: 4-23.5 | 17,900 [symbol] | 11.7 [symbol] | 100-8,000 | 70-1,000 | 2 |
| | | | | 800 [symbol] | 68 [symbol] | | | |
| 3 | CPG2/CMDA, | CMDA: | CMBA: 8-65; | CMDA: | CMDA: 583 [symbol] | CMDA: 26- | CMDA: 10- | None |
| | CJS278 [symbol] | 1,700-3,125; | | 3.4 | | 390; | 115; | |
| | | CJS278: | Doxorubicin: | | Doxorubicin: | Doxorubicin: | | |
| | | 0.256 | 0.012 | | 21 | 11 | | |
| 4 | Cyt-450/CP, IF, | CP, | [symbol] | CP: 300; | CP: 39.1; | [symbol] | 5-60 | 1 |
| | ipomeanol, 2-AA | IF ~4,000 | | IF: 480 | IF: 17.8 | | 50-100 [symbol] | |
| 5 | dCK/ara-C | 0.3-0.6 | [symbol] | 25.6 | [symbol] | [symbol] | 2-100 | None |
| 6 | HSV-TK/GCV, ACV | GCV: | GCVTP [symbol] | GCV: 11-15.8 | GCV: 1.3-2.2 [symbol] | [symbol] | 20-1,000 | >21 |
| | | 200-600 | | ACV: 305-375 | ACV: 0.3-0.4 [symbol] | | | |
| 7 | NR/CB1954 | >1,000 | 0.02 [symbol] | 900 | 6.0 [symbol] | >50,000 | 14-10,000 | None |
| 8 | PNP/6-MePdR | >200 | 3.7 | 14-23^{O} | 422-638^{O} [symbol] | 25-1,000 | 40 | None |
| 9 | TP/5′-DFUR | 17 | 5-FUdR; | 325-433 | 0.17-2.28 | 7000 | 165 | [symbol] |
| | | | 0.0023 | | | | | |
| 10 | VZV-TK/ara-M | >2,000 | Ara-MTP | 56 | 680 [symbol] | >2,000 | 55-600 | None |
| | | | <1 [symbol] | | | | | |
| 11 | XGPRT/6-TX, | 6-TX > 50; | [symbol] | [symbol] | [symbol] | [symbol] | 6-TX: >20; | None |
| | 5-TG | 6-TG = 0.5 | | | | | 6-TG: 10 | |

[symbol]

[symbol]

indicates data missing or illegible when filed

Further examples of functional genes which may be used may include any suitable functional gene producing a nucleic acid or polypeptide product which may be useful in treating a disease or disorder of interest. As will be understood, a wide variety of nucleic acids, peptides, polypeptides, and proteins having therapeutic activity will be known to the person of skill in the art and may be included in the nucleic acid constructs as described herein. By way of example, genes that have been introduced into MSCs for cancer therapy in the field, and which may be incorporated into the constructs and methods described herein, may include the following:

TABLE 2
Stem Cell and Suicide Gene Therapy Approaches Relating to
Modification of MSCs in Cancer Therapy, and Corresponding References

| | | | | Gene delivery | Stable |
|---|---|---|---|---|---|
| References | Year | Stem cell type | Gene | method | cell line |
| STEM CELLS; 27: 2320-2330 | 2009 | Bone Marrow MSC | TRAIL | Lentivirus | Y |
| Cancer Res; 69: (10) | 2009 | Bone Marrow MSC | TRAIL | Lentivirus | Y |
| Cytotherapy; 17: 885e896 | 2015 | Bone Marrow MSC | TRAIL | Lentivirus | Y |
| J Gene Med; 10: 1071-1082. | 2008 | Adipose Derived MSC | CDy::UPRT | Retrovirus | Y |
| J of Experimental & Clinical | 2015 | Adipose Derived MSC | CDy::UPRT, | Retrovirus | Y |
| Cancer Research 34: 33 | | | HSVTK | | |
| Cancer Res; 67(13): 6304-13 | 2007 | Adipose Derived MSC | CDy::UPRT | Retrovirus | Y |
| Molecular Therapy 18 1, 223-231. | 2010 | Adipose Derived MSC | CDy::UPRT | Retrovirus | Y |
| European Journal of | 2014 | Bone Marrow & | CDy::UPRT | Retrovirus | Y |
| Cancer 50, 2478-2488 | | Adipose Derived | | | |
| CANCER RESEARCH 62, 3603-3608 | 2002 | Neural Stem cells | IFNbeta | Adenovirus | Y |
| J of int medical Re; 40: 317-327 | 2012 | Bone Marrow MSC | IFNbeta | Adenovirus | Y |
| British Journal of Cancer; | 2013 | Bone Marrow MSC | IFNbeta | Retrovirus | Y |
| 109, 1198-1205 | | | | | |
| Stem Cell Research; 9, 270-276 | 2012 | Bone Marrow MSC | HSV1-TK | Retrovirus | Y |
| PLoS One.; 12; 10(6): | 2015 | Adipose Derived | HSV1-TK | Lentivirus | Y |
| Nanomed 10; 257-267 | 2014 | Bone Marrow MSC | HSV1-TK | Cationized pullulan | Y |
| Ann Surg; 250(5): 747-53. | 2009 | Bone Marrow MSC | HSV1-TK | Cationic Lipid | Y |
| Ann Surg; 254(5): 767-74 | 2011 | Bone Marrow MSC | HSV1-TK | Cationic Lipid | Y |
| Cancer Gene Therapy, 44-54 | 2015 | Mouse/human MSC | TRAIL | Lentivirus | Y |
| Mol Biol; 49: 904. | 2015 | Adipose Derived MSC | CDy::UPRT | Retrovirus | Y |
| J Control Release; 200: 179-87 | 2015 | Adipose Derived MSC | CDy::UPRT | Retrovirus | Y |
| Int J of Cancer; 134 (6); 1458-1465 | 2014 | Adipose Derived MSC | CDy::UPRT | Retrovirus | Y |
| Int J Cancer.; 130(10): 2455-63. | 2012 | Adipose Derived MSC | CDy::UPRT | Retrovirus | Y |
| Stem Cell Re.8 (2): 247-258 | 2010 | Adipose Derived MSC | CDy::UPRT | Retrovirus | Y |
| J of Gastroenterology and | 2009 | Adipose Derived MSC | CDy::UPRT | Cationic Lipid | N |
| Hepatology; 1393-1400 | | | | | |
| J Control Release.; 200: 179-187. | 2015 | Bone Marrow MSC | NTC, CDy::UPRT, | Cationic Lipid | Y |
| | | | HSV1-TK | ~20% efficiency | |
| Cancer Gene Therapy 25, pages285-299 | 2018 | Adipose Derived MSC | CDy::UPRT | Lentivirus | Y |
| Acta Biomater.; pii: | 2018 | Decidua-derived MSC | CDy::UPRT | ultrasound | N |
| S1742-7061(18)30660-3. | | | | nanoparticles | |
| | | | | 7% efficiency | |
| Theranostics.; 6(10): 1477-1490. | 2016 | Bone Marrow MSC | CD | Retrovirus | Y |
| PLoS One.; 12(7): e0181318. | 2017 | Bone Marrow MSC | HSV1-TK | Retrovirus | Y |

Still further examples of functional genes which may be used may include genes used for cancer therapy (Table 3) and/or genes used for still other therapeutic indications (Table 4).

TABLE 3
Therapeutic Modifications of MSCs for Cancer Therapy (adapted from
Cytotherapy, 2016, 18(11): 1435-1445, herein incorporated by reference in its entirety)
Pre-clinical studies assessing the utility of genetically modified MSCs in cancer.

| | Therapeutic | | | |
|---|---|---|---|---|
| Tumor type | modification | Cell type | Effects | Ref |
| Breast | IFN-β | BM-MSC | Reduced tumor growth and | [73] |
| | | | metastases and prolonged survival | |
| Breast | TRAIL | BM-MSC | Reduced tumor growth and metastases | [28], [89] |
| Lung | PEDF | mBM-MSC | Reduced tumor growth and prolonged survival | [113] |
| Lung | TRAIL | hUC-MSC | Prolonged survival and increased tumor apoptosis | [114] |
| Mesothelioma | TRAIL | hBM-MSC | Reduced tumor growth | [26] |
| Glioma | CDU | hAD-MSC | Tumor regression and prolonged survival | [93] |
| Glioma | HSV-tK | hAD-MSC | Reduced tumor growth | [94], [115] |
| Glioma | TRAIL | hUC-MSC | Reduced tumor growth | [22], [116] |
| Glioma | TRAIL | hBM-MSC | Inhibits tumor growth | [21] |
| HCC | Apoptin | hBM-MSC | Reduced tumor volume | [92] |
| HCC | HNF4α | hUC-MSC | Reduced tumor growth | [117] |
| HCC | IFN-β | hBM-MSC | Decreased tumor formation | [118] |
| HCC | HSV-tK | mBM-MSC | Reduced tumor growth | [103] |
| Pancreas | HSV-tK | mBM-MSC | Reduced tumor growth and metastases | [31] |
| Ascites | IL-12 | mBM-MSC | Reduced ascites volume and prolonged survival | [119] |
| Lymphoma | IL-21 | mBM-MSC | Delayed tumor development and prolonged survival | [120] |
| Prostate | IFN-β | hBM-MSC | Reduced tumor weight and prolonged survival | [121] |

TABLE 4
Genes For Modifying MSCs in Ongoing Preclinical
and Clinical Studies for Various Indications

| | | | Clinical |
|---|---|---|---|
| Gene | Publication | Indications | trial |
| GDNF, NGF | Int. J. Mol. Sci. 2014, 15, 1719-1745; | Neurodegenerative diseases, | X |
| | | Parkinson's disease | |
| Notch-1 | Stroke. 2016; 47: 1817-1824 | Stroke | Y |
| BDNF | Mol Ther. 2016 May; 24(5): 965-77 | Huntington Disease | X |
| VEGF | https://www.cirm.ca.gov/our- | Critical Limb Ischemia | Y |
| | progress/awards/phase-i-study-im- | | |
| | injection-vegf-producing-msc-treatment- | | |
| | critical-limb-ischemia-0 | | |
| α1-antitrypsin | European Respiratory | Lung disease, Chronic | Y |
| | Review 2017 26: 170044 | obstructive pulmonary disease | |
| glucagon-like | Stem Cells Transl Med. 2012 Oct; 1(10): | Post-myocardial infarction | X |
| peptide | 759-769. | (post-MI) healing | |
| bFGF | Stem Cells Transl Med. 2017 Oct; 6(10) | Bone Fracture Healing | X |

The inherent tumour tropism of MSCs [9, 10] suggests that MSCs may be utilized as cell vehicles to deliver anticancer agents specifically to tumors and their metastatic sites. A number of MSC-driven GDEPT clinical trials have presented promising results that may warrant further developments into phase II trials [7, 11]. Such approaches may facilitate localized and/or controlled conversion of the non-toxic prodrug enzymatically in close proximity to the target cells. The ‘by-stander effect’ may increase the cytotoxicity against target cells [7]. The anticancer potential of certain CD-producing MSCs has been validated in broad spectrum of solid cancers [7, 8], including gastric cancer [12-14], breast cancer [15, 16], and glioblastoma [17-19]. Preclinical studies have demonstrated that cytosine deaminase/5-fluocytosine (CD/5FC) is highly robust, where as low as 4% of CD positive cells in the tumour mass was sufficient to eradicate the tumour [20-22]. An advancement with the CD/5FC system was the inclusion of uracil phosphoribosyl-transferase (UPRT), a pyrimidine salvage enzyme that directly converts 5FU to 5-fluorouridine monophosphate (FUMP), thus bypassing the rate-limiting enzymes Dihydropyrimidine dehydrogenase (DPD) and Orotate phosphoribosyltransferase (OPRT) [23-26]. CD::UPRT/5FC may enhance the conversion of 5FC into its active metabolites by 30-1500 folds in comparison to CD/5FC and 5FU [24, 27].

In certain embodiments, the one or more functional genes may comprise a suicide gene. By way of example, in certain embodiments the one or more functional genes may comprise Cytosine Deaminase (CDy), uracil phosphoribosyltransferase (UPRT), or both. In certain embodiments, the one or more functional genes may comprise Cytosine Deaminase (CDy), uracil phosphoribosyltransferase (UPRT), herpes simplex virus-1 thymidine kinase (HSV-TK) or another thymidine kinase, or any combination thereof. In certain embodiments, the one or more functional genes may each be expressed separately, or may be expressed as a fused construct. In certain embodiments, the nucleic acid construct may comprises two or more functional genes, or the nucleic acid construct may be provided as a mixture of two or more separate nucleic acid constructs, each expressing a different functional gene of interest, for example.

In certain embodiments, the one or more functional genes may comprise a fluorescent protein or other marker or tag. In certain embodiments, the fluorescent protein may be for use in identifying and/or evaluating which MSCs were successfully transfected. In certain embodiments, the fluorescent protein may be for use in separating, isolating, selecting, or purifying transfected MSCs from non-transfected MSCs or other cells. In certain embodiments, the fluorescent protein may allow for FACs-based cell sorting to quantify, purify, or isolate transfected MSCs, for example. In certain embodiments, the one or more functional genes may comprise green fluorescent protein (GFP), for example.

In certain embodiments, the one or more functional genes of the nucleic acid construct may comprise CDy and UPRT, which may or may not be expressed as a fused construct. In certain embodiments, the one or more functional genes may further comprise a fluorescent protein such as green fluorescent protein (GFP), which may or may not be expressed as a fused construct.

In certain embodiments, the one or more functional genes of the nucleic acid construct may comprise a selection gene, such as an antibiotic resistance gene, which may be used to select for transfected MSCs, or to select for stably transfected MSCs.

While the precise number of copies in any given transfected MSC cell may vary somewhat, it is contemplated that in certain embodiments, the transfected MSC or MSCs may be each transfected with an average of at least about 1000, at least about 2000, at least about 3000, at least about 4000, at least about 5000, at least about 6000, at least about 7000, at least about 8000, at least about 9000, or at least about 10000 copies of the nucleic acid construct.

Where a plurality or population of MSCs are being transfected, it is contemplated that in certain embodiments at least about 60%, at least about 65%, at least about 70%, at least about 75%, at least about 80%, at least about 85%, at least about 90%, or at least about 95% of the MSCs may be transfected with the nucleic acid construct and express the one or more functional genes following the transfection. Accordingly, in certain embodiments where a plurality or population of MSCs are being transfected, the transfection efficiency may be any value between about 60% and about 100%, including any value therebetween rounded to the nearest 0.1, or any subrange therebetween. In certain embodiments, the transfection efficiency may be at least about 60%, at least about 65%, at least about 70%, at least about 75%, at least about 80%, at least about 85%, at least about 90%, or at least about 95%. In certain embodiments, transfection efficiency may be calculated as the % of cells expressing the one or more functional genes of interest.

In still further embodiments, it is contemplated that a cell viability of the MSCs may be at least about 70%, at least about 75%, at least about 80%, or at least about 85%. In certain embodiments, cell viability may be determined using generally any suitable technique known to the person of skill in the art having regard to the teachings herein, such as for example a propidium iodide assay as described in Cold Spring Harb Protoc. 2016 Jul. 1; 2016(7), doi: 10.1101/pdb.prot087163.

In certain embodiments, the transfected MSCs may have a multipotent phenotype which is substantially unchanged by the transfection with the nucleic acid construct. As will be understood, the transfected MSCs may be transfected with the nucleic acid construct, and the one or more functional genes encoded by the nucleic acid construct may be expressed in the transfected MSCs. In many applications, however, it is desirable that the phenotype of the transfected MSCs is otherwise substantially unchanged as compared with the MSCs prior to transfection. By way of example, MSCs have a multipotent phenotype, which may be desired for certain applications of the transfected MSCs. Accordingly, in certain embodiments, the phenotype of the transfected MSCs is multipotent and not further differentiated as compared with the MSCs pre-transfection.

In certain embodiments, the multipotent phenotype of the transfected MSCs, which may be substantially unchanged as compared with the multipotent phenotype of the MSCs pre-transfection, may comprise an immunophenotype in which the expression of CD surface markers by the MSCs is substantially unchanged after transfection. By way of example, in certain embodiments the transfected MSC or MSCs may be plastic-adherent, may express CD105, CD73, and CD90 (>95%), may lack expression of CD45, CD34, CD14, and HLA-DR surface molecules (<2%), and may be capable of differentiating into osteoblasts, adipocytes, and chondroblasts in vitro, thereby satisfying the immunophenotype criteria defined by the International Society for Cellular Therapy (ISCT) (see Cytotherapy, 2006, 8(4):315-7, and https://www.celltherapysociety.org/news/390154/FDA-Grand-Rounds-cites-ISCTs-minimal-criteria-for-defining-MSCs.htm, herein incorporated by reference in their entireties). For these purposes, it is considered in certain embodiments that the % that is acceptable for positive marker identification (i.e. for a CD marker being considered as expressed) is at least about 95% of the cells of the population post-transfection express the marker(s), and that the % that is acceptable for negative marker expression (i.e. for a CD marker being considered as not expressed) is that the population lack expression of specific marker(s) in at least 98% of the cells of the population post-transfection. For example, in certain embodiments, cells may lack expression of HLA-DR marker post-transfection, just as unmodified MSCs lack expression of this marker, indicating that phenotype is not substantially changed by transfection and that MSC quality and phenotype are not negatively changed by transfection.

Generally speaking, in certain embodiments, immunophenotype markers, or other phenotype markers, may be considered as unchanged by transfection where the expression profile of the relevant marker(s) of the transfected cells versus the expression profile of the same cells pre-transfection, or of native cells (i.e. non-modified/non-treated control cells), or of equivalent or comparable cells which have not been transfected, is substantially unchanged (i.e. a change of less than about 10%, less than about 9%, less than about 8%, less than about 7%, less than about 6%, less than about 5%, less than about 4%, less than about 3%, less than about 2%, or less than about 1%). In certain embodiments, immunophenotype markers, or other phenotype markers, may be considered as unchanged by transfection where the expression profile of the relevant marker(s) of the transfected MSC cells versus the expression profile of native MSC cells (i.e. non-modified/non-treated MSC cells) is substantially unchanged (i.e. a change of less than about 10%, less than about 9%, less than about 8%, less than about 7%, less than about 6%, less than about 5%, less than about 4%, less than about 3%, less than about 2%, or less than about 1%).

As will be understood, transfected MSCs as described herein may express one or more functional genes following transfection. Accordingly, in such embodiments, the transfected cells may be expressing one or more functional genes post-transfection, distinguishing from equivalent untransfected MSCs in this respect. As such, references herein to a multipotent phenotype which is substantially unchanged by transfection may reflect that one or more phenotypic characteristic(s) (including, but not limited to, mulipotency characteristic(s)) other than expression of the functional gene(s) may be substantially unchanged in the MSCs following transfection. Examples of phenotypic characteristics which may be substantially unchanged following transfection are described in detail herein, and may include for example any one or more of multipotency characteristic(s), immunophenotype characteristic(s), cancer tropism characteristic(s), and/or other phenotypic characteristics.

In certain embodiments, and particularly where the transfected MSCs are to be used for treatment of cancer, the multipotent phenotype of the MSCs which is substantially unchanged by transfection may include the tumor and/or cancer tropism properties of the MSC. In certain embodiments, tumor and/or cancer tropism properties of MSCs may be determined by cell invasion assay, as described in further detail in Example 1 below. In certain embodiments, tumor and/or cancer tropism properties of the transfected MSCs may be considered as unchanged where there is no substantially loss in tumor and/or cancer tropism properties following transfection (i.e. the tumor and/or cancer tropism properties may be substantially the same or increased following transfection).

In certain embodiments, the transfected MSCs may be free of virus-based transfection vehicle materials. As will be understood, virus-free transfection methods for preparing transfected MSCs as described herein are provided in detail hereinbelow. Accordingly, in certain embodiments the transfected MSCs as described herein may be free of (i.e. may not contain) virus-based transfection vehicle materials, which may include for example phage proteins and/or nucleic acids, viral membrane components, viral nucleic acids, and/or viral proteins which are typically found in virus-based gene or nucleic acid delivery approaches.

In certain embodiments, it is contemplated that the transfected MSCs described herein may be transfected with the nucleic acid construct, and may express the one or more functional genes for a period of time suitable to achieve a benefit to the subject being treated with the transfected MSCs. It has been found herein that the presently developed methods may provide transfected MSCs, including transiently transfected MSCs, which express the one or more functional genes for an extended duration of time. Accordingly, in certain embodiments, the transfected MSCs may transiently express the one or more functional genes for at least about 7, at least about 8, at least about 9, at least about 10, at least about 11, at least about 12, at least about 13, at least about 14, at least about 15, at least about 16, or at least about 17 days following transfection.

In certain embodiments, there is provided herein a population of MSC cells, or a composition comprising a plurality of MSC cells, in which there is expression MSC markers about 90% of cells or more; viability of cells as determined by standard viability test of about 80% or more; about 70% or more of MSCs being positive for the transgene as tested by flow cytometry; or any combination thereof. Preferably, for the population of MSC cells, or the composition comprising a plurality of MSC cells, there is expression MSC markers in about 90% of cells or more, viability of cells as determined by standard viability test of about 80% or more, and about 70% or more of MSCs being positive for the transgene as tested by flow cytometry.

In certain embodiments, it is contemplated that the methods described herein may be used to provide transfected MSCs that express the one or more functional genes for an extended duration of time even where transfection is transient. In certain embodiments, it is contemplated that where extended duration of expression is desirable, the nucleic acid construct may be designed to provide extended transient expression of the one or more functional genes. By way of example, in certain embodiments, it is found herein that extended duration of expression of the one or more functional genes may be achieved when the nucleic acid construct comprises a CpG-free expression plasmid. Based on these findings, the person of skill in the art having regard to the teachings herein will be aware of a variety of options for increasing duration of transient expression. In certain embodiments, it is contemplated that the nucleic acid construct may comprise a CpG-free expression plasmid or other CpG-free expression construct, a scaffold/matrix attachment region (S/MAR), an episomal vector, or an EBNA-1 containing construct. Examples of features for prolonged expression are further found in Molecular Therapy, 2006. 14(5): p. 613-626; J Biol Chem, 2000. 275(39): p. 30408-16; Nucleic acids research, 2014. 42(7): p. e53-e53; and DOI:https://doi.org/10.1016/j.ymthe.2006.03.026, each of which is herein incorporated by reference in its entirety. In certain embodiments, some or all of these features may be implemented in the design of nucleic acid constructs as described herein. In certain embodiments, some or all of these features may be implemented as modules which may be added to nucleic acid constructs as described herein. For example, in certain CD::UPRP:GFP constructs as described and used herein, features/modules of CpG-free and S/MAR were used in the nucleic acid constructs.

In certain embodiments, the transfected MSCs may be produced by any of the methods as described herein. By way of example, in certain embodiments, the transfected MSCs may be transfected with the nucleic acid construct using a cationic polymer, a first agent capable of redirecting endocytosed nucleic acids from intracellular acidic compartments, and a second agent capable of stabilizing a microtubular network of the MSCs. Further description of such methods and components is provided hereinbelow. By way of example, in certain embodiments, the cationic polymer may comprise linear or branched polyethylenimine (PEI); the first agent may comprise 1,2-Dioleoyl-sn-glycero-3-phosphoethanolamine (DOPE)/cholesteryl hemisuccinate (CHEMS) (DOPE/CHEMS); and/or the second agent may comprise a histone deactylase inhibitor (HDACi) such as SAHA (Vorinostat).

In certain embodiments, such as where the functional genes comprise one more of Cytosine Deaminase (CDy), uracil phosphoribosyltransferase (UPRT), or herpes simplex virus-1 thymidine kinase (HSV-TK), the MSCs may be sensitive to treatment with 5-fluorocytosine (5FC) or ganciclovir (GCV). In certain embodiments, the transfected MSCs may be capable of converting: a) 5FC to 5-fluorouridine (5FU), 5-fluorouridine monophosphate (FUMP), or both; b) ganciclovir to ganciclovir monophosphate; or c) a combination of a) and b). In certain embodiments, the MSCs may be for use in treating cancer. In certain embodiment, the transfected MSCs may be for use in combination with 5FC, 5FU, GCV, or or any combination thereof.

In certain embodiments, the transfected MSCs may be substantially undifferentiated.

In still another embodiment, there is provided herein a method for transfecting mesenchymal stem cells (MSCs) with a nucleic acid construct from which one or more functional genes are expressed, the method comprising:
exposing the MSCs to a transfection mixture comprising the nucleic acid construct which is complexed with a cationic polymer;
exposing the MSCs to a first agent capable of redirecting endocytosed nucleic acids from intracellular acidic compartments and a second agent capable of stabilizing a microtubular network of the MSCs; and incubating the MSCs;

thereby providing MSCs transfected with the nucleic acid construct.

Examples of suitable MSCs, nucleic acid constructs, and functional genes are already described in detail herein. By way of example, in certain embodiments the one or more functional genes may comprise a suicide gene; Cytosine Deaminase (CDy) and/or thymidine kinase (TK); uracil phosphoribosyltransferase (UPRT); both CDy and UPRT which may or may not be provided as a fused construct; a fluorescent protein such as green fluorescent protein (GFP); CDy, UPRT, and GFP, which may or may not be provided as a fused construct; herpes simplex virus-1 thymidine kinase (HSV-TK); or any combinations thereof.

In certain embodiments, the MSCs may be derived from cord blood, neonatal birth-associated tissue, Wharton's jelly, umbilical cord, cord lining, placenta, or other source of MSC cells. In certain embodiments, the MSCs may be adipose tissue-derived MSC (AT-MSC), bone marrow-derived MSC (BM-MSC), or umbilical cord-derived MSC (UC-MSC). In another embodiment, the MSCs may be sourced from human, canine, feline, equine, or other species.

In certain embodiments, the nucleic acid construct may comprise a CpG-free expression plasmid or other CpG-free expression construct, a scaffold/matrix attachment region (S/MAR), an episomal vector, or an EBNA-1 containing construct.

In certain embodiments, cationic polymers may comprise any suitable cationic or polycationic or partially cationic polymer which complexes with the nucleic acid construct and is capable of delivering the nucleic acid construct into the MSCs upon exposure thereto. In certain embodiments, the cationic polymer may be selected from polyethylene imine, polycationic amphiphiles, DEAE-dextran, cationic polymers, their derivatives, or any combinations thereof. In certain embodiments, the cationic polymer may comprise a cationic polymer such as a dendimer, branchedpolyethylenimine (BPEI), linear-polyethylenimine (LPEI), Poly(amidoamine) (PAMAM), XtremeGENE HP®, or any combinations thereof. In certain embodiments, the cationic polymer may comprise LPEI. In certain embodiments, the cationic polymer may be a homopolymer, a co-polymer, or a block-co-polymer, for example. In certain embodiments, the cationic polymer may have a size of about 5 kDa to about 200 kDa. In certain embodiments, the cationic polymer may have a size of equal to or less than about 5 kDa. In certain embodiments, the cationic polymer may have a size of equal to or more than about 200 kDa. In certain embodiments, the cationic polymer may comprise linear or branched polyethylenimine (PEI) poly(amidoamine) PAMAM, or another cationic polymer, or any combinations thereof. In certain embodiments, the cationic polymer may comprise linear polyethylenimine (LPEI).

In certain embodiments, the amount of nucleic acid construct in the transfection mixture to which the MSCs are exposed may be between about 200 to about 500 ng per 1.9 cm² surface area. In certain embodiments, the amount of nucleic acid construct in the transfection mixture to which the MSCs are exposed may be between about 250 to about 400 ng per 1.9 cm² surface area. In certain embodiments, the amount of nucleic acid construct in the transfection mixture to which the MSCs are exposed may be between about 300 to about 350 ng per 1.9 cm² surface area. In certain embodiments, the amount of nucleic acid construct to which the MSCs are exposed may be any value rounded to the nearest 0.1 between about 200 to about 500 ng per 1.9 cm², or any subrange therebetween.

In certain embodiments of the any of the above methods, a ratio of cationic polymer to nucleic acid construct may be about 1 μg to about 30 μg cationic polymer per 1 μg of nucleic acid construct in the transfection mixture, or any value rounded to the nearest 0.1 therebetween, or any subrange therebetween.

In certain embodiments, the cationic polymer and nucleic acid N/P may range from about 5 to about 100, for example about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99, or 100, or any value rounded to the nearest 0.1 therebetween, or any subrange therebetween.

In certain embodiments, the transfection mixture may comprise a complexing buffer, a cell media or cell buffer, or any combination thereof.

In certain embodiments, the first agent capable of redirecting endocytosed nucleic acids from intracellular acidic compartments may comprise any suitable agent capable of directing genetic material away from a non-productive acidic compartment of the cell. In another embodiment, the first agent may comprise a lipid, a peptide fusogenic agent, or a combination thereof. In certain embodiments, the first agent may comprise DOPE, CHEMS, DPPC or DOPC, or any combinations thereof. In certain embodiments, the first agent may comprise haemagglutinin (HA2-peptide), influenza-derived fusogenic peptide diINF-7, T domain of Diphtheria toxin, or polycationic peptides, such as polylysine and/or polyarginine, or any combinations thereof. In certain embodiments, the first agent may comprise 1,2-Dioleoyl-sn-glycero-3-phosphoethanolamine (DOPE)/cholesteryl hemisuccinate (CHEMS) (DOPE/CHEMS). Various ratios of DOPE: CHEMs may be used, for example in certain embodiments a ratio between about 9:1 and 1:9 may be used, such as a ratio of about 9:1, 8:2, 7:3, 6:4, 5:5, 4:6. In certain embodiments, a mixture of a fusogenic lipid and a helper lipid may be used, such as a mixture of DOPE and CHEMS. In certain embodiments, three lipids may be used. For example, in certain embodiments a mixture of DPPC:DOPE:CHEMS, in various ratios, may be used.

In certain embodiments, the second agent capable of stabilizing a microtubular network of the MSCs may comprise any suitable agent which stabilizes the microtubule or a network thereof. In certain embodiments, the second agent may be capable of enhancing tubulin acetylation. In certain embodiments, the second agent may be selected from a histone deacetylase inhibitor (HDACi), such as a histone deactylase 6 inhibitor (HDAC6i), a tubulin binding agent (TBA) and siRNA that is capable of directly or indirectly affecting the microtubule network stability. In certain embodiments, the HDACi may comprise Tubastatin A, belinostat, bufexamac, panobinostat, PCI-24781, SAHA (vorinostat), scriptaid, trichostatin A, valporic acid, B2, salermide, sirtinol, or any combinations thereof. In certain embodiments, the second agent may comprise a histone deactylase inhibitor (HDACi), such as SAHA (Vorinostat).

In certain embodiments, the first and second agents may together form TrafEn™, which stands for trafficking enhancer for directing genetic material or complex containing genetic material to a productive pathway for efficient transfection.

Examples of suitable first agents capable of redirecting endocytosed nucleic acids from intracellular acidic compartments, and suitable second agents capable of stabilizing a microtubular network of the MSCs, and TrafEN™, are described in detail in WO2014/070111 entitled A Novel Reagent for Gene-Drug Therapeutics, and in Ho Y. K., et al., Enhanced Non-Viral Gene Delivery by Coordinated Endosomal Release and Inhibition of β-Tubulin Deactylase, Nucleic Acids Research, 2017, 45(6): e38, both of which are herein incorporated by reference in their entireties.

In certain embodiments of the above methods, the MSCs are not centrifuged during exposure to the transfection mixture, to the first agent and second agent, during incubation, or any combination thereof. In certain embodiments of the above methods, the step of incubating the MSCs may optionally comprise gentle mixing without centrifugation.

In certain embodiments, centrifugation may be used to help rapidly deposit polymer-complexed DNA onto cells. The transfection mixture may then be removed immediately or shortly after centrifugation, so as to minimize toxicity of free cationic polymer (which is not substantially spun down by the centrifugation) to the cells. Accordingly, in certain embodiments, particularly where smaller scales readily amenable to centrifugation are being used, centrifugation may be used for transfection of the cells. By way of example, a centrifugation approach may include steps of adding transfection mixture to cells; centrifuging to deposit nucleic acid complexes on cells; removing the transfection mixture to remove free polymer from contact with the cells; and replacing with fresh media which may include TrafEn, or to which TrafEn may be added, for example.

In certain embodiments, it may be desirable to operate on a larger scale, such as in veterinary and/or human therapy indications. At such larger scale, centrifugation may be undesirable. Centrifugation may be undesirable in other instances as well, such as applications where centrifugation equipment is unavailable, inconvenient, and/or costly, for example. As described in detail herein, it has now been found that in certain embodiments, centrifugation may be omitted. When centrifugation is omitted, incubation time may be extended to, for example, about 2 to about 24 hours in certain embodiments in order to sufficiently contact cells with the polymer-complexed DNA. The presence of free polymer during this incubation time may be toxic, depending on cells and polymer being used. Accordingly, in certain embodiments selection of cationic polymer may be adjusted appropriately for the particular cell type and incubation time so as to reduce or avoid toxicity to cells, as described in detail herein.

As described in detail herein, methods described herein, which may or may not use centrifugation, may provide high transfection efficiencies (>about 70%, for example).

In embodiments omitting centrifugation, the methods may be more readily scalable to accommodate larger production scales for preclinical and/or clinical trials, for example. However, the present inventors have found that when omitting centrifugation during transfection, incubation time during transfection may extended in order to achieve high transfection efficiencies. Accordingly, in certain embodiments such as those omitting centrifugation, the step of incubating the MSCs may comprise incubating the MSCs for at least about 2 hours. In certain embodiments, the step of incubating the MSCs may comprise incubating the MSCs for about 2 hours to about 24 hours, or for about 4 hours to about 18 hours, or any value between 2 and 24 hours rounded to the nearest 0.1, or any subrange therebetween.

The present inventors have further identified in embodiments where centrifugation is omitted and/or where incubation time is extended, selection of cationic polymer may be adjusted appropriately since certain cationic polymers can cause toxicity which may be undesirable particularly where incubation times are extended. Furthermore, different types of MSCs (i.e. variations in type, source, cell line, and growth conditions) may exhibit different tolerances toward cationic polymer and/or extended incubation periods. Accordingly, in certain embodiments, the method of transfection may be tailored to the particular MSCs being used. Example 2 below sets out some examples where DNA amounts/conditions and cationic polymer selection was performed to avoid toxicity during transfection of certain MSCs and obtain high transfection efficiency. Accordingly, in certain embodiments of the methods described herein, the cationic polymer may comprise a cationic polymer which has been identified as having low cytotoxicity against the MSCs of the particular application. In certain embodiments, cationic polymers may be screened by size and/or number of charges, for example. In certain embodiments, certain larger cationic polymers may be preferential for some cells but may be somewhat toxic to others, for example. In some cases, smaller cationic polymers and/or less charged cationic polymers may typically be less toxic, but may exhibit low transfection efficiency and/or rate in certain cell lines. In certain embodiments, TrafEn may be used to boost transfection efficiency, for example.

In certain embodiments, buoyant density of the media, which may vary between cell types, may be considered when selecting cationic polymer and/or polymer-DNA complexes, since this may have an effect on deposit rate of the polymer-DNA complexes on the cells. In certain embodiments, complexes and/or media may be selected to favor good depositing on cells, and/or cationic polymer may be selected such that free cationic polymer in non-toxic, or has low toxicity, toward the particular cells.

In certain embodiments, it is contemplated that cationic polymers may be screened to identify those providing suitable transfection efficiency and/or cell viability for the particular MSCs of interest, as these are two notable features identified herein for determining the level of compatibility of a cationic polymer with a particular MSC type/donor for providing efficient transfection without centrifugation. In certain embodiments, the cationic polymer may be selected such that it does not cause appreciable or detrimental levels of cytotoxicity during an incubation period of at least about 2 hours, or about 4 hours, for example. If the cationic polymer is non toxic to the cells, the incubation period may be allowed to proceed for a longer time. In certain embodiments, toxicity may be evaluated by any suitable method, such as propidium iodide assay. In certain embodiments, the cell viability (or cell viability target) may be equal to or greater than about 70% post-transfection.

In certain embodiments of any of the above method or methods, the step of exposing the MSCs to the transfection mixture may comprise complexing the nucleic acid construct with the cationic polymer so as to provide the transfection mixture comprising complexed nucleic acid construct, and adding the transfection mixture to the MSCs. In other words, the step of exposing the MSCs to the transfection mixture will preferably comprise pre-complexing or combining the nucleic acid construct and the cationic polymer prior to addition to the MSCs.

In another embodiment of any of the above method or methods, the step of exposing the MSCs to the transfection mixture may comprise adding the transfection mixture to the MSCs and incubating the MSCs with the transfection mixture.

In another embodiment of any of the above method or methods, the step of exposing the MSCs to the first and second agents may comprise adding the first and second agents together with, or immediately after, adding or exposing the MSCs to the transfection mixture. In certain embodiments, this may be performed where centrifugation is omitted.

In another embodiment of any of the above method or methods, the step of exposing the MSCs to the first and second agents may comprise adding the first and second agents together with the transfection mixture in the step of exposing the MSCs to the transfection mixture, or may comprise adding the first and second agents to the MSCs already being contacted with the transfection mixture (i.e. the transfection mixture may not be removed before the first and second agents are added). In certain embodiments, this may be performed where centrifugation is omitted.

In still another embodiment of any of the above method or methods, the step of exposing the MSCs to the first and second agents may comprise replacing the transfection mixture with cell culture media supplemented with the first agent and second agent. In certain embodiments, this may be performed where centrifugation is used to help rapidly deposit polymer-complexed DNA onto cells, so as to reduce free polymer toxicity to the cells. In certain embodiments, the cell culture media may comprise complete media.

In certain embodiments of any of the above method or methods, the MSCs may be at about 60% confluency, and the MSCs may be seeded for about 24 hours prior to exposure to the transfection mixture.

In certain embodiments of any of the above method or methods, the transfection mixture may comprise the complexed nucleic acid construct in serum free DMEM, or in fresh culture media.

In still further embodiments of any of the above method or methods, the step of exposing the MSCs to the transfection mixture may comprise adding the transfection mixture (which may or may not further comprise fresh culture media) to the cells, without removing a culture/growth media from the cells before adding the transfection mixture. In certain embodiments, this may be performed where centrifugation is omitted.

In certain embodiments of the above method or methods, the step of exposing the MSCs to the transfection mixture may comprise:

optionally, replacing a culture/growth media in which the cells are being cultured with fresh culture/growth media; and

adding the transfection mixture (which may or may not further comprise fresh culture media, or which may be added simultaneously or sequentially with fresh culture media) to the cells, without removing the culture/growth media from the cells before adding the transfection mixture (if present).

Accordingly, in certain embodiments it is contemplated that cell culture/growth media may be replaced or refreshed prior to addition of the complexed nucleic acid construct to the cells, so as to provide fresh culture media before transfection is performed. In certain embodiments, this may be performed where centrifugation is omitted.

In still further embodiments of any of the above method or methods, the step of exposing the MSCs to the transfection mixture may comprise removing a culture media from the MSCs and replacing the culture media with the transfection mixture. In certain embodiments, this may be performed where centrifugation is used to help rapidly deposit polymer-complexed DNA onto cells.

In certain embodiments, it is contemplated that the step of exposing the MSC to the transfection mixture may comprise incubating the MSCs with the transfection mixture under mild centrifugation. For example, where the method is performed at small scale and/or where suitable centrifugation apparatus is available, it is contemplated that centrifugation may be performed in certain embodiments. Mild centrifugation may be performed to avoid toxicity of free polymer following addition of the polymer-nucleic acid construct. In certain embodiments, the method may comprise adding the transfection mixture to the cell culture, performing mild centrifugation (for example, for about 5 minutes) to deposit polymer-nucleic acid construct complexes onto the cells), and removing the transfection mixture (containing free polymer, which is relatively small and is not substantially spun down by the centrifugation). In certain embodiments, the mild centrifugation may comprise about 200 g for about 5 minutes.

In certain embodiments of the method or methods described herein, the transfection may be carried out in a flat bed vessel, for example, in which the amount of reagent is increased, the cell density is tuned to this increased amount, and the amount of DNA is increased according to the surface area of the cell culture vessel.

In certain embodiments of the method or methods described herein, the MSCs may be cultured on microcarriers (for example, microbeads), and may thus be suspended, during transfection, optionally while under shaking or other agitation. In certain embodiments, the microcarrier may comprise a microbead. In certain embodiments, the microcarrier may comprise a Type 1 porcine collagen coated microcarrier. In certain embodiments, the microcarrier may comprise Cytodex® 3. In certain embodiments where a microcarrier is used, the transfection may be performed under shaking and increased cell density, which may allow for larger-scale production. In addition, the rpm during shaking may be adjusted according to the type of vessel and density/number of the microcarrier used.

Other approaches with flat bed vessels and microcarriers will follow similar steps, for example: Step 1→expose MSCs to a transfection mixture comprising the nucleic acid construct which is complexed with a cationic polymer; Step 2→expose the MSCs to a first agent capable of redirecting endocytosed nucleic acids from intracellular acidic compartments and a second agent capable of stabilizing a microtubular network of the MSCs; and Step 3→incubating the MSCs, thereby providing the MSCs transfected with the nucleic acid construct. The number of cells added per surface area cm2 and the cell culture volume to use is adjusted for each vessel. For microcarrier applications, the shaking speed will be adjusted to prevent microcarrier aggregation.

In certain embodiments of the method or methods described herein, the step of incubating the MSCs during transfection may comprise rotating bioreactor-type agitation (for example, rotating Erlenmeyer flask), wave bioreactor, rotating wall bioreactor, stirred tank bioreactor, or shaker-type agitation for at least a portion of the incubation time (see FIG. 52 for further examples).

In certain embodiments of any of the above method or methods, the one or more functional genes may be transiently expressed in the transfected MSCs, may be stably transfected in the transfected MSCs, or a combination thereof.

In certain embodiments of any of the above method or methods, the transfected MSCs may be each transfected with an average of at least about 1000, at least about 2000, at least about 3000, at least about 4000, at least about 5000, at least about 6000, at least about 7000, at least about 8000, at least about 9000, or at least about 10000 copies of the nucleic acid construct

In certain embodiments of the above method or methods, a multipotent phenotype of the transfected MSCs may be substantially unchanged by the transfection. In certain embodiments, the multipotent phenotype may comprise tumor and/or cancer tropism properties of the MSC. In certain embodiments, the multipotent phenotype may comprise an immunophenotype in which the expression of CD surface markers may be substantially unchanged after transfection. In certain embodiments, the transfected MSCs may be plastic-adherent, may express CD105, CD73, and CD90 (>95%), may lack expression of CD45, CD34, CD14, and HLA-DR surface molecules (<2%), and may be capable of differentiating into osteoblasts, adipocytes, and chondroblasts in vitro, satisfying the immunophenotype criteria defined by the International Society for Cellular Therapy (ISCT). Phenotype of the transfected cells is already described in detail hereinabove, and in the Examples below.

In certain embodiments of any of the above method or methods, at least about 60%, at least about 65%, at least about 70%, at least about 75%, at least about 80%, at least about 85%, at least about 90%, or at least about 95% of the MSCs may be transfected with the nucleic acid construct and express the one or more functional genes.

In certain embodiments of any of the above method or methods, a cell viability of the transfected MSCs may be at least about 70%, at least about 75%, at least about 80%, or at least about 85%.

In certain embodiments of any of the above method or methods, the transfected MSCs may be undifferentiated.

In certain embodiments of any of the above method or methods, the method may be free of virus-based transfection vehicle materials.

In certain embodiments of any of the above method or methods, the MSCs may transiently express the one or more functional genes for at least about 7, at least about 8, at least about 9, at least about 10, at least about 11, at least about 12, at least about 13, at least about 14, at least about 15, at least about 16, or at least about 17 days following transfection.

In certain embodiments, the resultant MSCs may be sensitive to treatment with 5-fluorocytosine (5FC) or ganciclovir (GCV) or both. In certain embodiments, the resultant MSCs may convert: a) 5FC to 5-fluorouridine (5FU), 5-fluorouridine monophosphate (FUMP) or both; b) ganciclovir to ganciclovir monophosphate; or c) a combination of a) and b).

In certain embodiments, the one or more functional genes may comprise a fluorescent protein, and the method may further comprise a step of isolating, selecting, or purifying the transfected MSCs using cell sorting or FACS. This step may be performed, for example, where particularly high purity is desired. As will be understood, such isolating, selecting, or purifying may be optional, since in clinical application for example it is contemplated that a population which is about >70% positive for the therapeutic gene may be acceptable, and as described herein may be obtained without further steps of isolating, selecting, or purifying in certain embodiments.

In still another embodiment of the above method or methods, there is provided herein a method for transfecting mesenchymal stem cells (MSCs) with a nucleic acid construct from which one or more functional genes are expressed, the method comprising:
culturing the MSCs in a growth medium;
adding a transfection mixture comprising the nucleic acid construct which is complexed with a cationic polymer to the MSCs without removing the growth medium from the MSCs;
adding a first agent capable of redirecting endocytosed nucleic acids from intracellular acidic compartments and a second agent capable of stabilizing a microtubular network of the MSCs to the MSCs; and
incubating the MSCs while in contact with all of the transfection mixture, the first agent, and the second agent for an incubation period;
   wherein the first and second agents are added to the MSCs simultaneously with the addition of the transfection mixture, sequentially with the addition of the transfection mixture, or in combination with the transfection mixture; and
   wherein the MSCs are not centrifuged between the adding of the transfection mixture and expiry of the incubation period;
   thereby providing MSCs transfected with the nucleic acid construct.

In certain embodiments, the step of culturing the MSCs in a growth media may comprise providing the cells with fresh growth medium (i.e. replacing a spent or partially spent growth medium with fresh growth medium, or adding fresh growth medium to a spent or partially spent growth medium.

In certain embodiments, the incubation period may be at least about 2 hours.

In certain embodiments, the incubation period may be about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, or about 36 hours.

In certain embodiments of any of the above method or methods, the method may produce any of the transfected MSCs as described herein.

In still another embodiment, there is provided herein an MSC, or plurality of MSCs, produced by any of the method or methods described herein.

In another embodiment, there is provided herein a composition comprising any of the MSC or MSCs as described herein, and at least one of a pharmaceutically acceptable carrier, diluent, excipient, cell media, or buffer.

In certain embodiments, a pharmaceutically acceptable carrier, diluent, excipient, cell media, or buffer may include any suitable PBS buffer, cryopreservative media, matrigel, or hydrogel, for example. In certain embodiments, there is provided herein a composition comprising a suspension of MSCs as described herein in PBS or another buffer or cell media. In another embodiment, there is provided herein a composition comprising MSCs as described herein frozen with a cryopreservative media.

In still another embodiment, there is provided herein a theranostic agent comprising any of the MSC or MSCs described herein. By way of example, in certain embodiments, the theranostic agent may comprise an MSC expressing both a therapeutic or suicide gene, and a fluorescent protein. The MSCs may have cancer and/or tumor tropism properties, and may be used to indication location of cancer or tumor cells by way of fluorescence, at which point prodrug may be added (where a suicide gene is used) to result in an anti-cancer or anti-tumor effect, for example.

In still another embodiment, there is provided herein a kit for transfecting a mesenchymal stem cell (MSC) with a nucleic acid construct from which one or more functional genes are transiently expressed, the kit comprising one or more of:
an MSC;
a nucleic acid construct designed for transient expression of one or more functional genes;
a cell culture media;
a cationic polymer;
a first agent capable of redirecting endocytosed nucleic acids from intracellular acidic compartments;
a second agent capable of stabilizing a microtubular network of the MSC;
instructions for performing a method as described herein;
5FC;
GCV; and/or
5FU.

In certain embodiments, MSC may be any MSC as described herein. In certain embodiments, the MSC may be derived from cord blood, neonatal birth-associated tissue, Wharton's jelly, umbilical cord, cord lining, placenta, or other source of MSC cells. In certain embodiments, the MSC may comprise an adipose tissue-derived MSC (AT-MSC), bone marrow-derived MSC (BM-MSC), or umbilical cord-derived MSC (UC-MSC). In another embodiment, the MSCs may be sourced from human, canine, feline, equine, or other species.

In certain embodiments, the nucleic acid construct may be any nucleic acid construct as described herein, and the one or more functional genes may be any one or more function genes as described herein. In certain embodiments, the nucleic acid construct may comprise a CpG-free expression plasmid or other CpG-free expression construct, a scaffold/matrix attachment region (S/MAR), an episomal vector, or an EBNA-1 containing construct.

In certain embodiments, the cationic polymer may comprise any cationic polymer as described herein. In certain embodiments, the cationic polymer may comprise linear or branched polyethylenimine (PEI), poly(amidoamine) PAMAM, or another cationic polymer. In certain embodiments, the cationic polymer may comprise linear polyethylenimine (LPEI). In certain embodiments, the cationic polymer may comprise a cationic polymer which has been identified as having low cytotoxicity against the MSCs. In certain embodiments, the cationic polymer may have a size of about 5 kDa to about 200 kDa.

In certain embodiments, the first agent may comprise any suitable first agent as described herein. In certain embodiments, the first agent may comprise one or more of DOPC, DPPC, or another fusogenic lipid. In certain embodiments, the first agent may comprise 1,2-Dioleoyl-sn-glycero-3-phosphoethanolamine (DOPE)/cholesteryl hemisuccinate (CHEMS) (DOPE/CHEMS), 1,2-dipalmitoyl-sn-glycero-3-phosphocholine (DPPC) or another fusogenic lipid, or any combinations thereof.

In certain embodiments, the second agent may comprise any suitable second agent as described herein. In certain embodiments, the second agent may comprise a histone deactylase inhibitor (HDACi) such as SAHA (Vorinostat).

In certain embodiments, the one or more functional genes may comprise a suicide gene; Cytosine Deaminase (CDy); thymidine kinase (TK); uracil phosphoribosyltransferase (UPRT); both CDy and UPRT, which may or may not be provided as a fused construct; a fluorescent protein such as green fluorescent protein (GFP); CDy, UPRT, and GFP, which may be provided as a fused construct; herpes simplex virus-1 thymidine kinase (HSV-TK); or any combinations thereof.

In certain embodiments, a ratio of cationic polymer to nucleic acid construct in the kit may be about 1 μg to about 30 μg cationic polymer per 1 μg of nucleic acid construct.

In certain embodiments, the kit may be for preparing an MSC-based anti-cancer agent. In certain embodiments, the kit may further comprise instructions and/or apparatus for performing a method for treating cancer as described herein. In certain embodiments, a syringe or other suitable injection device may be provided for intratumoral or intravenous or subcutaneous injection or infusion of MSCs. In certain embodiments, MSCs may be embedded with a biomaterial, such as gelfoam, for administration.

In certain embodiments, there are provided herein methods for scalable non-viral gene modification of Mesenchymal Stem cells (MSC) for cancer treatment. In certain embodiments, methods as described herein may include transfecting MSC with one or more suicide genes in the presence of a formulation of transfection enhancer (TrafEn). In certain embodiments, such methods may comprise using a first agent capable of redirecting endocytosed nucleic acids from intracellular acidic compartments and a second agent capable of stabilizing the microtubular network thereof. In certain embodiments, high efficiency modification in number and expression of modified cells may provide for the generation of high potency MSCs expressing therapeutic genes, for example the suicide gene Cytosine Deaminase (CD). In certain embodiments, the modified MSCs may be administered to subjects with a tumor or cancer. In certain embodiments, the therapeutic gene expressed by the MSCs may convert a prodrug to a toxic agent that reduces or eliminates tumour bulk. In certain embodiments, the methods described herein may be used in the manufacture of a medicament for treating cancers and/or other indications. Also described herein are methods for delivering a genetic material into a cell, and kits therefore. In certain embodiments, the MSCs may be modified with generally any suitable cancer targeting therapeutic gene(s), and/or generally any other suitable therapeutic gene(s) for treatment of generally any other suitable diseases and/or disorders.

Uses and Methods of Treating Diseases or Disorders Such as Cancer Using Transfected Mesenchymal Stem Cells

As described in detail herein, transfected MSCs and methods and kits for preparing transfected MSCs are provided, wherein the transfected MSCs may express one or more functional genes. In certain embodiments, the one or more functional genes may comprise one or more therapeutically active genes, producing one or more therapeutically active RNAs, peptides, polypeptides, or proteins for example. As will be understood, the MSCs described herein may therefore be for use in treating, preventing, or ameliorating generally any disease or disorder toward which the one or more functional genes are therapeutically active. The following discussions mainly relate to the treatment of cancer, however the skilled person having regard to the teachings herein will recognize that a variety of other diseases or disorders are also contemplated herein.

In an embodiment, there is provided herein a use of any of the MSC or MSCs as described herein for killing a cancer cell.

In an embodiment, there is provided herein a use of any of the MSC or MSCs as described herein for treating cancer in a subject in need thereof.

In certain embodiments, cancer may include any one or more of various types of solid tumors (such as tyroid carcinoma, sacarma, lymphoma, squamous cancer, others). As MSCs may exhibit strong tropism, it is contemplated that the location of the cancer may be generally anywhere and location may not present a significant issue. Furthermore, in certain embodiments, it is contemplated that MSCs and treatments as described herein may be tailored to a particular cancer, and that MSC prodrug strategies as described herein may be generally agnostic to cancer type.

In certain embodiments, the cancer may comprise lymphoma, clear cell carcinoma, glioblastoma, temozolomide resistant glioblastoma, perianal carcinoma, oral melanoma, thyroid carcinoma, soft tissue carcinoma, cancer ulceration, nasal tumor, or gastrointestinal cancer, or any combinations thereof, for example.

In certain embodiments, the subject may comprise a vertebrate animal, a mammal, or a human.

In certain embodiments, the MSCs may be for use combination with (either simultaneously, sequentially, or mixed with) one or more additional drugs or therapeutics active against the disease or disorder to be treated, such as one or more anti-cancer drugs where the disease or disorder is cancer.

In certain embodiments, particularly where the one or more functional genes expressed by the transfected MSCs comprise a suicide gene or express an enzyme which converts a prodrug to an active form, the MSCs may be for use in combination with (either simultaneously, sequentially, or mixed with) one or more corresponding prodrugs. By way of example, in certain embodiments, the one or more functional genes may comprise Cytosine Deaminase (CDy); thymidine kinase (TK); uracil phosphoribosyltransferase (UPRT); both CDy and UPRT, which may or may not be provided as a fused construct; herpes simplex virus-1 thymidine kinase (HSV-TK); or any combination thereof, and may be for use in combination with 5FC, 5FU, GCV, or any combination thereof.

In another embodiment, there is provided herein a use of any of the MSC or MSCs as described herein, in the manufacture of a medicament for the treatment of cancer. In certain embodiments, the MSC or MSCs may be for use in combination with 5FC, 5FU, GCV, or any combination thereof.

In certain embodiments, the MSC or MSCs as described herein may be for administration to the subject via generally any suitable technique appropriate for the subject and/or the disease to be treated. By way of example, in certain embodiments, it is contemplated that the MSCs may be administered to the subject systemically (for example, by intravenous injection), or locally (for example, by local injection or implantation). In certain embodiments, MSCs may be administered intravenously as described in, for example, Oncotarget. 2017 Oct. 6; 8(46): 80156-80166, or by intracranial administration as described in, for example, Clin Cancer Res. 2017 Jun. 15; 23(12):2951-2960, each of which are herein incorporated by reference in their entireties. In certain embodiments, the MSC or MSCs as described herein may be for administration to the subject by intraportal, intraperitoneal, intravenous, intratumoral, subcutaneous, intracranial injection or infusion, or administration embedded in a hydrogel or gel foam for administration or implantation to the subject.

In still another embodiment, there is provided herein a method for treating cancer in a subject in need thereof, said method comprising:
administering MSC or MSCs as described herein to a region in proximity with a cancer cell of the subject,
wherein the one or more functional genes in the MSC or MSCs contribute to an anticancer effect on the cancer cell.

In certain embodiments, the subject may comprise a vertebrate animal, a mammal, or a human.

In certain embodiments, the cancer may comprise lymphoma, clear cell carcinoma, glioblastoma, temozolomide resistant glioblastoma, perianal carcinoma, oral melanoma, thyroid carcinoma, soft tissue carcinoma, cancer ulceration, nasal tumor, or gastrointestinal cancer, or any combinations thereof, for example.

In certain embodiments, the MSC or MSCs as described herein may be administered to the subject via generally any suitable technique appropriate for the subject and/or the disease to be treated. By way of example, in certain embodiments, it is contemplated that the MSCs may be administered to the subject systemically (for example, by intravenous injection), or locally (for example, by local injection, intratumoral injection, subcutaneous injection, or implantation or infusion). In certain embodiments, MSCs may be administered intravenously as described in, for example, Oncotarget. 2017 Oct. 6; 8(46): 80156-80166, or by intracranial administration as described in, for example, Clin Cancer Res. 2017 Jun. 15; 23(12):2951-2960, each of which are herein incorporated by reference in their entireties. In certain embodiments, the MSC or MSCs as described herein may be for administration to the subject by intravenous, intratumoral, subcutaneous, intracranial injection or infusion, or administration embedded in a hydrogel or gel foam for administration or implantation to the subject.

As will be understood, in certain embodiments the MSCs may be administered to the subject such that cells associated with the disease or disorder (such as cancer or tumor cells) are contacted with, or in suitable proximity with the MSCs such that the one or more functional genes expressed by the MSCs may exert a therapeutic effect (directly, or indirectly via (for example) prodrug conversion) on the cells associated with the disease or disorder. In certain embodiments, the MSCs may have tropism properties for the cells associated with the disease or disorder, assisting with the positioning of the MSCs in suitable proximity with the cells associated with the disease or disorder. In certain embodiments, MSCs may be administered directly in contact with the cells associated with the disease or disorder, or within a proximity of about 1-3 cm from the cells associated with the disease or disorder. In certain embodiments, since prodrug therapy may result in bystander effect, direct contact of MSCs with disease cells such as cancer cells may be unnecessary.

In certain embodiments, particularly where the one or more functional genes expressed by the transfected MSCs comprise a suicide gene or express an enzyme which converts a prodrug to an active form, the MSCs may administered in combination with (either simultaneously, sequentially, or mixed with) one or more corresponding prodrugs. By way of example, in certain embodiments, the one or more functional genes may comprise Cytosine Deaminase (CDy); thymidine kinase (TK); uracil phosphoribosyltransferase (UPRT); both CDy and UPRT, which may or may not be provided as a fused construct; herpes simplex virus-1 thymidine kinase (HSV-TK); or any combination thereof, and may be for use in combination with 5FC, 5FU, GCV, or any combination thereof.

In certain embodiments, the one or more functional genes of the MSCs and the prodrug may be designed such that conversion of the prodrug to active form may target or kill the MSCs in addition to the surrounding cells associated with the disease or disorder (such as cancer or tumour cells, for example). Accordingly, in certain embodiments where the MSCs are to be used in combination with another drug such as a prodrug, it may be desirable to maintain the MSCs and prodrug separate from one another until the MSCs are first introduced to the appropriate cells of the subject, such that the MSCs are not killed or deactivated before they can provide a therapeutic effect. In certain embodiments, MSCs may take at least some time to move toward the tumor by tropism, and so it is contemplated that in certain embodiments, administration of the prodrug may be delayed until the MSCs are near the tumors, for example.

In certain embodiments, the MSC or MSCs may transiently express the one or more functional genes for at least about 7, at least about 8, at least about 9, at least about 10, at least about 11, at least about 12, at least about 13, at least about 14, at least about 15, at least about 16, or at least about 17 days following transfection, following administration to the subject, or both.

In certain embodiments of the method or methods described herein, the method may further comprise a step of administering a prodrug, such as 5FC, 5FU, ganciclovir, or any combination thereof, to the subject such that the MSC or MSCs are exposed to the prodrug, such as 5FC, 5FU, ganciclovir or combination thereof, and may convert the prodrug to active form.

In certain embodiments of any of the above method or methods, the method may further comprise a step of producing the transfected MSC or MSCs using any of the production method or methods as described herein prior to the step of administering the MSC or MSCs.

In certain embodiments, expansion and/or culturing of MSCs may be performed before transfection. By way of example, for large scale MSC modification such expansion may be desirable in certain embodiments.

### EXAMPLES

### Example 1: Non-Viral Modification of Mesenchymal Stem Cells for Cancer Therapy. Efficient Non-Viral Processes for Engineering Mesenchymal Stem Cells for Gene Directed Enzyme Prodrug Cancer Therapy

Modification of mesenchymal stem cells (MSCs) for prodrug gene therapy has been made by viral and non-viral gene delivery systems. Due to the poor efficiency of traditional transfection approaches (0-50%), viral methods have been used extensively in preclinical and clinical studies. Embodiments described herein demonstrate polyethylenimine (PEI) based modification of human adipose tissue derived MSCs (AT-MSCs) at >90% efficiency in the presence of one or more of a first agent, such as fusogenic lipids, and second agent, such as histone deacetylase 6 inhibitor (HDAC6i). The cell phenotypes of MSCs remained unchanged after modification, a desirable feature for clinical application of MSCs. Armed with this method, the anticancer efficacy of modified MSCs producing fused yeast cytosine deaminase::uracil phosphoribosyltransferase (CDy::UPRT) was examined in glioma, breast and gastric cancer cell lines. Through efficient conversion of 5-fluorocytosine, CDy::UPRT_AT-MSCs exhibited strong cytotoxic effect towards human gastric, breast and glioblastoma cancer in vitro. More than 80% inhibition was observed in gastric MKN1 cell line when directly cocultured with 1% of therapeutic CDy::UPRT_AT-MSC/5FC. Prolonged expression up to 7 days post transfection was possible with CpG free expression plasmid. CDy::UPRT_AT-MSCs collected 7 days post transfection showed efficient inhibition of 85% and 95% in gastric MKN1 and MKN28 cell lines, respectively. Results indicate that the presently described methods may offer an alternative process for MSC-based prodrug therapy without the use of viral vectors.

Cumulative evidence of the inherent tumour tropism of MSCs has opened up an emerging platform to utilize MSCs as cell vehicles to deliver anticancer agents specifically to tumors and their metastatic sites [9, 10]. Recently, a number of MSC-driven GDEPT clinical trials have presented promising results that warrant further developments into phase II trials [7, 11]. This therapeutic approach enables localized and controlled conversion of the non-toxic prodrug enzymatically in close proximity to the target cells. The ‘by-stander effect’ increases the cytotoxicity against target cells [7]. The anticancer potential of CD-producing MSCs has been validated in broad spectrum of solid cancers [7, 8], including gastric cancer [12-14], breast cancer [15, 16], and glioblastoma [17-19]. Preclinical studies have demonstrated that cytosine deaminase/5-fluocytosine (CD/5FC) is highly robust, where as low as 4% of CD positive cells in the tumour mass is sufficient to completely eradicate the tumour [20-22]. A significant advancement with the CD/5FC system was the inclusion of uracil phosphoribosyl-transferase (UPRT), a pyrimidine salvage enzyme that directly converts 5FU to 5-fluorouridine monophosphate (FUMP), thus bypassing the rate-limiting enzymes Dihydropyrimidine dehydrogenase (DPD) and Orotate phosphoribosyltransferase (OPRT) [23-26]. CD::UPRT/5FC enhances the conversion of 5FC into its active metabolites by 30-1500 folds in comparison to CD/5FC and 5FU [24, 27].

Transient transfection may have a high payload per cell, avoiding antibiotic selection and weeks of process work that may cause cell senescence [40] and reduce tumour tropism [41] as well as safety concerns with viral induced MSC transformation [42]. Although non-viral methods have advantages over viral vectors for the ease of production, low cost and safety profiles [43], the lack of wide adoption for MSC modification is mainly due to the low efficiency of transfection (0-35%) typically encountered [44, 45]. For instance, due to the poor performance of chemical based transfection methods (<5% efficiency) [46], human adipose tissue derived MSCs (AT-MSCs) have been engineered by retrovirus transduction to express CD::UPRT [47, 48].

In the present studies, it was possible to modify AT-MSCs and other MSC source at high efficiency using cationic polymer in combination with TrafEn, enabling the development of therapeutic MSCs producing CDy::UPRT without the need to use virus or establishment of stable cell line.

Results

An Efficient Non-Viral LPEI Based Transfection Method for AT-MSCs Modification

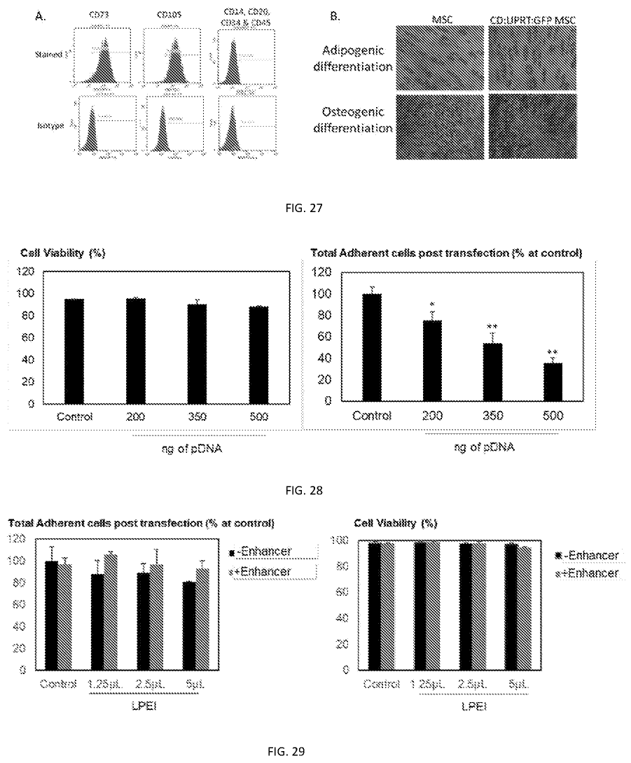

AT-MSCs (Age group 18-30) were transfected with a plasmid encoding GFP reporter gene in 24-well tissue culture vessels to evaluate the transfection efficiencies of LPEI and Lipofectamine 3000 (L3K). Although, there were more cells transfected using LPEI, the number of adherent cells were less than when using L3K (FIG. 8A). While the cell viability post-transfection remained high, there was a significant reduction in adherent cell number after LPEI mediated transfection when compare to un-transfected control. The number of adherent cells further reduced with the use of increasing amounts of pDNA (FIG. 28), consistent with previous observations (Swiech, et al., BMC Biotechnology, 11(114), 2011; McCall et al., Frontiers in Molecular Neuroscience, 5, (2012); Ho et al., Bioscience Reports, 38, 2018; Madeira, et al., Journal of Biotechnology, 151, 130-136, 2011). Attempts to attain high adherent cell number by transfecting AT-MSCs at 200 ng of pDNA with lower amounts of polymers only resulted in significantly reduced transfection efficiency (FIG. 8B).

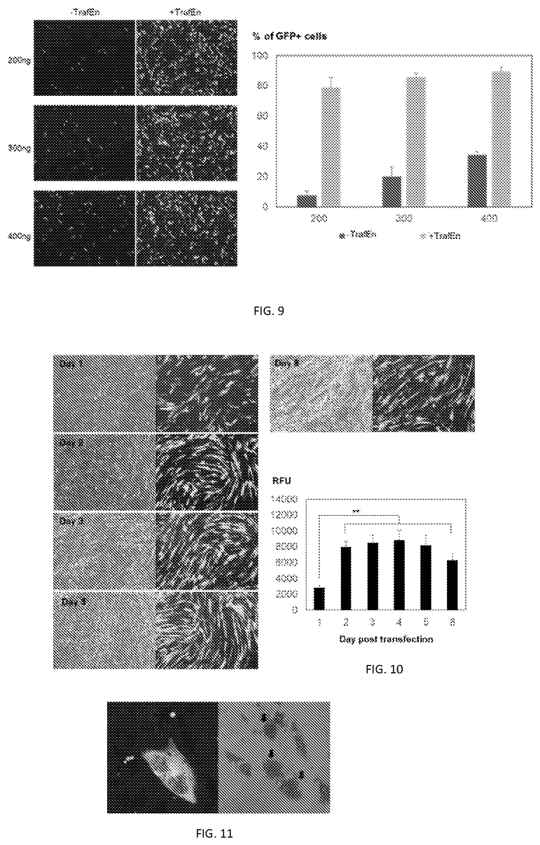

Next, we explored the use of the Enhancer 035 with low amount of pDNA (200 ng) and various ratios of DNA:polymer for the enhancement of transfection (FIGS. 8b and 29). More than 80% of AT-MSC cells were transfected (FIGS. 8B and 29), with comparable number of adherent cells and viability to non-transfected control (FIG. 29). We next extended the study to include other AT-MSC isolated from another donor (Age group 31-45). Using the same protocol, the transfection efficiency was as high as 90% of cells transfected (FIG. 9).

To establish a reliable protocol for gene modification of AT-MSCs, we tested and compared transfection of LPEI and Lipofectamine 3000 at various amounts of DNA. AT-MSCs (Age 18-30) were transfected with a plasmid encoding GFP reporter gene in 24-well tissue culture vessels to monitor the efficiencies of various parameters (FIG. 8A). Evidently, AT-MSCs were recalcitrant to Lipofectamine 3000 transfection. While LPEI outperformed Lipofectamine 3000 in transfection efficiency, significant reduction in cell number was observed, suggesting potential cell burdening.

However, attempts to transfect AT-MSCs at low amount of pDNA and polymers so as to reduce cell burdening, substantially reduced transfection efficiency (FIG. 8B). Our previous study in unravelling the intracellular trafficking pathways of polyplexes led to the development of TrafEn based method [54]. TrafEn may comprise histone deacetylate inhibitor (HDACi) and fusogenic lipids. In the studies herein, we examined the potential of these reagents in enabling efficient transfection at low amount of pDNA and polymers. More than 80% of AT-MSC cells were transfected without burdening of cells (FIG. 8B). We next extended the study to include other AT-MSC isolated from another donor (Age 31-45). Using the same protocol, close to 90% transfection efficiency was achieved (FIG. 9).

Human adipose tissue derived mesenchymal stem cells (AT-MSCs, RoosterBio) were isolated from female donor (LOT00088, age 18-30). AT-MSC was maintained in the hMSC High Performance Basal Media (Roosterbio). Breast cancer cell line MDA-MB-231 (HTB-26, ATCC), and primary human dermal fibroblast (ATCC, PCS-201-012), were cultured and maintained according to manufacturer's instruction. Glioma cell line U-251MG was kindly provided by Paula Lam (Duke NUS Medical School). U-251MG cell line was cultured in DMEM (Dulbecco Modified Eagle Medium) supplemented with 10% Fetal Bovine Serum (FBS, Biowest). Gastric cancer cell line MKN1 and MKN28 was kindly provided by Dr. Yong Wei Peng (National University Cancer Institute, Singapore). The gastric cancer cell lines were cultured in RPMI (Roswell Park Memorial Institute medium, Thermo Scientific), supplemented with 10% FBS. Cells were kept at 37.0 in humidified atmosphere and 5% CO2.

Characterization of Theranostic CDy::UPRT_AT-MSCs, and Determination of the Functionality of CDy::UPRT_AT-MSCs

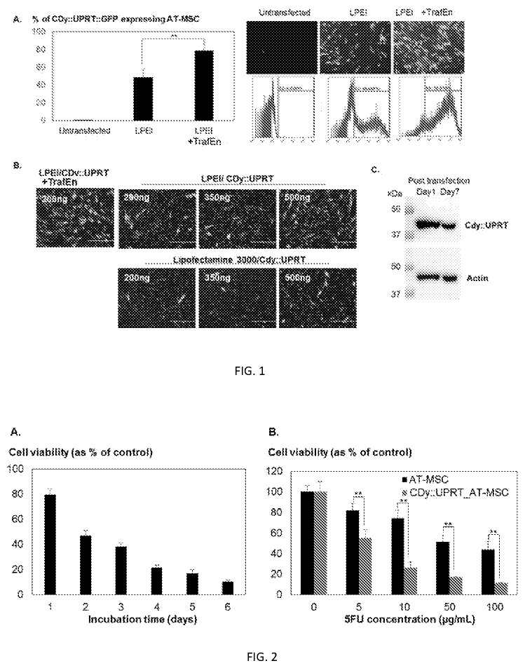

To generate AT-MSCs expressing fused cytosine deaminase and uracil phosphoribosyltransferase (CDy::UPRT_AT-MSCs), AT-MSCs were transfected with LPEI following the centrifugation protocol. In the presence of TrafEn, transfection efficiency close to 80±2.3% was reachable, based on the GFP analysis of AT-MSCs transfected with CDy::UPRT::GFP (FIG. 1A). As shown in the Flow Cytometry analysis, majority of the transfected cells expressed high level of GFP in the presence of TrafEn. While increasing DNA amount improve transfection efficiency of LPEI and Lipofectamine 3000 moderately, their performance remains unsatisfactory (FIG. 1B). These data suggest TrafEn are required to facilitate intracellular trafficking of polyplexes in the AT-MSCs [49]. Duration of CDy::UPRT expression was confirmed to be maintained for at least 7 day post transfection (FIG. 1C, 10).

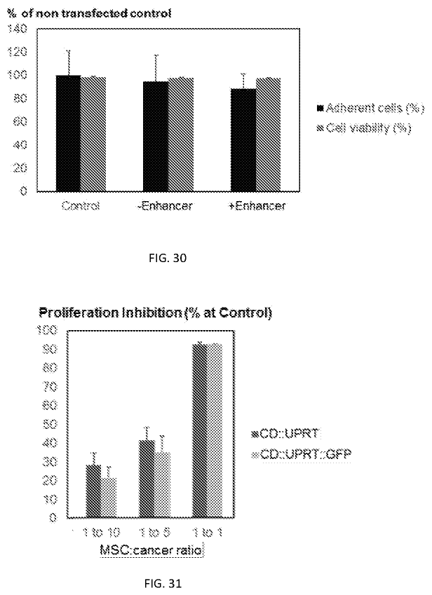

Based on immunocytochemistry analysis, transfection was significantly improved in the presence of Enhancer at low amount of pDNA (200 ng). In the absence of the Enhancer, increasing pDNA amount modestly increased transfection efficiency of LPEI and Lipofectamine 3000 (FIG. 1). Extending this observation, we constructed a fusion gene encoding cytosine deaminase, uracil phosphoribosyltransferase and green fluorescent protein (CDy::UPRT:GFP) for direct visualization and quantification. In the presence of Enhancer, transfection efficiency was significantly increased (˜80%) as compared to the use of LPEI alone (˜40%; FIG. 1), with no significant change in viability (FIG. 30). Notably, there was no significant difference in the anti-cancer efficiency of AT-MSC modified with CDy::UPRT:GFP or CDy::UPRT (FIG. 31). Collectively, the results demonstrated a significant improvement in the transfection of AT-MSCs by the Enhancer, which likely shares a similar mechanism in facilitating intracellular trafficking of pDNA in BM-MSC (Ho et al., Nucleic Acids Research, 45(38), 2017).

Transgene of interest was introduced into AT-MSCs at passage 3-5. For each well (6-well plate format), 5 mg/mL of LPEI (PEI MAX, Polyscience) was added to pDNA in serum free DMEM at different ratio of pDNA and PEIMAX. The mixture, at a total volume of 1000, was incubated at room temperature for 15 min. The pDNA:LPEI ratio was calculated according to the amount of pDNA, μg: volume of 1 mg/mL of LPEI, μl. LPEI/pDNA complex was then added to serum free DMEM medium (1:20) to prepare the transfection mixture. The culture media was removed and replaced with the transfection mixture, followed by mild centrifugation at 200 g for 5 min. After centrifugation, the transfection mixture was removed and replaced with complete media, with or without supplementation of TrafEn. TrafEn consist of DOPE/CHEMS (Polar Avanti Lipid) and Vorinostat (SAHA, Bio Vision). Cells were incubated for 24 h before analysis.

Flow cytometry, western blot and immunocytochemistry were performed as previously described [49]. Briefly, Flow cytometry: Percentage of fluorescence positive cells was quantified by Attune NxT Flow Cytometer system (ThermoFisher Scientific) and the raw data was analysed using Invitrogen Attune NxT software (ThermoFisher Scientific). Imaging: Cell images were taken with EVOS FL Cell Imaging System (ThermoFisher Scientific) equipped with three fluorescent light cube for viewing of DAPI (Ex357/Em447), GFP (Ex470/Em510) fluorescence. Western blot: Samples were analysed by immunoblotting technique with sheep anti-CDy (PA185365, ThermoFisher Scientific) and monoclonal anti-β-Actin (A2228, Sigma-aldrich), respectively. Immunocytochemistry: The samples were labelled with sheep anti-CDy and Alexa Fluor 488 donkey anti-sheep fluorescent secondary antibody (A11015, ThermoFisher Scientific). Image acquisition was performed using the EVOS FL Cell Imaging System. All images were taken with identical optical settings.

Expression of CDy::UPRT rendered AT-MSCs sensitive to prodrug 5FC (exposure of CDy::UPRT modified AT-MSCs to 5FC reduced cell viability over time) (FIG. 2A), demonstrating the functionality of the phosphoribosyltransferase domain of the CDy transgene. Furthermore, CDy::UPRT_AT-MSCs demonstrated increased susceptibility of suicide effect in the presence of active cytotoxic drug 5FU (FIG. 2B). This effect was likely to be due to the activity of UPRT transgene, which catalyzes the conversion of 5-FU to 5-fluorouridine monophosphate [25]. This is in line with the observations in other studies [19, 55].

Phenotypic Characteristics of AT-MSC are not Affected by the LPEI Based Transfection Method.

For future clinical use, it is desirable to ensure the quality of AT-MSCs remained unchanged after genetic modification. To exclude the possibility, that high transfection efficiency in AT-MSC could have been achieved at the expense of AT-MSC quality, the characteristics were assessed according the main criteria defined by International Society for Cellular Therapy (ISCT)[51]. Indeed, to explore the possibility that high transfection may modify the phenotype of AT-MSC, immunophenotyping of CDy::UPRT_AT-MSCs was carried out by standard FACS analysis using markers as defined by the International Society for Cellular Therapy (ISCT) [51]. With unmodified AT-MSCs as reference, we analysed the immunophenotype of CDy::UPRT_AT-MSCs by standard FACS analysis for potential change in the expression of surface markers. The CDy::UPRT_AT-MSCs displayed identical profile in comparison to the unmodified AT-MSCs. Both cell types were found to be positive for CD90, CD73 and CD105 while negative for CD14, CD20, CD34, CD45 and HLA-DR (FIG. 3A, 27B). Expression of CDy::UPRT did not affect the differentiation capability of AT-MSCs into osteogenic (FIG. 3B, 27A) and adipogenic lineages (FIG. 3C, 27A). Evidently, the presence of oil droplets in the CDy::UPRT::GFP expressing AT-MSCs provides direct evidence for the differentiation potential of AT-MSC post transfection (FIG. 11). Oil droplets indicated the potential to differentiate into adipogenic lineage was unaffected by transfection and transgene expression (FIG. 11). In a separate study, chondrogenic differentiation was also unaffected after transfection using this method (not shown).

To examine the phenotype of CDy::UPRT producing AT-MSCs, cells were labelled with MSC Phenotyping Kit consisting of antibodies CD73, CD90, CD105, CD14, CD20, CD34, CD45, and HLA-DR (Miltenyi Biotech) according to manufacturer's instructions. After which, expression of the markers were analysed with FACS. High quality MSC population consist of >95% CD90, CD105, and CD73 positive cells. The population expressing CD14, CD20, CD34, CD45, and HLA-DR may be less than 1% [51]. The multipotency of AT-MSCs was confirmed by its differentiation capacity into osteogenic and adipogenic lineage [52, 53]. Differentiation of AT-MSCs was induced with StemPro™ Osteogenesis Differentiation Kit and StemPro™ adipogenesis Differentiation Kit (ThermoFisher Scientific). Unmodified AT-MSCs were used as control. The phenotype and differentiation potential of CDy::UPRT producing AT-MSCs may not vary significantly from the unmodified AT-MSC.

CDy::UPRT_AT-MSCs Retain Tropism for Cancer Cell Lines In Vitro

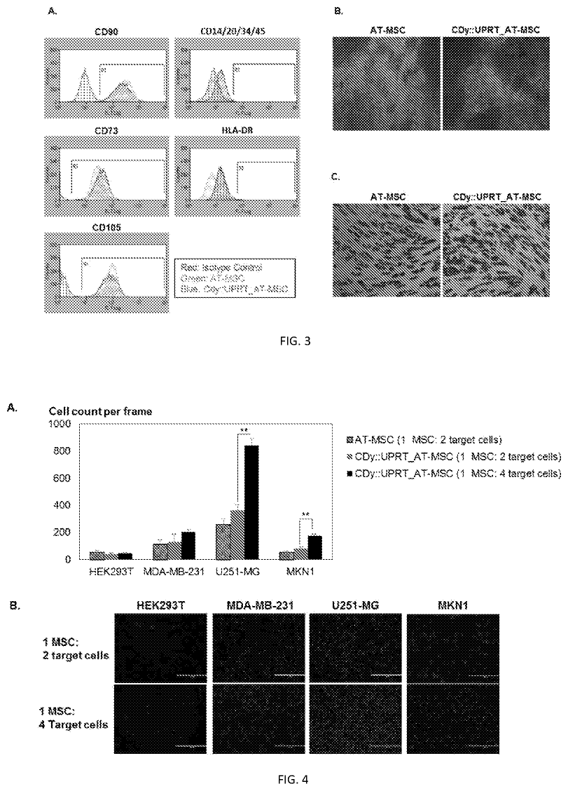

The chemotactic response of AT-MSCs toward the cytokines released by cancer cells is desirable for successful targeting of tumor cells [10]. Thus, genetic modification desirably does not alter tropism of AT-MSCs for cancer cells. Here, invasion assay was used to examine the potential impact of LPEI based transfection on the tumour tropism of AT-MSCs. Vectorial migration of AT-MSCs through extracellular matrix in the presence of cancer cells was investigated. Directionally migration of AT-MSCs through extracellular matrix demonstrates tropism of AT-MSCs for cancer cells. Invasion of AT-MSCs through extracellular matrix was significantly induced by MDA-231-MB, U251-MG and MKN1 but not HEK293T (FIG. 4A). This observation is in line with other studies as HEK293T served as non-cancerous cell line control [56, 57]. Comparable number of migrated AT-MSCs and CDy::UPRT_AT-MSCs suggests tumour homing capability was not affected by LPEI mediated transfection and CDy::UPRT expression in AT-MSCs. To further confirm migration of AT-MSC is dependent on the specific chemokines secreted by cancer cells, we hypothesized that higher number of cancer cells should result in increasing migratory AT-MSCs. Indeed, significant higher number of CDy::UPRT_AT-MSCs migrated towards U-251MG and MKN1. On the other hand, moderate increment of migrated AT-MSCs was found in conditions with MDA-MB-231 cell line (FIG. 4). The number of CDy::UPRT_AT-MSCs invaded through the extracellular matrix was dependent on the number of cancer cells, with higher numbers of cells migrated towards U-251MG and MKN1, and lesser towards MDA-MB-231 cell lines (FIG. 4).

Quadruplicates of AT-MSC, MKN1, MKN45, MDA-MB-231 (10,000 cells per well) and U-251MG (4000 cells per well) for each treatment were plated into 96-well plates. Twenty four hours later, culture medium was replaced for medium containing various concentration of 5-Fluorocytosine (5-FC, InvivoGen) or 5-Fluorouracil (5FC, InvivoGen). One to five days later, plates were subjected to the CellTiter 96 Aqueous One Solution Cell Proliferation Assay (Promega). The colorimetric read out was measured spectrophotometrically at 490 nm. Results were expressed as the percentage of cell viability, in relative to cells in condition without 5-FC or 5-FU (set to 100%).

An exemplary method of the cell invasion assay is as follows. The tumour tropism of AT-MSCs was determined using BD Biocoat™ matrigel invasion chambers (BD Biosciences). Cancer cell lines or HEK293T cells were loaded in the lower well of the 24-well plates. Twenty four hours later, unmodified and CDy::UPRT-producing AT-MSCs in serum-free DMEM were added onto the invasion chambers. Lower wells were washed with 1×PBS, filled with serum free DMEM, assembled for the invasion assay. After 24 h incubation, non-invading cells and matrigel were removed from the inside of the insert. Invaded cells were stained with Hoechst 33342 (ThermoFisher Scientific) and photographed through the imaging system. Number of cells in 3 frames were counted.

CDy::UPRT_AT-MSC/5FC Mediated Cytotoxicity In Vitro

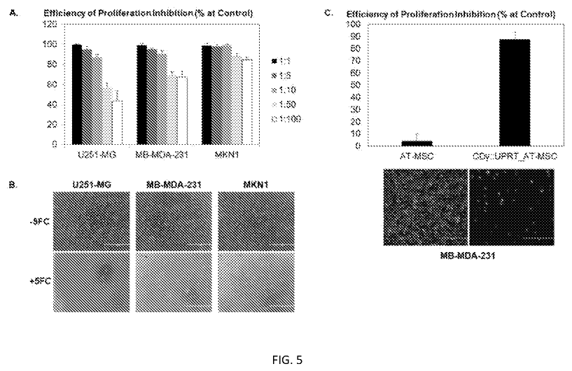

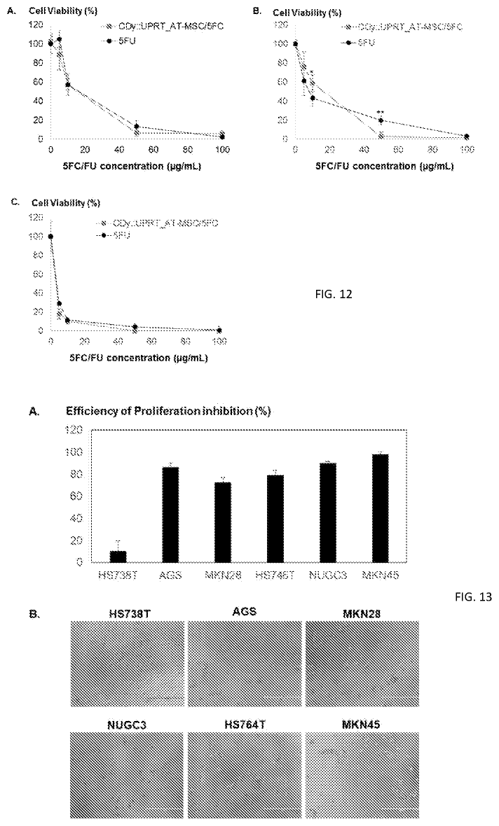

Demonstration of cytotoxic effect of the CDy::UPRT_AT-MSCs on target cells is key for adoption of LPEI based transfection/TrafEn in the generation of theranostic MSCs for prodrug cancer therapy. The effect of cytosine deaminase/5FC in proliferation inhibition is commonly assessed by MTS assay. We first compared the anti-cancer efficiency of CDy::UPRT_AT-MSC/5FC and 5FU in glioma, breast cancer and gastric cancer cell line (FIG. 12). Comparable anticancer effect of CDy::UPRT_AT-MSC/5FC and 5FU suggest high efficiency in converting 5FC to cytotoxic drug. At 1:1 ratio of CDy::UPRT_AT-MSC to cancer cells, the anti-cancer effects were comparable to the direct pharmacological effects of 5FU. To further examine the therapeutic potential of CDy::UPRT_AT-MSC/5FC, cells were directly cocultured with target cancer cells at various MSC to cancer cell ratios (FIG. 5A). Proliferation inhibition by almost 57%, 69% and 89% could be observed even at coculture ratio of 1:50 of CDy::UPRT_AT-MSC/5FC to U251-MG, MDA-MB-231, and MKN1, respectively. This ratio of mixed culture represents 2% of therapeutic cells within the cancer cells. More than 86% proliferation inhibition could have been attained in all cancer cells when 10% of therapeutic cells were used. It is worthy to note that 85% proliferation inhibition was seen with only 1% of therapeutic cells in the MKN1 population. Proliferation inhibition was not observed in cocultures without 5FC, suggesting the lack of anti-cancer properties of AT-MSCs (FIG. 5B).

An exemplary method of direct coculture methodology is as follows. Quadruplicates of gastric cancer cell lines and breast cancer cell line (5000 cells) and U-251MG (2000 cells) were plated in 96-well plates. Five hours later, increasing numbers of either unmodified or CDy::UPRT-producing AT-MSCs at the ratios of 1 AT-MSC to 1, 5, 10, 50 and 100 cancer cells were added to the cancer cell culture. One day later, the culture media was replaced with DMEM supplemented with 2% FBS, with or without 5-FC (0-150 μg/mL). Five days later, cell viability was measured by proliferation assay. Conditions without 5-FC was set to 100%.

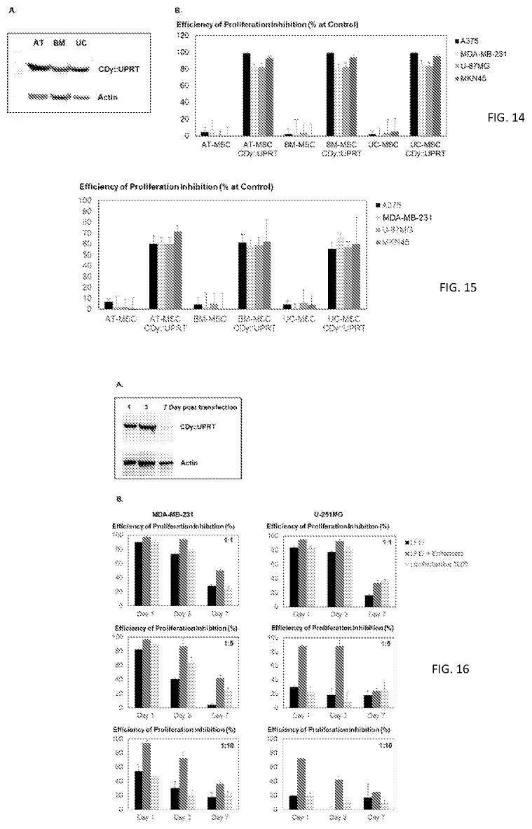

In view of the situation where the therapeutics cells might not be in direct contact with the cancer cells in vivo, indirect coculture experiment was used to access the cytotoxic effect of CDy::UPRT_AT-MSC/5FC. Four day after exposure of MDA-MB-231 to CDy::UPRT_AT-MSC/5FC, close to 90% proliferation inhibition was observed (FIG. 5C). The anticancer efficiency of CDy::UPRT_AT-MSC/5FC in the absence of cell-cell contact is highly comparable to the direct coculture model. Taken together, these data suggest that potent cytotoxic anticancer effect may be exerted when therapeutic cells are in contact or close proximity to the target cells. We next extended the study to compare the sensitivity of normal mixed stomach cells (Hs738—non-transformed human fetal gastric/intestinal cells) to 5 gastric cancer cell lines. CDy::UPRT_AT-MSC/5FC exerted cytotoxic anticancer effect selectively to gastric cancer cell lines (FIG. 13), suggesting specific targeting of the therapeutic cells/5FC to cancerous but not normal cells.

An exemplary method of indirect coculture methodology is as follows. MB-MDA-231 cells were plated on 24-well plate (5×10⁴ cells per well). AT-MSCs or CDy::UPRT_AT-MSCs (5×10⁴ cells per well) were plated on transwell (Corning, C05/3422). After 6 h of cultivation, inserts with therapeutic cells were transferred into the wells with MB-MDA-231 cell line, with or without 5FC. Cytotoxic effect was evaluation after 4 days of incubation. Transwells were removed and culture media was replaced with 1×PBS containing 1 μg/mL of Hoechst 3222. Stained cells were analysed using Synergy H1 microplate reader at excitation and emission wavelength of 358 nm and 461 nm, respectively. With gain setting at 80, RFU at 9 areas of the cell culture were recorded.

LPEI/TrafEn Enhancer Generates Highly Potent CDy::UPRT_AT-MSCs

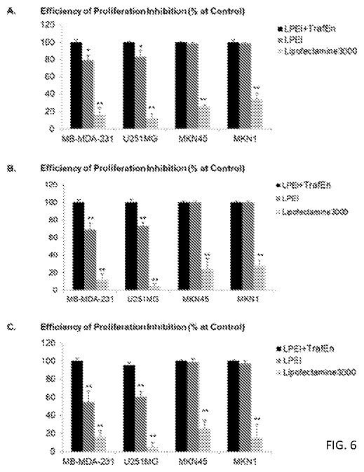

We hypothesized that high expression of suicide gene may be important in the process of generating therapeutic AT-MSCs. Next, we compared the potencies of the therapeutic cells processed with different protocols that have been tested by transfecting AT-MSCs with pCMV-GFP (FIG. 8) or CpG free plasmid encoding for CDy::UPRT (FIG. 1). As expected, the anticancer efficiencies of the therapeutic cells prepared with different protocols were highly dependent on transfection efficiencies of each protocol (FIG. 6). The anticancer efficiency of CDy::UPRT_AT-MSCs generated in the presence of TrafEn (enhancer) significantly surpass the other methods, especially in MB-MDA-231 and U251-MG. These two cell lines demonstrated poorer susceptibility to 5FU toxicity (FIG. 12) and higher concentration of 5FU was required to inhibit their proliferation. At the ratio of 1 MSC to 10 cancer cells, complete inhibition of proliferation was observed in all cancer cell lines cocultured with CDy::UPRT_AT-MSCs generated in the presence of TrafEn. It is worthy to note that the current method described here is applicable to MSCs obtained from various sources (FIG. 14) and other suicide gene such as Herpes Simplex Virus-1 Thymidine Kinase (HSV-TK) (FIG. 15). Transfection protocol using the Enhancer generated modified MSCs with similar potencies regardless of cell sources (adipocyte, bone marrow or umbilical cord derived MSCs; FIG. 14). Furthermore, we have successfully transfected MSCs with another suicide gene such as Herpes Simplex Virus-1 Thymidine Kinase (HSV-TK) (FIG. 15).

Long Term Expression of CDy::UPRT in AT-MSCs with Transient Transfection

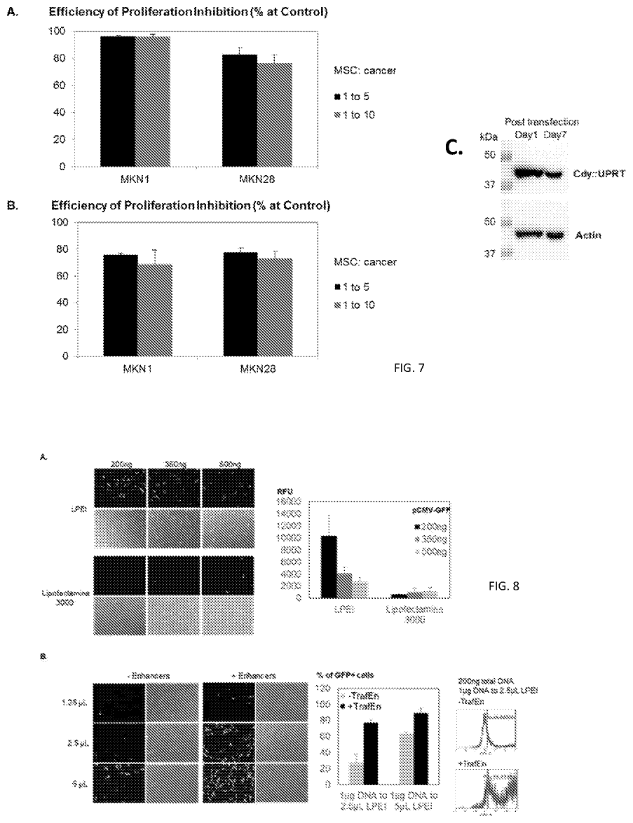

Based on the evidences on biodistribution of MSCs in vivo, it is anticipated that 1 to 4 days are taken for MSCs to spread out among residual tumour and home to distant foci of tumour, depending on the route of administration and location of the tumour [29, 58, 59]. To ensure continued expression of the suicide gene, viral transduction and subsequent antibiotic selection were adopted in the preparation of modified MSCs for prodrug therapy [19, 29, 60]. We have demonstrated that continued expression of CDy::UPRT up to 7 day post transfection (FIG. 1C). To verify that the modified AT-MSCs remain functional within the duration suitable for substantial anti-cancer effect, the cancer killing efficiency of modified MSCs collected from day 1 and day 7 post transfection were determined in a parallel study. Comparable proliferation inhibition was attained with CDy::UPRT_AT-MSCs harvested on day 1 and day 7 post transfection (FIG. 7).

CpG free plasmid was deliberately selected in this study as enhanced and prolonged expression was observed with plasmid without CpG [61, 62]. Also, the plasmid backbone used in this study contains matrix attachment region (MAR) to improve the stability of gene expression [63]. Indeed, with expression plasmid without these two features, expression of CDy::UPRT reduced drastically at day 3 post transfection. As expected, the anticancer efficiency of the modified AT-MSCs collected on day 7 post transfection was 400 times poorer than its counterpart collected on day 1 post transfection (FIG. 16). While AT-MSCs modified in the presence of TrafEn demonstrated strong anticancer potency in comparison to other methods, the efficiency of proliferation inhibition decreased over time, suggesting the bottleneck lies in the events after successful gene delivery. It is worthy to note that TrafEn enabled superior anti-cancer efficacy despite the reduction of the functionality of modified AT-MSC over time.

Indeed, prolonged expression of CDy::UPRT in AT-MSCs was possible with transient transfection. To investigate the duration of expression and function of the transgene in modified AT-MSCs, the anticancer efficiency of modified MSCs collected from day 1 and day 7 post transfection were examined. Evidently, the expression of the transgene, CDy::UPRT, was significant over a period 7-day post transfection (FIG. 7C), consistent with the observation using CD::UPRT::GFP (FIG. 10). Comparable proliferation inhibition of cancer cells were observed with CDy::UPRT_AT-MSCs harvested on day 1 (FIG. 7A) or day 7 post transfection (FIG. 7B).

Administering MSC

The manner of MSC administration may depend on the type of cancer and/or the particular application. The upstream process development to generate therapeutic MSC for suicide prodrug therapy is described herein. Modified MSC may be administered, for example, intravenously for adenocarcinoma treatment (as in TREAT-ME phase 1 trial, Oncotarget. 2017 Oct. 6; 8(46): 80156-80166). In certain embodiments, intracranial administration may be performed, for example as in recurrent high grade glioma patients underwent intracranial administration of CD-NSCs during tumor resection or biopsy (Clin Cancer Res. 2017 Jun. 15; 23(12):2951-2960).

Prodrug/Gene Combinations.

The present studies further relate to fused genes of a cytosine deaminase/uracil phosphoribosyltransferase and 5-flucytosine system. We have also tested thymidine kinase/ganciclovir system. Similarly, TrafEn method outperformed other non-viral methods in anti-cancer efficiency (FIG. 14B). The presently described methods may serve as a platform for non-viral modification of various MSC types for suicide gene/prodrug systems known in the art, such as those listed in Table 3 in J Clin Invest. 2000 May 1; 105(9): 1161-1167 which is incorporated herein by reference in its entirety.

Comparing Non-Viral to Viral Transfection

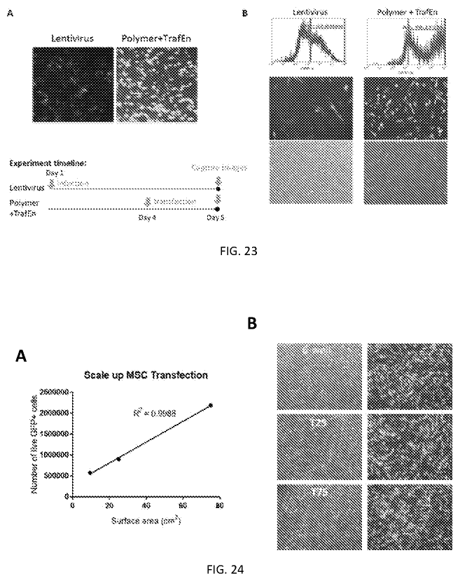

TrafEn, a formulation of reagents (as described in PCT/SG2013/000464), enables productive expression of transgenes with the high copies of intracellular plasmid DNA in the studies described herein, resulting in the generation of MSC with high therapeutic payload in the present studies. A significant advantage of TrafEn system over viral method is the early onset of transgene expression (FIG. 23A) and the significantly higher expression per cell (FIG. 23B). These features may enable the shortening of cell preparation process and higher payload of suicide gene per cell, potentially reducing the production cost and the number of MSCs to be used for the treatment.

Generation of Stable Cell Line.

The generation of stable cell lines seeks to obtain high numbers of transfected cells. Antibiotic selection is highly labor intensive (2-3 weeks) and may potentially compromise MSC quality [88], cause cell senescence [88] and reduce tumour tropism [89] as well as safety concerns with viral induced MSC transformation [90]. Without antibiotic selection, poor transfection efficiency may not be sufficient for clinical application. In view of the successful high transfection using the processes developed and described herein, there is no longer the need to generate stable cell lines. With the therapeutic gene subcloned into a cpg free vector, the high expression of CDUPRT over a 7 day period was as effective in eliminating cancer cells as those 1 day after transfection (FIG. 7A, 7B). While it is contemplated that selection is compatible with the transfection methods described herein, the transfected MSCs here were not selected as the efficiency was >80%. In certain embodiments, it is contemplated that generating a stable cell line is unnecessary for the therapy.

CD::UPRT_AT-MSC Mediated Tumor Growth Inhibition In Vivo

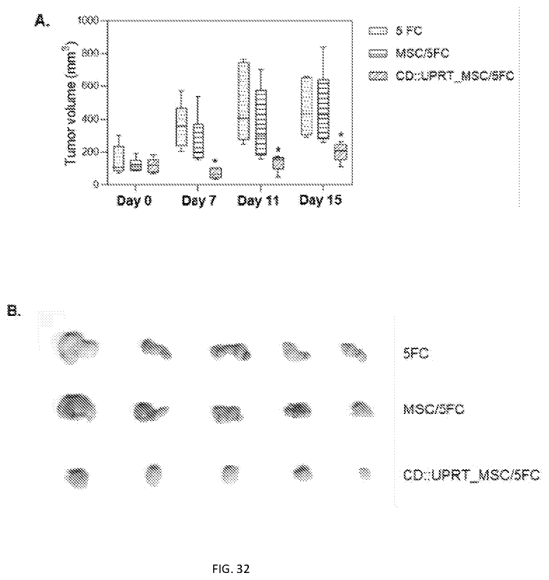

To examine the approach in vivo, CD::UPRT_AT-MSCs were injected directly into the subcutaneous (s.c.) tumour. Sizeable studies have come up with rather contradictory outcomes regarding the use of non modified MSC for experimental cancer treatment (Christodoulou et al., Stem Cell Res Ther, 9(336), 2018). To ensure the anti-tumour effect was due to the expression of CD::UPRT, cell control group (MSC plus 5FC) was used in addition to the prodrug control group (5FC). In the current study, MSC plus 5FC did not exert significant pro- or anti-tumour effect. Significant inhibition of tumor growth was observed in the treatment group (FIG. 32). With one cycle of treatment, an average of 45% reduction in tumour size was observed in the treatment group 3 days after the last 5FC administration (Day 7 in FIG. 32A). The overall tumour size in the treatment group is significantly smaller than the prodrug and cell control group (FIG. 32B).

These results provide in vivo evidence of CDEPT efficacy in subcutaneous mice model-glioblastoma cell line U251MG.

Discussion

The therapeutic value of GDEPT mediated by MSCs has been proven in various preclinical [9, 20, 21] and clinical trials [7, 9]. Viruses are used as means for genetic modification of MSCs due to the lack of efficacy in alternative, non-viral based methods. The present studies demonstrate the use of a formulation and protocol for the generation of theranostic AT-MSCs with high pay-load for prodrug cancer therapy, without the need of virus. The beneficial outcome of an efficient transfection method was established in the substantial functional improvements in the anti-cancer potency of CDy::UPRT producing AT-MSCs. The method described herein does not alter the quality and characteristics of AT-MSCs and is applicable to various types of MSCs and therapeutic genes.

Gene delivery method plays a profound role in the developmental process of MSCs driven prodrug gene therapy. Viral vectors are routinely used in the preclinical studies and clinical trials [28-31] to modify MSCs for GDEPT. Retrovirus is frequently used in the generation of CDy expressing AT-MSCs [19, 47, 48, 60, 64]. Briefly, AT-MSCs were transduced thrice in three consecutive days with retrovirus-containing medium. The cells are further expanded in the presence of antibiotic G418 for 10 days before cells before use. An alternative method is lentiviral gene delivery system. Up to 80% efficiency has been reported with single transduction in AT-MSCs at MOI of 150 [65, 66]. Unfortunately, polybrene could potentially inhibit MSC proliferation [67].

In comparison to viral gene delivery systems, we report a facile PEI based transfection method that is rapid, simple, cost-effective, without the need for antibiotics selection or multiple transfections. As each of the cell could be transfected with thousands of DNA copy [35, 36], majority of cells were found to express high level of the gene of interest (FIG. 1, 8B). This could potentially explain the high potency of CDy::UPRT_AT-MSCs. Indeed, CDy::UPRT_AT-MSCs generated by PEI+Enhancer demonstrated comparable anticancer efficiency to 5FU, suggesting efficient conversion of prodrug 5FC to toxic agent (FIG. 13). Without further purification or antibiotic selection post transfection, complete inhibition of MD-MBA-231, U251-MG, MKN45 and MKN1 cell lines was achievable with 10% of therapeutic AT-MSCs, generated by PEI based transfection in the presence of the TrafEn (FIG. 6C). On the other hand, the poor transfection efficiencies of PEI alone and Lipofectamine3000 (FIG. 1) in AT-MSCs resulted in the lower anti-cancer efficiencies, especially in MD-MBA-231 and U251-MG cell lines (FIG. 6).

The phenotypes and characteristics of the therapeutic AT-MSCs prepared with the method reported herein compare well with data presented in other studies, where AT-MSCs were modified with retrovirus to express CDy or CDy::UPRT for prodrug cancer therapy [19, 47, 48, 60, 64]. We assessed the phenotypic markers, differentiation potential and tumour tropism of CD::UPRT_AT-MSCs with reference to the non-modified AT-MSCs, confirming that our method does not affect the quality of AT-MSCs (FIG. 3, 4). This is a desirable feature for theranostic application of the modified AT-MSCs [51]. Consistent with other reports [60, 68, 69], CDy::UPRT_AT-MSCs were able to exert cytotoxicity in indirect co-culture experiments (FIG. 5C), further confirming that cell-to-cell contact is not required for the bystander effect. Similarly, it is noteworthy that the therapeutic MSCs is sensitive to the CDy::UPRT/5FC system (FIG. 2), thus preventing prolonged survival of the therapeutic cells; fulfilling the desired requirements as cell vehicles [69]. At the ratio of 1 therapeutic MSC to 10 cancer cell, Kucerova et al. demonstrated significant 40% proliferation inhibition MDA-MB-231 in the presence of CDy_AT-MSCs, generated with retrovirus transduction [47]. Interestingly, modification of AT-MSCs with CDy::UPRT does not improve the anti-cancer efficacy [16], leading to the effort on combinatorial prodrug treatment of CDy::UPRT/5FC and HSV-TK/GCV. Surprisingly, we observed inhibition of MDA-MB-231 proliferation at close to 91% at 10% of the therapeutic cells (FIG. 5A), possibly due to the higher expression level of the suicide gene per cell.

In a clinical trial of CD expressing neural stem cells, a treatment regime of 11 days were given to the recurrent glioma patients [70]. In the in vivo animal studies, the duration of treatment ranges from 6 [28, 29, 47] to 23 days [29], depending on the cancer types. Often, modified MSCs were given every week over a duration of 3 weeks. In this study, we successfully demonstrated prolonged expression of CDy::UPRT up to 7 day in AT-MSCs transfected with PEI plus TrafEn, without antibiotic selection (FIG. 1, 7). The expression of suicide gene in the transiently transfected AT-MSCs was sustainable throughout the required duration of the treatment regime [70]. This warrants the adoption of non-viral gene delivery system in the development of stem cell driven prodrug therapy. Evidently, AT-MSCs modified with CD::UPRT::GFP displayed comparable anti-cancer efficiency as CD::UPRT (Data not shown). Instead of antibiotic selection that could potentially affect MSC quality [40], GFP tag may be exploited for Flow cytometry isolation of CD::UPRT positive AT-MSCs, further streamline the workflow for theranostic cell generation.

The accelerating number of gene and cell therapy clinical trials indicates a thrilling era that promises an emergence of this therapeutic paradigm for diseases which previously lack treatment options. This trend has led to a greater demand for clinical grade virus manufacturing capacity. The shortage of virus production has been a bottleneck for advancement and commercialization of cell and gene therapy [71]. This study describes a useful tool for polymer based ex vivo MSC modification which may be highly scalable and cost-effective. The proposed workflow bypasses the restriction in viral vector supply and expedites MSC modification, without compromising the quality and anti-cancer efficacy of the theranostic MSCs. By having efficient non-viral based gene modification workflow, it has not escaped our notice that this method may be useful in achieving further therapeutic effect through co-transfection of multiple anticancer genes at ease, which may significantly broaden the horizon of therapeutic strategies for cancer treatment.

Conflicting reports have recently emerged regarding the roles of MSCs in tumor inhibition and growth. These contradictions were thought to be largely due to technical differences and inherent biological heterogeneity. Regardless, it is contemplated that genetically modified MSCs may offer a more suitable strategy for cancer therapy, as they are typically safer and more efficient than the unstable and heterogeneous naive MSCs.

This study achieved the successful modification of AT-MSCs at high efficiency for the generation of theranostic AT-MSCs for prodrug cancer therapy, without the use of viruses. About half of the cell population was transfected with the commercially available polymer (PEI-max) and the efficiency was significantly improved with low toxicity in the presence of the Enhancer (FIG. 1a). This modification process did not require purification nor antibiotic selection for high expression MSCs of >70% CD expressing cells, in line with release testing for human clinical trial. Attempts to develop novel cationic polymers and lipids to modify MSCs have previously been met with limited success due to low efficiency of transfection or high cytotoxicity. Recently, a poly(β-amino-esters) (PBAE) polymer structure was reported to transfect MSCs with high efficiency and low toxicity. Although the cells were well modified, the migration ability was notably affected.

In order for AT-MSCs to be used as targeted drug delivery vehicles for therapy, the processes used to modify them desirably does not significantly change their phenotypic characteristics and behavior, including their multipotency and their capacity for migration and invasion. No significant difference in the expression of phenotypic markers and differentiation potential of modified and native AT-MSCs (FIG. 3), a key criterion for theranostic application of the modified AT-MSCs. The inherent tumor tropism is a key feature of the homing/migration property of MSCs as a cellular vehicle for delivery of therapeutic agents. Despite the high over expression of the transgene, the migration ability of the modified cells was comparable to the native MSCs in the presence of cancer cells in vitro (FIG. 4).

A number of GDEPT systems is being explored for cancer treatment to improve the efficacy and safety of conventional cancer chemotherapies. Among the enzyme/prodrug systems tested in a recent study, the CD::UPRT is the most effective and this has been used with stem cells in clinical trials. In the present study, CD::UPRT modified cells inhibited the growth of MD-MBA-231, U251-MG, MKN45 and MKN1 cell lines efficiently, with as little as 10% of therapeutic AT-MSCs. Notable was that MDA-MB-231 proliferation was inhibited by ˜90% at the ratio of 1 therapeutic MSC to 10 cancer cell (FIG. 5A). At the similar ratio, Kucerova et al. demonstrated only 40% proliferation inhibition of the same cell type when using AT-MSCs modified by retroviral transduction (Kucerova, et al., J Gene Med, 10, 1071-1082, 2008). Yet another study reported ˜60% reduction in cell number in the co-culture of MDA-MB-231 with virally transduced CDy::UPRT MSCs at a ratio of 1 MSC to 4 cancer cells (Kucerova, et al., Stem Cell Research, 8, 247-258, 2012). Intratumoral administration of CD::UPRT_AT-MSCs/5FC showed that MSC modified with non-viral method is capable to exert higher anti-cancer effect (FIG. 32). With a comparable study design, Kwon et al. (Kwon, et al., Clinical and Experimental Otorhinolaryngology, 6, 176-183, 2013) and Nouri et al. (Nouri et al., Journal of Controlled Release: Official Journal of the Controlled Release Society, 200, 179-187, 2015) reported inhibition of tumour growth but not regression with 1 and 6 doses of CD MSC/5FC treatment cycle, respectively. Without wishing to be bound by theory, it is contemplated that the modification using the process developed herein may have resulted in increased payload resulting in more efficacious killing of cancer cells.

MSC mediated CD/5FC treatment has been suggested as a strategy to overcome the systemic toxicity of 5-FU (You, et al., Journal of Gastroenterology and Hepatology, 24, 1393-1400, 2009; Kwon et al., Clinical and Experimental Otorhinolaryngology, 6, 176-183). Throughout the in vivo study herein, we did not observe significant change in the weight of subjects or other direct side-effects (data not shown), as has been shown in other studies (You, et al., Journal of Gastroenterology and Hepatology, 24, 1393-1400, 2009; Kwon et al., Clinical and Experimental Otorhinolaryngology, 6, 176-183). Because of the alleviation of systemic toxicity, repeated injection of CD-MSC may be possible to enhance the antitumor activity. Additionally, it is noteworthy that the therapeutic MSCs is sensitive to the CDy::UPRT/5FC system (FIG. 2), thus limiting the survival of the therapeutic cells; fulfilling a key requirement of a ‘hit and run’ strategy, leaving no trace of the cell vehicle (Mohr, et al., Cancer Letters, 414, 239-249, 2018).

Depending on the route of administration and location of the tumour, it is contemplated that 1 to 4 days are appropriate for MSCs to biodistribute to residual tumour and home to distant foci of tumour (Aboody, et al., Sci Transl Med, 5, 184ra159, 2013). In the recent clinical trial on advanced gastrointestinal cancer, patients were given three treatment cycles with modified MSC followed by the prodrug administered 48-72 h later (von Elnem, et al., Int J cancer, 2019). In parallel, 4 days after the administration of a modified neural stem cells, prodrug (5FC) was administered and the modified cells were functional during the entire 7-day course of 5-FC (Portnow, Clinical Cancer Research: An Official Journal of the Americal Association for Cancer Research, 23, 2951-2960, 2017). Hence, it is contemplated that the transiently transfected AT-MSCs with prolonged expression of CD::UPRT herein (see FIG. 7) may be effective over the duration of a treatment regime.

In order to reduce toxicity due to prolonged exposure to the polyplex, a low speed spinning step was used (Ho et al., Nucleic Acids Research, 45(e38), 2017; Boussif, et al., Gene Ther, 3, 1074-1080, 1996). In the present study, we describe an in vitro, non-viral process for engineering theranostic AT-MSCs for GDEPT with high efficacy and high cell viability using cationic polymer. We showed that, despite the high over expression of the transgene, the phenotypic characteristics and migration ability of the modified cells were comparable to the native MSCs. These cells were highly efficient in inhibiting proliferation of cancer cells in vitro. Hence, this process to modify AT-MSCs may provide an effective and safer alternative to viral transduction for stem cell-based cancer therapy, and may be useful for a wide range of applications.

Mesenchymal stem cells (MSCs) have emerged as promising vehicles for gene-directed enzyme prodrug therapy (GDEPT). The therapeutic potency may be improved by using augmented MSCs preconditioned with cytokines and/or growth factors, abiotic conditions, pharmaceuticals, and/or modified genetically and/or reprogrammed, for example. The tumour-trophic properties of MSCs indicate these vehicles to deliver effective, targeted therapies to tumours and metastatic diseases. A key step in modifying MSCs is the delivery of genes with high efficiency and low cytotoxicity. Due to the poor efficiency of traditional transfection approaches, viral methods have been used to transduce MSCs in preclinical and clinical studies. Results herein demonstrate the efficient transfection (>80%) of human adipose tissue derived MSCs (AT-MSCs) using a cost-effective Polyethylenimine, in the presence of fusogenic lipids and histone deacetylase 6 inhibitor. Notably, the cellular phenotypes of MSCs remained unchanged after modification. AT-MSCs modified with a fused transgene, yeast cytosine deaminase::uracil phosphoribosyltransferase (CDy::UPRT), exhibited strong cytotoxic effects towards glioma, breast and gastric cancer cells in vitro. The efficiency of eliminating gastric cell lines were effective even when using 7-day post-transfected AT-MSCs, indicative of the sustained expression and function of the therapeutic gene. Moreover, significant regression of s.c. tumor was achieved by direct injection of single dose therapeutic MSC. Provided herein are efficient modification processes for MSC-based prodrug therapy, as an alternative to the use of viral vectors.

In the present study, processes to modify AT-MSCs at high efficiency using cationic polymer in combination with Enhancer are developed and described, enabling theranostic MSCs producing CDy::UPRT without the need to use virus nor the need to establish stable cell lines. Furthermore, these MSC modification processes are donor agnostic and may be used in a wide range of applications.

Methods and Materials

Cell Culture

Human adipose tissue derived mesenchymal stem cells (AT-MSCs, RoosterBio) was isolated from female donor (LOT00088, age 18-30). AT-MSC was maintained in the hMSC High Performance Basal Media (Roosterbio). Breast cancer cell line MDA-MB-231 (HTB-26, ATCC), and primary human dermal fibroblast (ATCC, PCS-201-012), were cultured and maintained according to manufacturer's instruction. Glioma cell line U-251MG was kindly provided by Paula Lam (Duke NUS Medical School). U-251MG cell line was cultured in DMEM (Dulbecco Modified Eagle Medium) supplemented with 10% Fetal Bovine Serum (FBS, Biowest). Gastric cancer cell line MKN1 and MKN28 was kindly provided by Dr. Yong Wei Peng (National University Cancer Institute, Singapore). The gastric cancer cell lines were cultured in RPMI (Roswell Park Memorial Institute medium, Thermo Scientific), supplemented with 10% FBS. Cells were kept at 37° C. in humidified atmosphere and 5% CO2.

Construction of Cpg Free Expression Plasmid Containing CD::UPRT

Plasmid DNA (pDNA) expressing fused cytosine deaminase and uracil phosphoribosyltransferase (4265 bp pSELECT-zeo-FcyFur (https://www.invivogen.com/pselect-zeo-fcyfur)) was purchased from InvivoGen. Construction of CpG free expression plasmid of CD::UPRT was performed by cross-lapping in vitro assembly (CLIVA) cloning techniques as described [50]. Briefly, Lucia in the plasmid pCpGfree-Lucia (InvivoGen) was replaced with CD::UPRT using pSELECT-zeo-FcyFur as the template in polymerase chain reaction (PCR) (https://www.invivogen.com/pcpgfree). All pDNA were propagated in Escherichia coli DH5a GT115 strain (InvivoGen) under the selection of antibiotic Zeocin as instructed. The plasmids were purified with E.Z.N.A. endo-free plasmid maxi kit according to manufacturer's instruction (Omega Bio-tek).

Transfection Procedure

General Centrifugation Method:

Transgene of interest was introduced into AT-MSCs at passage 3-5. For each well (6-well plate format), 5 mg/mL of LPEI (PEI MAX, Polyscience) was added to pDNA in serum free DMEM at different ratio of pDNA and PEIMAX. In certain embodiments, the N/P ratio may range from 5-100, depending on polymer selected. The mixture, at a total volume of 100 μl, was incubated at room temperature for 15 min. The pDNA:LPEI ratio was calculated according to the amount of pDNA, μg: volume of 1 mg/mL of LPEI, μl. LPEI/pDNA complex was then added to serum free DMEM medium (1:20) to prepare the transfection mixture. The culture media was removed and replaced with the transfection mixture, followed by mild centrifugation at 200 g for 5 min. After centrifugation, the transfection mixture was removed and replaced with complete media, with or without supplementation of TrafEn. TrafEn consist of DOPE/CHEMS (Polar Avanti Lipid) and Vorinostat (SAHA, Bio Vision). The ratio was 9:2, and SAHA is used at 1.25 uM. Cells were incubated for 24 h before analysis.

The above protocol is generally exemplary/representative of the methods used for experiments presented in FIGS. 1-16, 20, and 23, allowing for variations and modifications depending on the particular experiment.

General Non-Centrifugation Method:

Cell preparation: Cell Expansion—Culture media was taken out of the refrigerator and warmed at room temperature. MSC vial (0.5M cells/vial) was obtained from liquid nitrogen dewar and immediately thawed in 37° C. water bath by rigorous agitation. Note: In certain embodiments, MSC may be obtained from different sources/species/donors. Vial well was sprayed with 70% alcohol before transferring into biosafety cabinet. Cells were aseptically transferred into a 15 mL centrifuge tube. 4 mL of culture media was added slowly (dropwise) to the cells. Centrifuge at 200×g was performed for 5 min. The supernatant was carefully removed without disturbing the cell pellet. Cells were resuspended in 10 mL of culture media. Mix well and seed cells into 1×T75 vessel. Cell growth was observed using microscope to ensure culture reaches >80% confluency before harvesting. Cells were ready for use 2 days later.

Cell preparation: Cell Growth—To harvest cells, vessel was transferred into biosafety cabinet and spent media removed. Media was removed and rinsing once with 3 mL 1×PBS was performed. The wash solution was aspirated. 2 mL of Accutase was added to the flask, and incubation in 37° C. (3-5 min) was performed. Gentle tapping was used to dislodge remaining cells from surface of the flask. 4 mL of fresh culture media was added to quench Accutase activity. The cell suspension was transferred into a 15 mL centrifuge tube. Centrifugation at 200×g for 5 min was performed. The supernatant was aspirated and resuspended cells with 10 mL of fresh media. Mix well and transfer 0.1 mL of cells into microcentrifuge tube for cell counting. Cell count should be in the range of 0.1-1×10⁶ cells/mL. Cell suspension may be subcultured, frozen, or used for generation of CD::UPRT expressing MSC. For subculture, seed 5000-7000 cells/cm² of cell culture surface. Top up with fresh media accordingly. Cells may be subcultured up to 6 passages. For cryopreservation, centrifuge cells at 200×g for 5 min. Aspirate the supernatant and resuspend cells in KBM Banker 2 at 1M cells/mL. Aliquot 500 μL of the suspension cells to each vial.

Generation of CD::UPRT expressing MSC: Cell Seeding—The optimal confluency for MSC is ˜60%. Cells were seeded at 24 hours prior to transfection. Note: The TrafEn™ reagents are stable in the presence of serum and antibiotics. Standard culture medium may be used during the entire experiment. Recommended number of cells to be seeded 24 hours prior to transfection in at least 2×T175 for preparation of 10M cells:

| Cell type | Cell density | Culture media |
|---|---|---|
| Any MSC type | 2-3 M in 1xT175 | 20 mL |

*Cell number may vary due to the different growth rate of MSC.

Generation of CD::UPRT expressing MSC: DNA transfection protocol

Preparation of reagent:

Fusogenic lipid, first agent: provided as a 1× working stock. HDACi, second agent: Dissolve HDACi in DMSO. Aliquot and store the diluted solution at −20° C.

Step 1: Complexation

Dilute 9-50 μg of DNA in 1500 μL of complexation buffer. Vortex for 5 sec to mix. Add cationic polymer to the diluted DNA. Vortex for 5 sec to mix. *1.5-30 ug of polymer to 1 ug of DNA. Incubate the transfection mixture for 15 min at room temperature.

Step 2: Preparation of TrafEn mixture

During the incubation of transfection mixture, combine 0.2-1 mg of first agent and second agent. Mix immediately by pipetting. Do not vortex. Incubate for 10-20 min at room temperature.

Step 3: Transfection

Add 2500 μL fresh culture media to the transfection reagent/DNA mixture. Add 4200 μL transfection reagent/DNA mixture drop-wise to the culture vessel. Do not remove the growth medium from the cells before adding the transfection reagent/DNA.

Add TrafEn mixture drop-wise to culture vessel.

Gently rock the culture vessel back and forth and from side to side to mix.

Return culture vessel to incubator.

Cells were used 24 hours post-transfection.

Generation of CD::UPRT Expressing MSC: Cell Harvesting—

To harvest cells, transfer vessel into biosafety cabinet and remove spent media. Remove media and rinse once with 10 mL 1×PBS. Aspirate the wash solution. Add 5 mL of Accutase to the flask, incubate in 37° C. (3-5 min). Gently tap to dislodge remaining cells from surface of the flask. Add 10 mL of fresh culture media to quench Accutase activity. Transfer the cell suspension into a 15 mL centrifuge tube. Mix well and transfer 0.1 mL of cells into microcentrifuge tube for cell counting—Total cell number for each flask may be ˜5M. Centrifuge at 200×g for 5 min. Aspirate the supernatant and resuspend cells with 10 mL of 1×PBS. Centrifuge at 200×g for 5 min. Aspirate the supernatant and resuspend cells with 10 mL of 1×PBS. Centrifuge at 200×g for 5 min. Aspirate the supernatant. Resuspend cells in the remnant of PBS. Cells are ready for use.

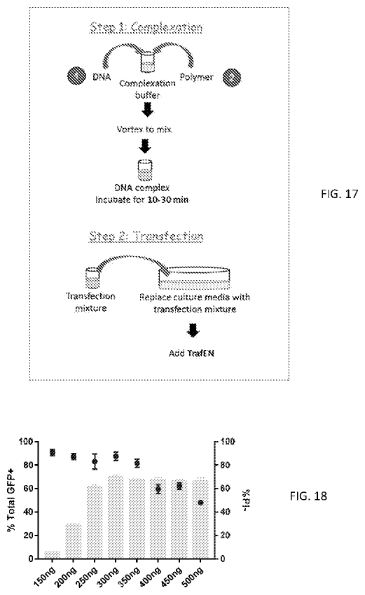

The above protocol is generally exemplary/representative of the methods used for experiments presented in FIGS. 17-19, 21-22, 24, and 26-27, allowing for variations and modifications depending on the particular experiment. Experiments presented in the identified figures were typically performed in culture vessels of about 1.9 to about 75 cm².

In certain embodiments, non-centrifugation methods such as those described above may be suitable for large scale MSC modification. For example, large scale operations of about 175 cm² surface area may be amenable to such non-centrifugation methods.

Expression Analysis

Flow cytometry, western blot and immunocytochemistry were performed as previously described [49].

Flow cytometry: Percentage of fluorescence positive cells was quantified by Attune NxT Flow Cytometer system (ThermoFisher Scientific) and the raw data was analysed using Invitrogen Attune NxT software (ThermoFisher Scientific).

Imaging: Cell images were taken with EVOS FL Cell Imaging System (ThermoFisher Scientific) equipped with three fluorescent light cube for viewing of DAPI (Ex357/Em447), GFP (Ex470/Em510) fluorescence.

Western blot: Samples were analysed by immunoblotting technique with sheep anti-CDy (PA185365, ThermoFisher Scientific) and monoclonal anti-β-Actin (A2228, Sigma-aldrich), respectively.

Immunocytochemistry: The samples were labelled with sheep anti-CDy and Alexa Fluor 488 donkey anti-sheep fluorescent secondary antibody (A11015, ThermoFisher Scientific). Image acquisition was performed using the EVOS FL Cell Imaging System. All images were taken with identical optical settings.

Characterization and Differentiation Potential of CDy::UPRT Producing AT-MSCs

To examine the phenotype of CDy::UPRT producing AT-MSCs, cells were labelled with MSC Phenotyping Kit consisting of antibodies CD73, CD90, CD105, CD14, CD20, CD34, CD45, and HLA-DR (Miltenyi Biotech) according to manufacturer's instructions. After which, expression of the markers were analysed with FACS. High quality MSC population consist of >95% CD90, CD105, and CD73 positive cells. The population expressing CD14, CD20, CD34, CD45, and HLA-DR would be less than 1% [51]. The multipotency of AT-MSCs was confirmed by its differentiation capacity into osteogenic and adipogenic lineage [52, 53]. Differentiation of AT-MSCs was induced with StemPro™ Osteogenesis Differentiation Kit and StemPro™ adipogenesis Differentiation Kit (ThermoFisher Scientific). Unmodified AT-MSCs were used as control. The phenotype and differentiation potential of CDy::UPRT producing AT-MSCs should not vary significantly from the unmodified AT-MSC.

Cell Viability Assay

Quadruplicates of AT-MSC, MKN1, MKN45, MDA-MB-231 (10,000 cells per well) and U-251MG (4000 cells per well) for each treatment were plated into 96-well plates. Twenty four hours later, culture medium was replaced for medium containing various concentration of 5-Fluorocytosine (5-FC, InvivoGen) or 5-Fluorouracil (5FC, InvivoGen). One to five days later, plates were subjected to the CellTiter 96 Aqueous One Solution Cell Proliferation Assay (Promega). The colorimetric read out was measured spectrophotometrically at 490 nm. Results were expressed as the percentage of cell viability, in relative to cells in condition without 5-FC or 5-FU (set to 100%).

In Vitro Drug Susceptibility

Quadruplicates of AT-MSC, MKN1, MKN45, MDA-MB-231 (10,000 cells per well) and U-251MG (5,000 cells per well) for each treatment were plated into 96-well plates. Twenty-four hours later, culture medium was replaced for medium containing various concentration of 5-Fluorocytosine (5-FC, InvivoGen) or 5-Fluorouracil (5FC, InvivoGen). One to five days later, plates were subjected to the CellTiter 96 Aqueous One Solution Cell Proliferation Assay (Promega). The colorimetric read out was measured spectrophotometrically at 490 nm. Results were expressed as the percentage of cell viability, in relative to cells in condition without 5-FC or 5-FU (set to 100%).

Anticancer Efficacy of CDy::UPRT Producing AT-MSCs In Vitro

Direct co-culture: Quadruplicates of gastric cancer cell lines and breast cancer cell line (5000 cells) and U-251MG (2000 cells) were plated in 96-well plates. Five hours later, increasing numbers of either unmodified or CDy::UPRT-producing AT-MSCs at the ratios of 1 AT-MSC to 1, 5, 10, 50 and 100 cancer cells were added to the cancer cell culture. One day later, the culture media was replaced with DMEM supplemented with 2% FBS, with or without 5-FC (0-150 μg/mL). Five days later, cell viability was measured by proliferation assay (commercial assay—https://www.promega.sg/products/cell-health-assays/cell-viability-and-cytotoxicity-assays/celltiter-96-non_radioactive-cell-proliferation-assay-_mtt_/?catNum=G4000). Conditions without 5-FC was set to 100%.

Indirect Coculture: MB-MDA-231 cells were plated on 24-well plate (5×10⁴ cells per well). AT-MSCs or CDy::UPRT_AT-MSCs (5×10⁴ cells per well) were plated on transwell (Corning, C05/3422). After 6 h of cultivation, inserts with therapeutic cells were transferred into the wells with MB-MDA-231 cell line, with or without 5FC. Cytotoxic effect was evaluation after 4 days of incubation. Transwells were removed and culture media was replaced with 1×PBS containing 1 μg/mL of Hoechst 3222. Stained cells were analysed using Synergy H1 microplate reader at excitation and emission wavelength of 358 nm and 461 nm, respectively. With gain setting at 80, RFU at 9 areas of the cell culture were recorded. Proliferation inhibition after treatment will be calculated relative to the control (coculture of untransfected AT-MSC and MB-MDA-231 cells).

Anticancer Efficacy of CDy::UPRT Producing AT-MSCs In Vivo

Five to six-week old female nude mice were purchased from InVivos and used for the in vivo studies under IACUC approved protocol (R18-1383). Mice were anesthetized by isoflurane inhalation and 5×10⁶ Temozolomide resistant U-251MG cells suspended in 100 μl DMEM (50% Matrigel) were injected s.c. in dorsal flank regions (one tumor per mouse). The growth of tumour was monitored by digital caliper. When tumors measured an average volume of 80-200 mm³ treatment was started. All mice were randomly distributed into 3 groups each containing 5 mice. Prodrug control group received daily injections of prodrug. Cell control group received intratumoral injection of 1×10⁶ MSCs plus daily injections of prodrug. Treatment group received intratumoral injection of 1×10⁶ CD::UPRT_AT-MSCs plus daily injections of prodrug. Modified or non modified MSC were administrated intratumorally on day 0 (single dose). One day later, mice received i.p. administration of 500 mg/kg of 5FC for 4 consecutive days. Before cell injection (Day 0) and Day 7, 11 and 15 after MSC administration, tumor sizes and body weights were measured.

Cell Invasion Assay

The tumour tropism of AT-MSCs was determined using BD Biocoat™ matrigel invasion chambers (BD Biosciences). Cancer cell lines or HEK293T cells were loaded in the lower well of the 24-well plates. Twenty four hours later, unmodified and CDy::UPRT-producing AT-MSCs in serum-free DMEM were added onto the invasion chambers. Lower wells were washed with 1×PBS, filled with serum free DMEM, assembled for the invasion assay. After 24 h incubation, non-invading cells and matrigel were removed from the inside of the insert.

Invaded cells were stained with Hoechst 33342 (ThermoFisher Scientific) and photographed through the imaging system. Number of cells in 3 frames were counted.

Statistical Analysis

Where Student's t-test, was used, an unpaired two-tailed test was used, with the assumption that changes in the readout are normally distributed.

Abbreviations

MSC: mesenchymal stem cell; PEI: polyethylenimine; HDAC6i: histone deacetylase 6 inhibitor; CD::UPRT: fused yeast cytosine deaminase::uracil phosphoribosyltransferase; CD: cytosine deaminase; GDEPT: gene-directed enzyme prodrug therapy; UPRT: uracil phosphoribosyl-transferase; FUMP: 5-fluorouridine monophosphate; 5FC: 5 fluorocytosine; 5FU: 5 fluorouracil; DPD: Dihydropyrimidine dehydrogenase; OPRT: Orotate phosphoribosyltransferase; GFP: Green Fluorescence protein; AT-MSC: human adipose tissue derived MSC; DMEM: Dulbecco Modified Eagle Medium; FBS: Fetal Bovine Serum; pDNA: Plasmid DNA; MOI: Multiplicity of infection; HSV-TK: Herpes Simplex Virus-1 Thymidine Kinase.

### Example 2—Scalable Methods for High Efficiency Transfection of MSCs

Processes that omit centrifugation may improve scalability. However, this may result in extended incubation times and present a challenge if the cationic polymer is not appropriately selected. Process development may be desirable to optimize the protocol for each MSC donor/type. In the present studies, we have identified important steps (FIG. 17) to enable the generation of clinically useful modified MSCs for therapeutic purposes. Examples of desirable features of the protocol are detailed in Table 5.

TABLE 5
Examples of desirable features

| | |
|---|---|
| 1 | No requirement of low speed centrifugation |
| 2 | Low cytotoxicity post transfection |
| | (>70% viable cells based on Propidium iodide assay) |
| 3 | >70% number of cells transiently transfected |
| 4 | No requirement for selection of stably transfected cells |
| 5 | Retention of MSC phenotypic characteristics |
| 6 | Scalable production |
| 7 | Prolonged expression |

Scalability

An important aspect for clinical application of MSC prodrug gene delivery is production scalability of the gene delivery method. Previous reports of TrafEn to enhance centrifugation based polymer transfection had limited scalability. The present studies describe the addition of polyplexes and TrafEn directly into any culture vessels, without the need of centrifugation. A particular difference with the present development is the duration of exposure to polymers and TrafEn. In these studies, the suitable formulation of polymer and TrafEn may be incubated with the cells without the need to centrifuge. This may provide a means to scale in production as the need to centrifuge large containers will not be necessary nor convenient (FIG. 24A-B). It is worthy to note that the transfection efficiency remained unchanged regardless of the surface area of the cell culture, suggesting the feasibility of translating the enhancing effect of TrafEn to preclinical and clinical scale.

In order to scale in production of these highly transfected MSC, the compatibility of cationic polymers and TrafEn was performed and is desirable. In an examplary process development (FIG. 17), various linear (LPEI) and branched (BPEI), of a range of molecular weights (MW), from <4 to 200 kDa, were evaluated for high transfection efficiencies and low cytotoxicity without the need for centrifugation. Depending on the MSC type, donor and culture conditions, the most suitable compositions may be selected and augmentation then carried out using TrafEn (as in FIG. 20, for example). Hence, an important step is the identification of these compatible polymers that may be augmented with TrafEn.

In certain embodiments, polymer type, polymer structure (linear, branched), and/or polymer size may be selected or tailored to the particular cell type. By way of example, results in FIG. 20 indicate that MSCs in FIG. 20A prefer large polymer, while MSCs in FIG. 20B prefer small polymer. In certain embodiments, the MSCs may comprise UC-MSC, and the polymer may comprise a polymer greater than about 50 kDa, between about 50 and about 200 kDa, or greater than about 200 kDa. In certain embodiments, the MSCs may comprise BM-MSC, and the polymer may comprise a polymer smaller than about 50 kDa, between about 50 kDa and 5 kDa, or smaller than about 5 kDa. In certain embodiments, the polymer may comprise LPEI.

Low Toxicity and High Efficiency.

Referring to FIG. 26, for successful use of MSC in prodrug therapy, the health of cells is desirable to ensure the functionality of MSC during treatment. At DNA amount lower than 350 ng, maximal transfection efficiency (>90%) was achieved without signification reduction in cell viability, as indicated by propidium iodine exclusion assay (FIG. 26).This data suggests the robustness of embodiments of the workflow in modifying MSC at high efficiency without introducing significant cytotoxicity in MSC culture.

DNA Optimization (1 in FIG. 17)

DNA Amount

High amounts of DNA may result in cytotoxicity. The range of DNA used for MSC transfection is shown ranging from 100 to 500 ng for surface area of 1.9 cm². As shown in FIG. 18, increasing DNA amount was not beneficial due to the high cytotoxicity.

DNA Vector Design

Using a suitable DNA vector design may prolong transgene expression. An aim for generation of stable cell lines is to obtain high numbers of transfected cells. Antibiotic selection is highly labor intensive (2-3 weeks) and may potentially compromise MSC quality [17], cause cell senescence [17] and reduce tumour tropism [18] as well as safety concerns with viral induced MSC transformation [19]. Without antibiotic selection, poor transfection efficiency may not be sufficient for clinical application with traditional approaches.

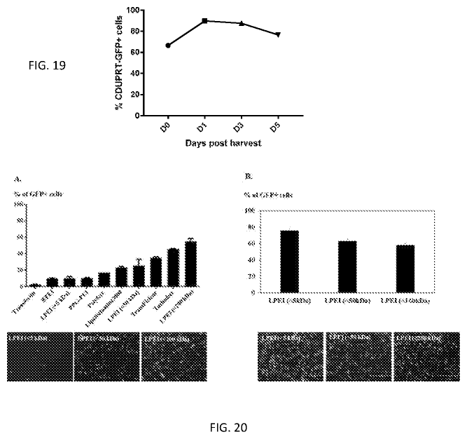

In view of the successful high transfection using the processes developed and described herein, it may not be necessary to generate stable cell lines. With the therapeutic gene subcloned into a CpG free vector, the high expression of CDy::UPRT over a 7 day period was comparatively effective in eliminating cancer cells as those 1 day after transfection (FIG. 7). Prolonged expression was observed using a transfection protocol free of centrifugation (FIG. 19). The expression of CDy::UPRT reduced significantly overtime, resulting in reduction of the anti-cancer efficacy of the modified MSC (FIG. 16).

In addition to removal of one or more CpG islands from plasmid construct, the present methods were compatible to use of other plasmids known in the art, for example vectors listed in: Jackson D A, et al. Designing Nonviral Vectors for Efficient Gene Transfer and Long-Term Gene Expression, Molecular Therapy, 14:613-26, which is incorporated by reference in its entirety. Suitable vectors may include those that have been shown to result in prolonged expression such as: Scaffold/matrix attachment regions (S/MARs), Episomal vectors, and EBNA-1 containing vectors.

Polymer Optimization (2 in FIG. 17)

In embodiments of this invention, a suitable polymer may be incubated with the cells without the need to centrifuge. This may provide means to scale in production as the need to centrifuge large containers will not be necessary nor convenient. Prolonging the exposure and incubation time of cells and transfection mixtures greater than 20 minutes may introduce cytotoxicity. Certain polymers may exhibit cytotoxicity under certain conditions. For instance, Ho, et al., Enhanced transfection of a macromolecular lignin-based DNA complex with low cellular toxicity, Biosci. Rep. (2018) 38:1-9 encountered toxicity with Lignin-PGEA-PEGMA.

Selection of a compatible polymer with cell type may be desirable to ensure higher quality and higher transfection efficiency. Various linear (LPEI) and branched (BPEI), of a range of molecular weights (MW), from 2 to 200 kDa, were evaluated for high transfection efficiencies and low cytotoxicity without the need for centrifugation. Depending on the MSC type, donor and culture conditions, the most suitable compositions were selected and augmentation was then carried out using TrafEn. For example, FIG. 19 shows that LPEI <200 kDa and <5 kDa were compatible with umbilical cord MSC (UC-MSC) and BM-MSC, respectively.

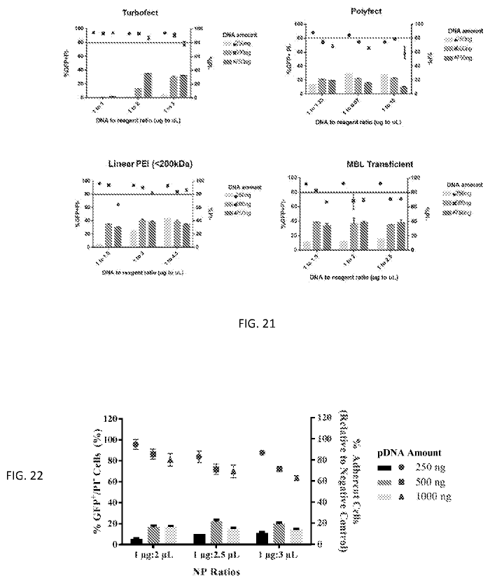

In addition to the compatibility of polymer, optimization of the amount of polymer may increase cell viability. Cytotoxicity is highly associated with polymer and DNA amount, increasing amount resulted in a higher toxicity, as indicated by the lower % of Propidium Iodide (PI) negative cells. When the content and concentration of commercial polymer is unknown (for example with reagents such as Turbofect, Polyfect and Transficient), reduced cell viability was observed with increasing polymer amount. For PEI based polymers, we tested amounts of polymer ranging from 1 ug-30 ug for 1 ug of DNA (FIGS. 20, 21).

These studies resulted in an example method for High Efficiency Transfection of MSCs which may include the following steps.

Cell Preparation

Cell Expansion: Take culture media out of the refrigerator and warm it at room temperature. Obtain MSC vial (0.5M cells/vial) from liquid nitrogen dewar and immediately thaw in 37° C. water bath by rigorous agitation. MSC can be obtained from different sources/species/donors. Spray vial well with 70% alcohol before transferring into biosafety cabinet. Aseptically transfer cells into a 15 mL centrifuge tube. Add 4 mL of culture media slowly (dropwise) to the cells. Centrifuge at 200×g for 5 min. Remove the supernatant carefully without disturbing the cell pellet. Resuspend the cells in 10 mL of culture media. Mix well and seed cells into 1×T75 vessel. Observe cell growth using microscope to ensure culture reaches >80% confluency before harvesting. Cells are ready for use 2 days later.

Cell Growth: To harvest cells, transfer vessel into biosafety cabinet and remove spent media. Remove media and rinse once with 3 mL 1×PBS. Aspirate the wash solution. Add 2 mL of Accutase to the flask, incubate in 37° C. (3-5 min). Gently tap to dislodge remaining cells from surface of the flask. Add 4 mL of fresh culture media to quench Accutase activity. Transfer the cell suspension into a 15 mL centrifuge tube. Centrifuge at 200×g for 5 min. Aspirate the supernatant and resuspend cells with 10 mL of fresh media. Mix well and transfer 0.1 mL of cells into microcentrifuge tube for cell counting. Cell count should be in the range of 0.1-1×106 cells/mL. Cell suspension can be subcultured, frozen, or used for generation of CD::UPRT expressing MSC. For subculture, seed 5000-7000 cells/cm² of cell culture surface. Top up with fresh media accordingly. Cells can be subcultured up to 6 passages. For cryopreservation, centrifuge cells at 200×g for 5 min. Aspirate the supernatant and resuspend cells in KBM Banker 2 at 1M cells/mL. Aliquot 500 μL of the suspension cells to each vial.

Generation of CD::UPRT Expressing MSC

Cell Seeding: The optimal confluency for MSC is −60%. Cells are seeded at 24 hours prior to transfection. Note: The TrafEn™ reagents are stable in the presence of serum and antibiotics. Standard culture medium can be used during the entire experiment.

DNA Transfection Protocol

Preparation of reagent: Fusogenic lipid, first agent: provided as a 1× working stock. HDACi, second agent: Dissolve HDACi in DMSO. Aliquot and store the diluted solution at −20° C.

Step 1: Complexation

Dilute 9-50 μg of DNA in 1500 μL of complexation buffer. Vortex for 5 sec to mix. Add cationic polymer to the diluted DNA. Vortex for 5 sec to mix. *1.5-30 ug of polymer to 1 ug of DNA. Incubate the transfection mixture for 15 min at room temperature.

Step 2: Preparation of TrafEn Mixture

During the incubation of transfection mixture, combine 0.2-1 mg of first agent and second agent. Mix immediately by pipetting. DO NOT VORTEX! Incubate for 10-20 min at room temperature.

Step 3: Transfection

Add 2500 μL fresh culture media to the transfection reagent/DNA mixture. Add 4200 μL transfection reagent/DNA mixture drop-wise to the culture vessel. Do not remove the growth medium from the cells before adding the transfection reagent/DNA. Add TrafEn mixture drop-wise to culture vessel. Gently rock the culture vessel back and forth and from side to side to mix. Return culture vessel to incubator. Cells are ready for use 24 hours post-transfection.

Cell Harvesting: To harvest cells, transfer vessel into biosafety cabinet and remove spent media. Remove media and rinse once with 10 mL 1×PBS. Aspirate the wash solution. Add 5 mL of Accutase to the flask, incubate in 37° C. (3-5 min). Gently tap to dislodge remaining cells from surface of the flask. Add 10 mL of fresh culture media to quench Accutase activity. Transfer the cell suspension into a 15 mL centrifuge tube. Mix well and transfer 0.1 mL of cells into microcentrifuge tube for cell counting—Total cell number for each flask should be ˜5M. Centrifuge at 200×g for 5 min. Aspirate the supernatant and resuspend cells with 10 mL of 1×PBS. Centrifuge at 200×g for 5 min. Aspirate the supernatant and resuspend cells with 10 mL of 1×PBS. Centrifuge at 200×g for 5 min. Aspirate the supernatant. Resuspend cells in the remnant of PBS. Cells are ready for use.

### Example 3: Developing Transfection Methods and Processes for any of a Variety of MSC Cell Types

As described in FIG. 17, process optimization of one or multiple steps may be desirable to empirically identify the conditions for TrafEn to enhance MSC transfection. We found that the method provided increased expression duration, scalability and quality of MSC post modification of MSC modified at >70% efficiency.

Different MSC types may be efficiently transfected. Various types of MSC have been used for cancer therapy (Table 2). The current methods have been validated in various MSC types (Bone marrow, adipose derived, umbilical cord) for prodrug gene therapy (FIG. 14B). Embodiments of the methods described herein may be used for any of the MSC types such as adipose MSC, BM MSC (such as Roosterbio), UC MSC (such as ATCC, cell applications), cord lining MSC (such as cell research corporation).

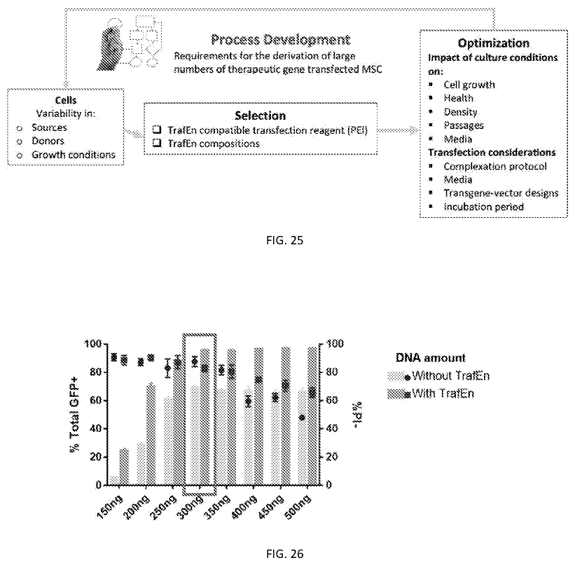

Process Development for any of a Variety of MSCs (FIG. 25)

Cell Variability

Referring to FIG. 14B, comparable transfection efficiency and anticancer efficiency in stem cells from different sources are shown. Adipose tissue (AT, Roosterbio), bone marrow (BM, Roosterbio), and UC (Umbilical cord, ATCC) derived MSCs were transfected with the centrifugation protocol in the presence of TrafEn. Twenty four hour post transfection, cells were trypsined and collected for western blot analysis (FIG. 14A). The cells were lysed for immunoblotting analysis with antibody targeting CDy and Actin. In the same experiment, cells were harvested for coculture study with various cancer cell lines at the ratio of 1 MSC to 50 cancer cells (FIG. 14B). Cells were cocultured in the media containing 100 μg/mL of 5FC for 5 days. At the end of incubation, remaining cell number was evaluated spectrophotometrically by measuring the RFU of cells stained with Hoechst 33342 at wavelength Ex340/Em488. Conditions with unmodified MSCs serve as control. Percentage of proliferation inhibition was calculated according. Graph represents data collected from quadruplicates, mean+SEM.

TrafEn Selection

In certain embodiments, TrafEn selection may be based on [1] transfection efficiency, [2] cell viability, or both.

In certain embodiments, TrafEn compatible transfection agents may be selected/optimized by screening a library of polymer, which may include commercially available polymers such as Turbofect (ThermoScientific), Jetprime (Poplyplus transfection). PEI is an example of a polymer identified herein.

Nucleic Acid Construct Selection

DNA design and quantities may be selected and optimized based on the particular application. In certain embodiments, DNA design may be based on [1] duration of expression, [2] transfection efficiency, or both. DNA quantity may be optimized based on [1] transfection efficiency, cell viability, or both.

Cationic Polymer Selection

Cationic polymer may be selected and optimized based on the particular application. In certain embodiments, cationic polymer may be selected and optimized by screening a library of available polymers ranging from about 4 to about 200 kDa in size.

Polymer ratio may be selected and optimized based on the particular application. In certain embodiments, polymer ratio may be selected and optimized by testing within a range of N/P of about 5-100.

In certain embodiments, polymer type and ratios may be selected based on a balance of transfection efficiency and cell viability.

In certain embodiments, for a particular gene-based application, selection and/or optimization may be performed and may include polymer screening, screening of DNA amount, and polymer/DNA ratio screening. Outcomes of screening may determine the protocol/workflow based on outcome of [1] transfection efficiency >70%, [2] cell viability >70%.

Optimizations

Culture Conditions

Cell Growth (Seeding): The optimal confluency for MSC may be ˜60%. MSCs may be seeded at 24 hours prior to transfection. Note: The TrafEn™ reagents are stable in the presence of serum and antibiotics. Standard culture medium can be used during the entire experiment.

Cell Health: Post transfection, the cell viability is preferably >about 70%, as defined by propidium iodide assay. The cell quantity should preferably not be significantly compromised, in terms of differentiation potential and phenotypic markers.

Cell density: Call density may typically range from about 60-90% in certain embodiments. Optimization may be performed to examine transfection at density of about 60%, about 70%, about 870%, or about 90%, for example.

Passages: In certain embodiments, efficiency may be consistent for MSC from passages 1-25, as long as MSCs are not scenescent.

DNA Transfection Optimization:

Complexation: By way of example, dilute 9-50 μg of DNA in 1500 μL of complexation buffer. Vortex for 5 sec to mix. Add cationic polymer to the diluted DNA. Vortex for 5 sec to mix. *1.5-30 ug of polymer to 1 ug of DNA. Incubate the transfection mixture for 15 min at room temperature. Optimization may be on DNA amount. For surface area of about 1.9 cm², the DNA amount may vary from about 100 to about 500 ng. DNA amount higher than about 500 ng may, in certain embodiments, be toxic to stem cells in certain examples.

Preparation of TrafEn mixture: During the incubation of transfection mixture, combine 0.2-1 mg of first agent and second agent, for example. As will be understood, in certain embodiments, the amount and/or ratio of first and second agent may be varied based on cell type being used. For example, in certain embodiments, ratio of fusogenic lipid and helper lipid may be varied. The second agent may comprise, for example, HDACi, which may target HDAC6. First and second agents may include those described in WO2014/070111, which is herein incorporated by reference in its entirety.

Transfection Media: By way of example, add 2500 μL fresh culture media to the transfection reagent/DNA mixture. Transfection media may be used for preparation of DNA-polymer complex. DNA and polymer may be added to the transfection media, and incubated for about 10 to about 45 minutes, for example. Post incubation, the transfection mixture may be added directly to the cell culture.

Transgene-vector designs: Transgene vectors may vary. Generally, transgene vectors may comprise a promoter and a transgene. For extended or prolonged expression, addition of modules such as codon optimization to remove CpG islands, S/MAR, and/or promoter optimization may be used.

Incubation period: In certain embodiments, incubation may be performed at about 37 degrees Celsius, for about 2 to about 48 hours, or about 2 to about 24 hours, for example.

The range of various conditions including DNA amount, polymer, cell density and TrafEn formulation may be determined accordingly, and may be tailored for the MSC source. After process optimization, the product may comprise a specific workflow/protocol and optimized reagents (plasmid DNA, polymer, TrafEn) which may be formulated into a kit form in certain embodiments. Such an approach may provide robustness of the process and/or facilitate good reproducibility of the transfection outcome in other laboratories and settings.

### Example 4—Additional Results for Non-Spinning Protocol

A study on the anticancer efficiency of CD::UPRT_MSC on Temozolomide resistant glioblastoma model (in vitro and in vivo data) was performed. MSC source was Adipose tissue derived MSC (AT-MSC) from Roosterbio.

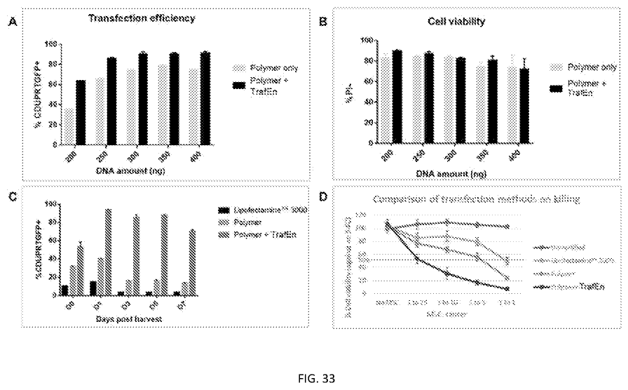

In line with the release criteria for the use of virally modified MSCs for cancer treatment (EU Clinical Trials Register number: 2012-003741-15), a comparable level of cell modification (>75%) and cell viability (>80%) is highly desired with any non-viral gene delivery strategies. Using the TrafEn mediated transfection protocol has shown transfection efficiency of over 90% of with cell viability above 80% (FIGS. 33A & 33B, note that the modified MSC was prepared according to the non-centrifugation protocol described above, although both centrifugation and non-centrifugation protocols can be used). This was above the specified threshold of 80% cell viability based on the TREATME-1 protocol that was recently used in clinical trial (Niess, 2015; von Elnem, 2019). This was achieved two days post-transfection, without the need for antibiotic selection. Additionally, results demonstrated the correlation of transfection efficiency with the ability of modified MSCs in killing cancer cells (FIGS. 33C & 33D). This confirmed that TrafEn can be used to generate a cost-effective, off-the shelf-style, allogenic MSC-GDEPT.

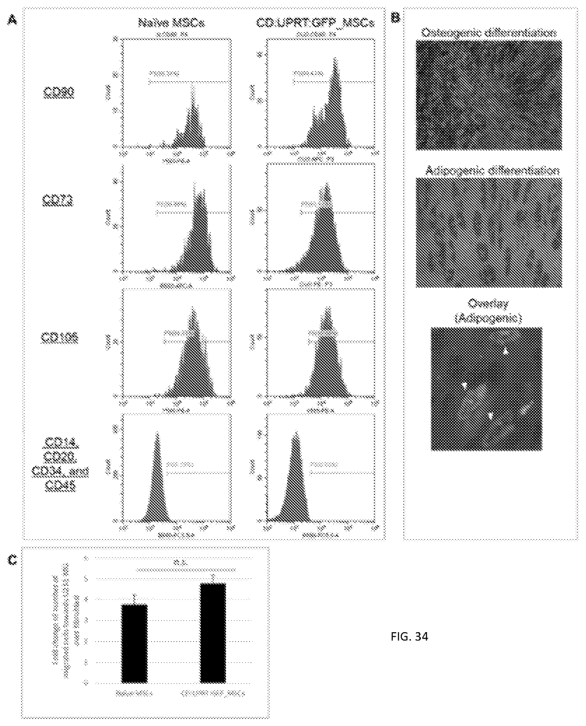

Upon obtaining high number of CD:UPRT:GFP_MSCs, we evaluated the stem cell properties post-modification. Following the characteristics defined by the ISCT (Dominici, Cytotherapy 8, 315-317, 2006), the modified MSCs should retain the CD markers expressions and differentiation potentials. For the CD markers characterization, over 95% of CD:UPRT:GFP_MSCs expressed CD73, CD90 and CD105 and less than 2% of the cells expressed CD14, CD20, CD34, and CD45, similar to naïve MSCs (FIG. 34A). The CD:UPRT:GFP expressing cells were able to differentiate into both osteogenic and adipogenic lineages upon induction (FIG. 34B), similar characteristics to naïve MSCs. Upon modification of the AT-MSCs, we observed no significant difference in tumour tropism between naïve and transfected AT-MSC (FIG. 34C). AT-MSCs were found to migrate specifically to cancer cell line over non-cancerous cells, ie fibroblast. This data suggests TrafEn mediated transfection does not affect the MSC phenotype as cell vehicle for tumour targeting.

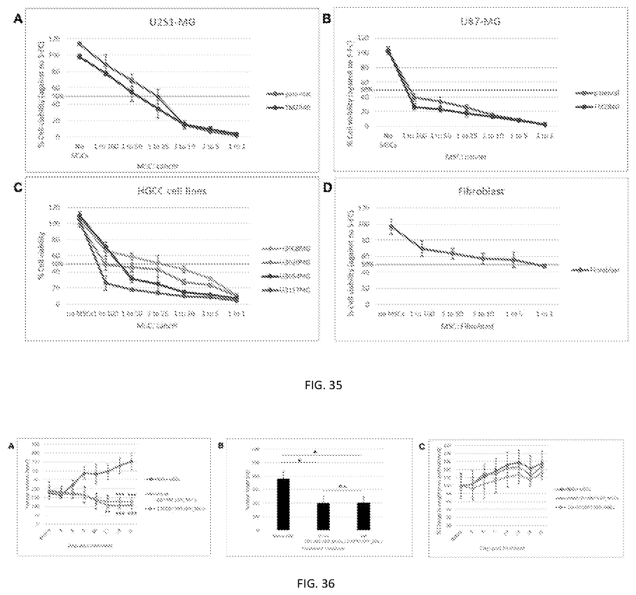

The treatment of Glioblastoma multiforme (GBM) is a huge challenge and an unmet need due to high chance of recurrent GBM with Temozolomide (TMZ) resistance. CDEPT may potentially be provided as second line therapy for TMZ non-responder. Here, we determined if non-viral CD:UPRT:GFP_MSC/5-FC system was effective against Temozolomide (TMZ) resistant glioma cell lines. Both the parental and TMZ resistant U251-MG cell lines were as sensitive towards these modified MSCs with 5-FC (FIG. 35A). The cell viability of the co-culture increased in a MSC-dose dependent manner. Upon reduction in MSC to cancer ratio, a higher cell viability, indicating less killing, was observed. Similar observation was found in U87-MG and U87-MGTMZR40 (FIG. 35B). Next, we determine if this holds true for the HGCC patient derived glioma cell lines which were previously reported to be TMZ resistant. Similarly, we observe efficient killing of the HGCC glioma cell lines (FIG. 35C). To determine the toxicity of the system against non-cancer cells, we examine the cytotoxicity of CD:UPRT:GFP_MSC/5-FC in Fibroblast culture. Interestingly, fibroblast was less sensitive, with 47.78±2.93% even at MSC to fibroblast ratio of 1:1 (FIG. 35D).

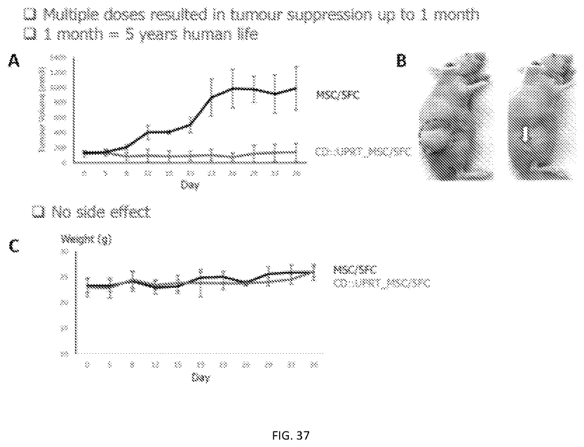

Next, we investigated the in vivo cytotoxicity effect of the prodrug therapy in a subcutaneous mice model using U251-MG^{TMZR40}, the TMZ resistant cells. The CD:UPRT:GFP/MSCs group of mice showed significant decrease in tumour volume with single intra-tumoural administration of cells. The difference between the modified cell treated and naïve cell control groups was significant from as early as 7 days post treatment and sustained over a period of 15 days (FIG. 36A). Tumour was harvested on day 15 post treatment and a significant difference between the weight of tumours between naïve and modified MSCs was observed (FIG. 36B). Interestingly, a dose escalation in the use of modified cells (0.5×10⁶ versus 1×10⁶) showed similar changes in tumour volume (FIG. 36A, grey and light blue circles, & FIG. 36B). An important finding in this study is that there appeared to be no observable systemic toxicity as determined by the changes in the body weight of the animals (FIG. 36C). Long term tumour suppression was observed after the completion of 3 cycles-treatment (FIG. 37).

### Example 5—Large Animal Studies—Multi-Site Investigational Study

A large animal study was performed. Primary objectives of this study, which were achieved, are as follows:

1) No significant side effects

2) Reduction of tumour size

3) Improvement of the quality of life

4) Extension of life

Modified MSC cells were prepared according to the non-centrifugation protocol described above, scaled to 20-30M cells in a 500 cm2 culture vessel.

Animal participation was irrespective of tumour type or age of the dog. Inclusion criteria included an animal with biopsy data. Exclusion criteria included a tumour that could not be sampled or the patient had systemic illness (such as marked fever, immune suppression, organ failure). The anti-cancer efficacy of CDEPT has been demonstrated in dogs and cats presenting with various cancers; including lymphoma (lymph node enlargement), thyroid carcinoma, melanoma, perianal carcinoma, soft tissue sarcoma, nasal carcinoma, gastrointestinal cancer, lymphoma (blood borne). In all cases, blood test was performed before and after completion of each cycle, no significant change in the BUN and ALKP value. Overall, the clinical presentations suggest clinical benefits and good safety profile of CD::UPRT_MSC/5FC as described herein.

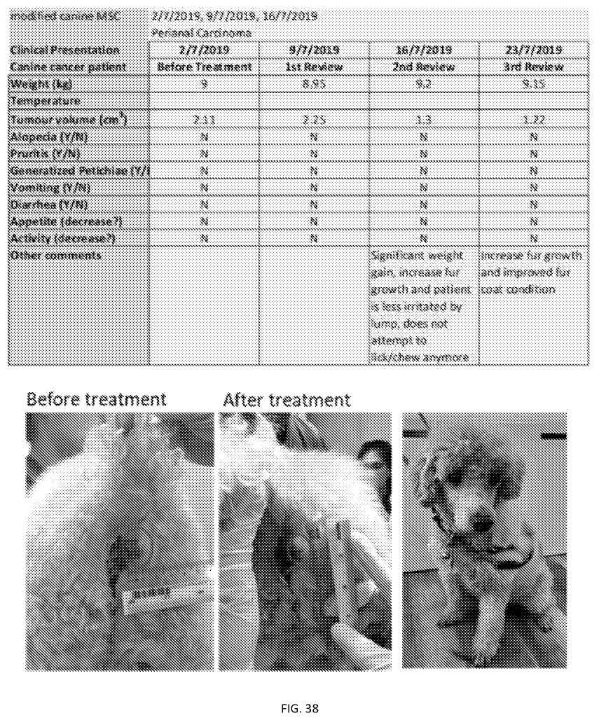

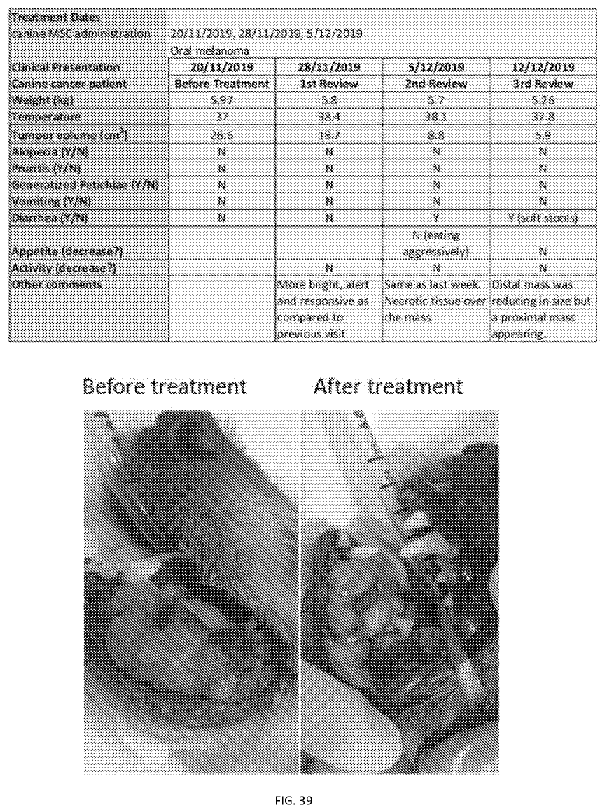

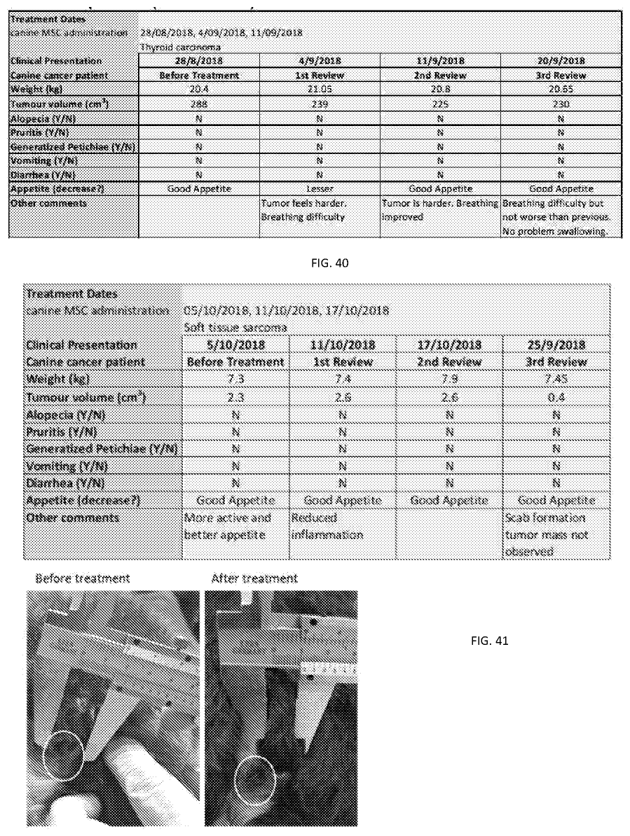

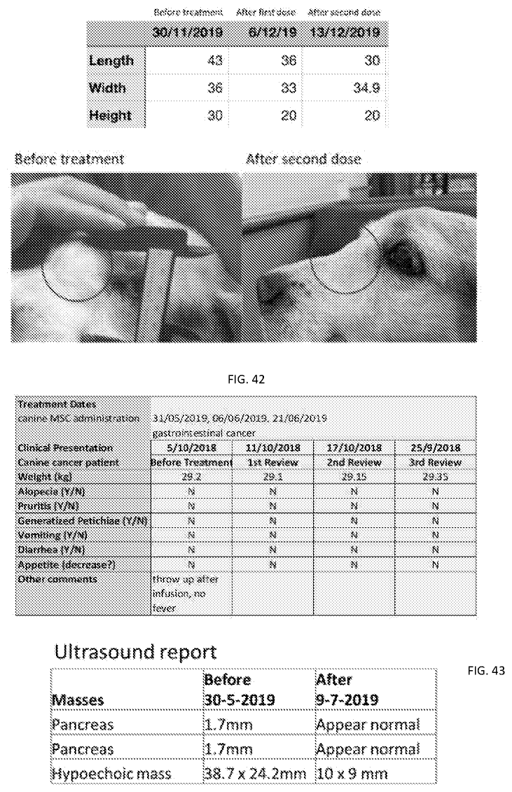

The data provided herein are representative cases from each cancer type. FIG. 38 shows perianal carcinoma data, FIG. 39 shows oral melanoma data, FIG. 40 shows thyroid carcinoma data, FIG. 41 shows soft tissue soft tissue sarcoma (cancer ulceration) data, FIG. 42 shows nasal tumor data, and FIG. 43 shows gastrointestinal cancer data.

FIG. 38 shows perianal carcinoma treatment data. Route of administration was intratumoural injection of canine CD::UPRT::GFP_MSC. Latest update (January 2020): alive, recurrence not reported. FIG. 39 shows oral melanoma treatment data. Route of administration was intratumoural injection of canine CD::UPRT::GFP_MSC. Latest update (January 2020): alive. FIG. 40 shows thyroid carcinoma treatment data. Route of administration was intratumoural injection of canine CD::UPRT::GFP_MSC. Latest update (June 2019): alive. FIG. 41 shows soft tissue sarcoma (cancer ulceration) treatment data. Route of administration was intratumoural injection of canine CD::UPRT::GFP_MSC. Latest update (November 2018): alive, no recurrence reported. Ultrasound report on 14-11-2018 showed presence of a well-defined hypoechoic round mass on the left anal area measuring 4×3×2 cm. No adhesion to the surrounding or deeper organs. No metastasis found, especially in the sublumbar lymph nodes. Few tiny 1.5 mm uroliths in the bladder, few are in the prostatic urethra. Other organs are normal. Complete Remission to date. FIG. 42 shows nasal tumour treatment data. Route of administration was intratumoral injection of canine CD::UPRT::GFP_MSC. Latest update (January 2020): alive. FIG. 43 shows gastrointestinal cancer treatment data. Route of administration was intravenous infusion of canine CD::UPRT::GFP_MSC. Latest update (July 2019): alive. From the ultrasound report despite the fact there is second growth, the original growth has decreased markedly. The details of these studies were are as set out below in the following treatment plan, with the difference for the feline patient being that modified human MSCs were used. All canine patients were treated with Cord lining MSC or adipose tissue derived MSC extracted from canine donors.

1.1 Justification of the Dose, Schedule and Route of Administration of CD::UPRT::GFP_MSC

The doses studied in the animal study ranged between 10×10⁶ to 40×10⁶ modified MSCs, and at all doses was well tolerated. For body-surface tumor, doses of 5-20×10⁶ therapeutic cells were safely administered through direct intratumoral injection. For internal masses, doses of 30×10⁶ were safely administered through direct intravenous injection. Subjects found with both body-surface and internal masses were treated with maximum 10×10⁶ (intratumoral administration) and 20×10⁶ therapeutic MSC (intravenous administration), in combination. A maximum tolerated dose was not identified in the investigation study to date.

This study uses a volume of up to 1 mL and 10 mL for intratumoral and intravenous administrations, respectively.

1.2 Justification of the Dose, Schedule, and Route of Administration of 5FC

The 5FC doses studied ranged from 20 mg/kg/day to 50 mg/kg/day, and at all doses was well tolerated. This study uses a dose of 35-50 mg/kg/day for 4-day courses of oral 5FC. This was repeated for every cycle of treatment. Information on preparation of oral 5FC was described to owner of the subject.

1.3 Study Duration and Follow-Up

Attempts were made to follow subjects who received treatment. All patients enrolled in the study were followed for survival.

1.4 Criteria for Study Termination

The Sponsor and subject's owner retains the right to terminate the study at any time.

1.5 Treatment Schedule and Follow Up Plan

The duration of the 3 cycles for each treatment is 21 days.

1.6 Treatment Guide

Since the subjects may have solid tumors at various sites (internal or surface) and sizes, investigators may choose any of the doses of modified MSCs in Table 1. When selecting the treatment, investigators should take into consideration the subject's clinical record of size and site of tumors.

TABLE
Recommended doses for various subjects, according to the site and size of tumor

| Site of | Number of | | number of | clinical |
|---|---|---|---|---|
| tumors | modified MSC | Administration | injection | record required |
| Dermal | 20 × 10\hat{ }6 | Intratumoral | up to 5 sides | size of tumor measured by |
| | | | | caliber, blood test, weight |
| Internal | 30 × 10\hat{ }6 | intravenous | NA | ultrasound, blood test, weight |
| Dermal + | 20 × 10\hat{ }6 | Intratumoral | up to 5 sides | size of tumor measured by |
| Internal | | | | caliber, blood test, weight |
| | 30 × 10\hat{ }6 | intravenous | NA | ultrasound, blood test, weight |

1.7 Procedure: Intratumoral Injection

1. Carefully swab the body surface with disinfectant for 10 seconds and air dry.

2. Surgeon to draw approximately 0.5 mL of therapeutic cells into the syringe. An Ultra-fine II short needle (30G, insulin syringe) will be used. Note: For tumor bulk containing large amount of pus, remove as much pus as possible prior to beginning injections of therapeutic cells.

3. Aspirate the contents fully from the Eppendorf tube into the syringe.

4. For body-surface tumor, therapeutic cells are administered by making multiple injections into the tumor. As much as possible, injections are made perpendicular to the tumor bulk at a depth of 0.8 cm. Inject the entire contents of the tube(s) as follows: up to 5 injections of approximately 100 μL are made (total of 0.5 mL). Inject the appropriate volume of vector slowly over ˜10 seconds and leave the needle in place for 20-25 seconds before removing. Slowly remove the needle and repeat the injection taking care to distribute the injections over the tumor bulk.

1.8 Procedure: Intravenous Administration

Intravenous administration is carried at flow rate of 10 mL in 30 min. Therapeutic cells are prepared in total volume of 5-10 mL.

1.9 Oral administration of 5FC

5FC is given orally in the form of capsules, twice a day.

On Study Procedures and Evaluations

2.2. Physical Examination

Physical examination includes auscultation of the heart and lungs, examination of the abdomen and palpation of lymph nodes. Examination of other systems should be performed if clinically indicated.

2.3. Vital Signs

Temperature and weight should be recorded at each visit.

2.4 Routine Laboratory Evaluations

CBC with differential and platelet count and chemistry panel (including electrolytes, BUN, creatinine, estimated GFR [at screening], total bilirubin, alkaline phosphatase, ALT and AST, LDH, and uric acid) are performed.

2.5. Monitoring for Potential Adverse Effect

During each review, questionnaires on sign of adverse effect should be posted to owner. For instance, subjects should be monitored for hair loss, skin rashes, nausea, vomiting diarrhea, and loss of appetite.

### Example 6—TrafEn Transfection Method is Agnostic to MSC Sources

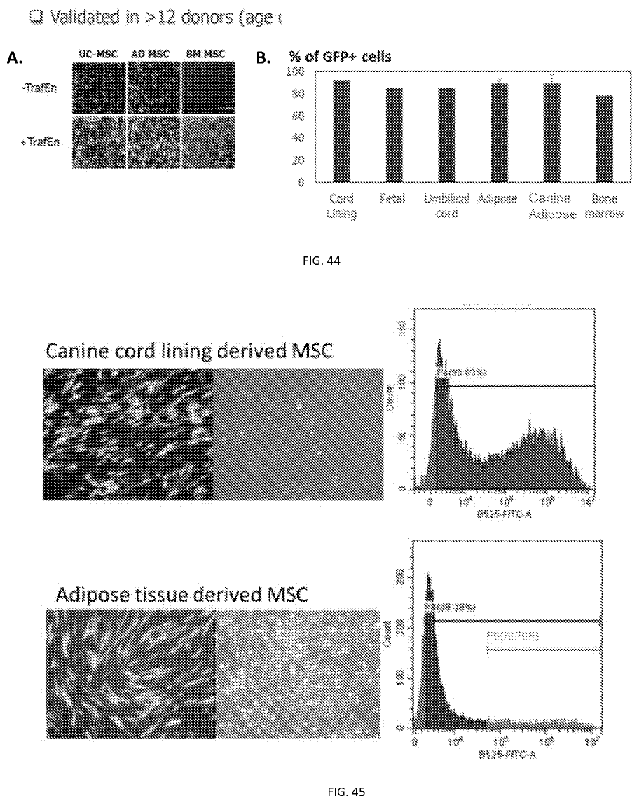

In this Example, TrafEn effect on transfection enhancement is demonstrated in the following MSC sources: Human: umbilical cord derived, Cord lining derived, adipose tissue derived, and bone marrow derived MSC. Results are shown in FIG. 44. MSC types from different commercial sources were modified with vector containing GFP transgene. Graph bar displays % of GFP+ population as measured by Flow cytometry. In FIG. 45, Canine cord lining-derived, and adipose tissue-derived MSC results are shown. MSCs from difference sources were successfully modified to express CD::UPRT::GFP.

This study was carried out using a non-centrifugation protocol as described above.

As shown in FIG. 44, the effect of TrafEn in various MSC types was determined. (A) UC-MSC (Cell Applications), AD-MSC and BM-MSC (Roosterbio) were transfected by pMAXGFP using the non-centrifugation protocol in the presence or absence of TrafEn. One day post transfection, the fluorescent images were captured. Representative images are shown. (B) MSCs obtained from cord lining (Cell Research Corp), Fetal tissue (BTI collaborator), umbilical cord (Cell Applications), Canine adipose (iVET Animal Hospital), human Adipose and bone marrow (Roosterbio) were transfected by pMAXGFP using non-centrifugation protocol in the presence of TrafEn. Two day post transfection, cells were harvested for FACS analysis. Graph presents percentage of cells expressed GFP.

FIG. 45 shows results with human cord lining (Cell Research Corp) and human adipose tissue derived (Hayandra). MSCs were transfected by CD::UPRT::GFP expression vector using the non-centrifugation protocol in the presence of TrafEn. Two day post transfection, bright field and fluorescent images were captured. After which, cells were harvested for FACS analysis. FACS profile demonstrate the different expression level in these two types.

### Example 7—Scale-Up Generation of Modified MSC Cells—Flat Bed and Microbeads Culture System

In this Example, scale-up options for generation of modified MSC cells were studied. Two approaches were developed, the first being a flat bed system and the second being a microbeads-based culture system.

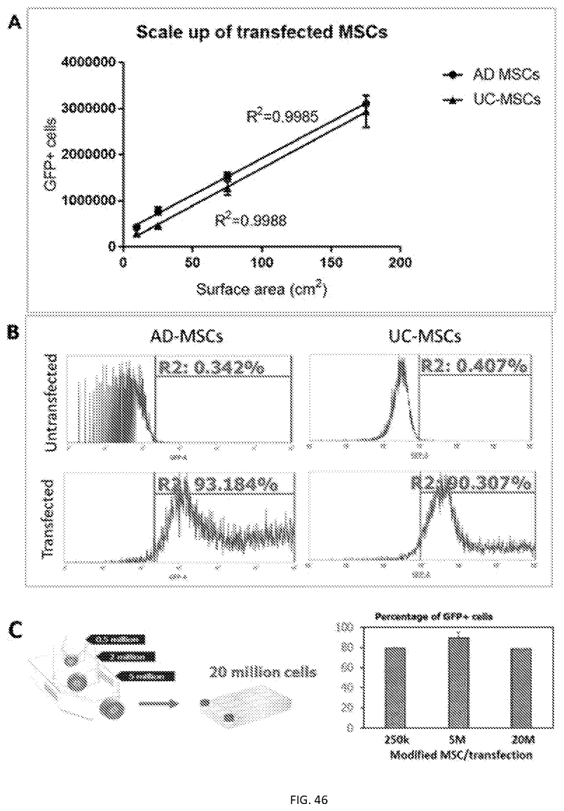

In the flat bed transfection system, a method was developed for transfection using a compatible cationic polymer and Enhancers (for example, TrafEn) to transfect MSCs efficiently. Scalability of this method in the production of modified cells by increasing the surface area of the flask was studied. It was found that the linearity of the scale up to T175 flasks (area 175 cm²) was highly correlated to the number of AD- and UC-MSCs with R2 close to 1 (FIG. 46A). According to results obtained, with a T175 flask, close to 3 million GFP+ cells can be obtained. Furthermore, the transfection efficiency remained unchanged at more than 90% as the size of the culture vessels increased (FIG. 46B). With linearity established, the size of Corning® CellSTACK® 10-stack chamber (6,360 cm²), should produce ˜1.1×10⁸ cells, sufficient for a single human patient treatment. To validate these findings, we performed transfection in a Corning® CellSTACK® one chamber (FIG. 46C). Evidently, it is feasible to upscale transfection to generate sufficient modified cells for preclinical or clinical studies, for example.

This study was carried out using a non-centrifugation protocol as described above.

FIG. 46 shows the linearity in scale up of AD-MSCs and UC-MSCs on flat-bed surfaces. AD-MSCs and UC-MSCs were transfected with CD::UPRT::GFP expression vector using non-centrifugation method in the presence of TrafEn. Without changing media, cells were harvested 2 day post transfection. (A) Number of transfected live cells were plotted against the surface area of vessel. (B) Representative images of % GFP+from FACS analysis for both AD and UC-MSCs. (C) canine cord lining MSC (Cell Research Corp) were plated in different vessel at 15000 to 20000 cells/cm2. One day later, cells were transfected by CD::UPRT::GFP polyplexes in the presence of TrafEn. After 24 h incubation, cells were harvested. Total cell number collected was determined with automated cell counter NC-3000. GFP expression was analyzed with flow cytometry. Graph presents % of GFP positive cells in various cell number collected from the culture vessels.

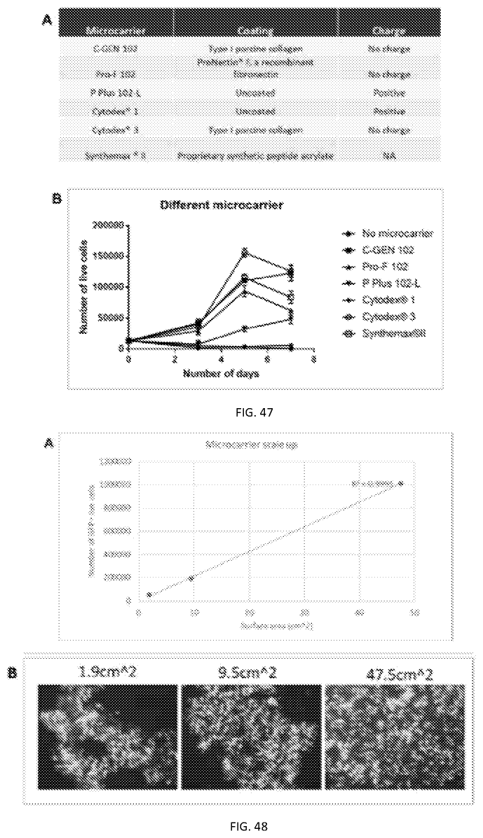

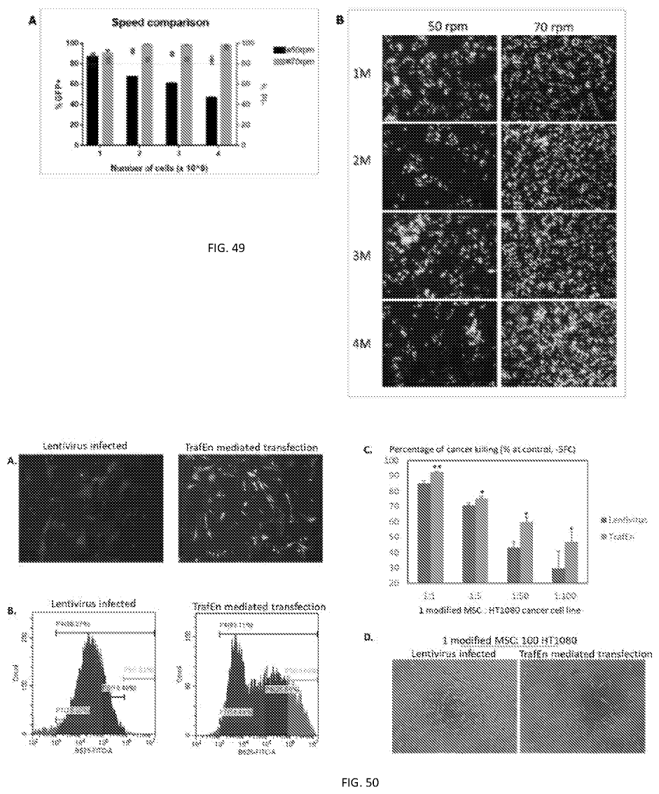

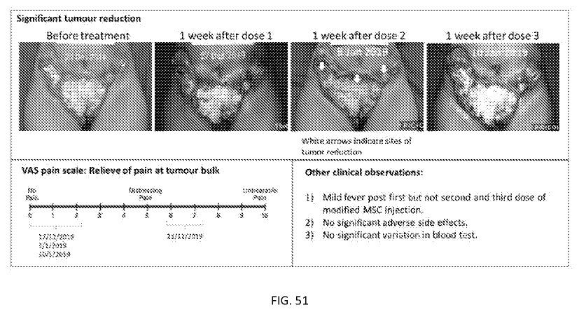

As MSCs are adherent to surfaces, growth in bioreactors/shaker flasks may utilize microcarriers for attachment. In the microcarriers-based (e.g. microbeads-based) transfection system described in this example, several microcarriers were explored to determine the compatibility for growth of MSCs. These microcarriers featured different properties such as their diameter, density, coating and charge as summarized (FIG. 47A). Studies were carried out to identify the compatibility of different microcarriers for MSC growth by measuring cell viability. Growth on uncoated microcarriers Cytodex® 1 and P PLUS 102-L yielded the least number of cells (FIG. 47B), with Cytodex®1 there was no viable MSCs, similar to the no microcarrier control sample. MSCs grew well on Type 1 porcine collagen coated microcarrier Cytodex® 3, giving rise to highest number of cells on day 5. Hence, Cytodex® 3 was selected for further studies. The number of transfected cells was linearly correlated to the total surface area of the microcarriers (FIG. 48). Interestingly, when MSCs were adapted to suspension, with increasing cell number, there was a decrease in % GFP+expression. As shaking speed of flask may affect the aggregation of cells which can physically occlude the exposure to polyplex, we increased the agitation speed of MSCs upon seeding so as to disperse the cells better. Increasing the shaking speed to 70 rpm, more cells were transfected (>90% of cells expressed GFP). Interestingly, while increasing speed decreased cell viability in conditions with lower cell numbers (1×10⁶ to 3×10⁶), possibly due to increased shear stress. This was mitigated by increasing cell number at time of seeding (4×10⁶ cells) (FIG. 49). In this study, ˜4×10⁶ GFP+ cells were obtained from 50 mL volume on ˜150 cm² microcarriers in a 125 mL Erlenmeyer flask. These results demonstrated the possibility of increasing the density of cells to ˜1×10⁸ of cells if we were to use a 4 L Erlenmeyer flask with dimensions of ˜3750 cm² in 1.25 L volume.

This study was carried out using a non-centrifugation protocol as described above, with additional shaking procedure. More detail on this protocol is described below:

Protocol for Microcarrier Culture

AD-MSC (RoosterBio), were cultured in various microcarriers, namely, C-GEN 102, Pro-F 102, P Plus 102-L (Thermofisher), Cytodex® 1, microcarrier beads, Cytodex® 3 microcarrier beads (GE Healthcare's Life Sciences) and Synthemax® II microcarriers (Corning), according to manufacturer's instruction. Briefly, microcarriers were hydrated in PBS (20 mg/mL) before sterilization using the autoclave 121° C. for 30 min. Microcarrier surface of 1.9 cm² was used for 24-well plates. Before culturing AD-MSCs, microcarriers were equilibrated in complete media for 1 h at 37° C. before use. AD-MSCs were then cultured and seeded on microcarriers, with agitation speed of 50 or 70 rpm for growth and transfection studies.

FIG. 47 describes the results with different microcarriers in AD MSCs. (A) Description of the microcarriers used (B) Number of live cells grown on different microcarrier at different days were plotted.

FIG. 48 illustrates the results of scaling from microcarriers on plates to flask. Human AD-MSCs were plated on the microcarrier according to the total surface area. Larger surface area is obtained with increasing number of microcarriers in the culture. The cells were plated on microcarriers at 20-40×10\hat{ }3 cells per cm3. One day later, cells were transfection by pMAXGFP polyplexes in the presence of TrafEn. Cells were harvested one day post transfection for cell count and FACS analysis. (A) Number of transfected live cells were plotted against the surface area of vessel. (B) Representative images of transfected cells were taken at 4× magnification.

For microcarrier transfection, AD-MSCs were seeded at different seeding densities on 1.9 cm² Cytodex® 3 microcarriers in 24-well non-adherent plates, with agitation speed of 50 rpm for 24 h before transfection. Similar to flat-bed transfection, the polymer and DNA complex were added to the cell culture using a dropwise manner after 15 min incubation. Similarly, transfection enhancers were supplemented to complete media before the addition of polyplex. For microcarrier (large-scale) transfection, AD-MSCs were seeded on Cytodex® 3 at an optimized cell density (20,000 to 40,000/cm²) of various surface area accordingly in 125 mL Erlenmeyer flasks with agitation speed of 50 or 70 rpm for transfection. Agitation speed is constant throughout the incubation and production of modified MSC.

### Example 8—MSC Modified by Non-Viral Method Demonstrates Higher Anti-Cancer Potency than MSC Modified by Lentivirus

This Example aims to compare the cancer killing efficiency of CD::UPRT::GFP_MSC generated with lentivirus and TrafEn mediated transfection. To do this, MSC stably expressing CD::UPRT::GFP was generated through antibiotic selection post lentivirus infection. The fluorescent images and flow cytometry analysis (FIG. 50A, 50B) suggest the overall expression level of transgene is significantly lower in the transduced MSC (stably express CD::UPRT::GFP). Evidently, 19% and 25.7% of TrafEn modified population expressed medium (blue) and high (orange) level of CD::UPRT::GFP, respectively (50B). In the transduced MSC population, <20% of the population expressed medium level of CD::UPRT::GFP. Thus, transduced MSC exerted lower cancer killing efficiency especially at the ratio of 1 MSC to 50 and 100 cancer cells.

FIG. 50 shows results of comparison of CD::UPRT::GFP expression and anticancer efficiency of AT-MSC modified by lentivirus or TrafEn mediated transfection method. (A) Three days post infection, MSC were subjected to 1 ug/mL puromycin selection for 2-weeks. After the establishment of MSC stably expressed CD::UPRT::GFP, another set of experiment was set up to generate CD::UPRT::GFP_MSC by TrafEn mediated transfection. Two days post transfection, fluorescent images of modified MSC were captured. (B) After which, both cultures were harvested and subjected to (B) FACS analysis and (C, D) coculture study. The graph bar represents cancer killing efficiencies at various ratios of 1 MSC to 1, 5, 50, 100 cancer cells, obtained through MTS assay. Significant differences in cancer killing efficacies of CD::UPRT::GFP_MSC generated by lentivirus versus TrafEn method, was assessed with two-tailed Student's t-test; **, p-value<0.005; *,p-value<0.05. The bright field images were taken at the end of the coculture experiment.

Methods:

Non viral MSC: as prepared using the non-centrifugation protocol as described above.

Lentivirus: MSCs were infected by lentiviral vector carrying CD::UPRT::GFP.

MSCs were transduced by lentivirus at MOI5 in the presence of 8 ug/mL polybrene. One day post infection, culture media was replaced with fresh media containing 1 ug/mL puromycin for 1 week. Cells were harvested for FACS analysis and co-culture study.

Expression profile.

Using flow cytometry analysis, the expression profile of MSC modified by CD::UPRT::GFP_lentivirus and CD::UPRT::GFP TrafEn are distinct. The three markers on the FACS profiles indicate high (orange), medium (blue), low (Currant) expression. Higher expression indicates higher payload of CD::UPRT::GFP. MSC modified with TrafEn method (non centrifugation) resulted in the generation of population with high expression of CD::UPRT::GFP (19%) but not lentivirus modified MSC. The higher payload could result in higher cancer killing efficiency.

Cancer Killing Efficiency

Significantly higher cancer killing efficiency was observed in treatment of cells with MSC modified by TrafEn method in comparison to MSC modified by lentivirus.

### Example 9—Case Study: Cat Lymphoma, Intravenous Injection of CD::UPRT::GFP Expressing Human Adipose Tissue Derived MSC

Preparation of therapeutic cells was performed as follows: human adipose tissue derived MSC) was transfected and cryo-preserved. On the day of administration, the frozen CD::UPRT::GFP_MSCs were thawed and formulated in hypothermic solution for intravenous administration. Because of an understanding of lymphoma with potential bone marrow involvement with no obvious mass to measure, it was decided that a good indication of improvement would be the PCV (Packed Cell Value). During treatment duration, the number of transfusions required for patient has reduced from 3 times per week to 0 or once per week. With the increasing value of PCV, it suggests that the anaemia is less severe. Additionally, the following observations were reported by the owner:
1) More active
2) Regaining previous habits like greeting the owner at the door and interactions with owner.
3) Improvement in appetite.

| | | transfusion/ |
|---|---|---|
| | PCV | week |
| Before treatment | 16 | 3 |
| Dose 1 | 29 | 0 |
| Dose 2 | 17 | 1 |
| Dose 3 | 35 | 1 |
| 2 weeks | 31 | 0 |
| post treatment | | |

The treatment plan for this study is as described in the canine treatment protocol outlined above. MSCs are as described above, modified using the non centrifugation method in the presence of TrafEn

### Example 10—Compassionate Use: Intratumoral Injection of CD::UPRT::GFP Expressing MSC in Recurrent Clear Cell Carcinoma, 46 Year Old Patient

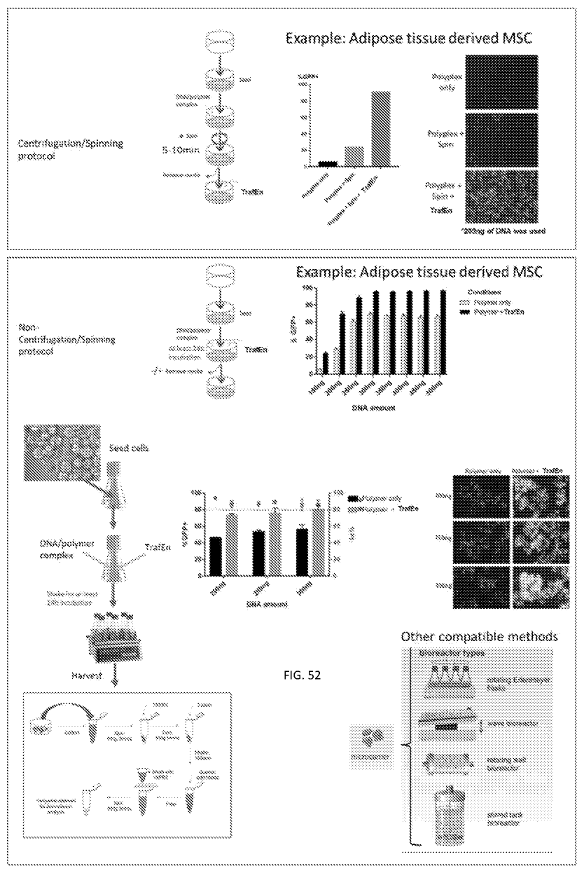

A compassionate use treatment was performed on a 46 year old patient having recurrent clear cell carcinoma. The subject was treated by intratumoral injection of CD::UPRT::GFP expressing MSCs as described herein. Results are shown in FIG. 51.

Adipose tissue MSC was extracted from the patient's tissue obtained through liposuction. After expansion of MSCs, a cell bank is created in Hayandra Peduli's GMP facility. Transfection protocol was optimized. In the presence of TrafEn, 6M MSCs were transfected in T225 flask and transfection efficiency up to 75% can be achieved. TrafEn protocol and reagents are transferred to the GMP facility for generation of CD::UPRT::GFP_MSC for compassionate use.

The modified MSCs were harvested on the day of treatment and prepared in 2 mL of plasmalyte. 20 to 50M cells were injected into 20 sites intratumorally at the peripheral of the tumor bulk. One day post MSC administration, 5FC were given to patient through oral administration at total of 2000 mg 5FC/day (4×500 mg 5FC pill per day). 5FC were administrated for 4 days.

The cycle of MSC and 5FC administration repeated every week. Data shown the pictures of the tumor bulk and pain level 1 week after each treatment cycle.

### Example 11—Centrifugation Versus Non-Centrifugation Methods

FIG. 52 provides a schematic depiction of a typical Centrifugation/Spinning-based transfection method (top), as compared with examples of non-centrifugation/spinning transfection methods (bottom).

In the centrifugation/spinning-based transfection, cells are seeded to the vessel, and DNA/polymer complex is added. In the depicted example, a spinning step is performed as indicated for about 5-10 minutes. The media is then removed, and enhancer (i.e. TrafEn) is added. Also provided is data collected from adipose tissue derived MSC treated in such a manner.

For comparison, a non-centrifugation/spinning transfection method is shown in the bottom panel of FIG. 52. Multiple options are depicted for this approach. In one embodiment, cells are seeded to the vessel, DNA/polymer complex is added and enhancer (e.g. TrafEn) is also added, and then incubation is performed for at least about 24 hours (no spinning/centrifugation is performed here). The media is then removed, providing the transfected MSCs. Data is shown for Adipose tissue-derived MSCs subject to such treatment.

Another embodiment of the non-centrifugation/spinning transfection method is shown in FIG. 52 (bottom). In this embodiment, cells are seeded (top graph/image—on flatbed; bottom image—on microbeads, and DNA/polymer complex as well as enhancer (e.g. TrafEn) is added. Incubation is performed for at least about 24 hours, while shaking (other options are also depicted, such as bioreactor-types including rotating flasks, wave bioreactor systems, rotating wall bioreactor designs, and stirred tank bioreactor designs, for example). Harvesting is then performed by collecting, followed by a spin (for example, about 300 g for 3 mins in the depicted example), addition of 1×PBS, followed by another spin (for example, about 300 g for 3 mins in the depicted example), followed by addition of trypsin, shaking at 100 rpm, and quenching with media. Filtration is performed, along with washing (with 1×PBS in the depicted example). Spinning is performed (for example, about 300 g for 3 mins in the depicted example), and a cell pellet is thus obtained for downstream analysis. Data is shown for cells treated in such manner, and a comparison of polymer only versus polymer+TrafEn is provided.

### Example 12—Cryopreserved Modified MSCs (Generated Using TrafEn) are Viable and Functional

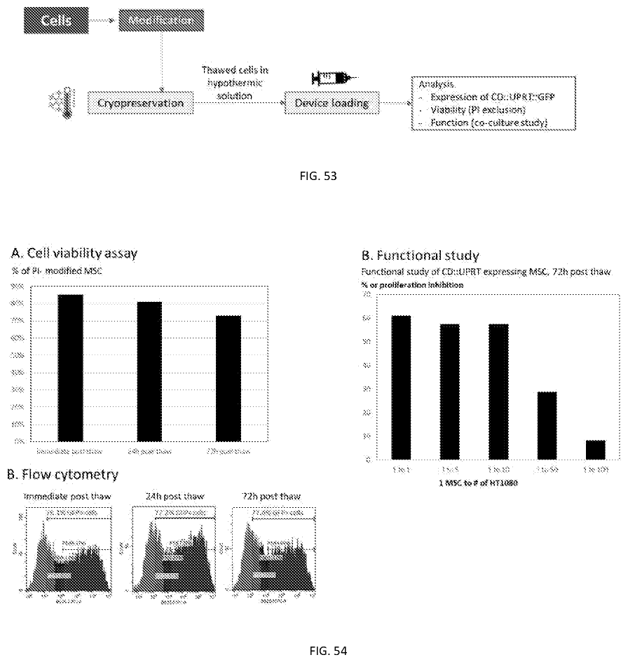

FIG. 53 provides a schematic depiction of a workflow for cryopreserving modified MSCs (prepared using TrafEn) so as to allow for long term storage thereof. As shown, modified MSCs may be placed in cryopreservation storage. When needed, the cells may be removed from storage and prepared for use by thawing in a hypothermic solution.

FIG. 54 shows results for cell viability, expression level, and functional activity of modified MSCs that were cryopreserved and then thawed as shown in FIG. 53 described above. As shown, the modified MSCs retained high cell viability and expression level after cryopreservation thawing and preservation in hypothermic solution up to 72 h.

The cells were thawed in hypothermic solution (Hypothermosol) and stored at 4 C for up to 3 days. The cell viability and % of CD::UPRT::GFP+ cells were measured every day.

One or more illustrative embodiments have been described by way of example. It will be understood to persons skilled in the art that a number of variations and modifications can be made without departing from the scope of the invention as defined in the claims.

### REFERENCES

1. Sinden, J. D., et al., Human Neural Stem Cell Therapy for Chronic Ischemic Stroke: Charting Progress from Laboratory to Patients. Stem Cells and Development, 2017. 26(13): p. 933-947.
2. Lalu, M. M., et al., Safety of cell therapy with mesenchymal stromal cells (SafeCell): a systematic review and meta-analysis of clinical trials. PLoS One, 2012. 7(10): p. e47559.
3. Liu, S., et al., Strategies to Optimize Adult Stem Cell Therapy for Tissue Regeneration. Int J Mol Sci, 2016. 17(6).
4. Phillips, M. I. and Y. L. Tang, Genetic modification of stem cells for transplantation. Adv Drug Deliv Rev, 2008. 60(2): p. 160-72.
5. Phillips, M. I. and Y. Tang, Genetic modification of stem cells for cardiac, diabetic, and hemophilia transplantation therapies. Prog Mol Biol Transl Sci, 2012. 111: p. 285-304.
6. The Cell and Gene Therapy Catapult UK clinical trials database. 2016.
7. Zhang, J., V. Kale, and M. Chen, Gene-directed enzyme prodrug therapy. AAPS J, 2015. 17(1): p. 102-10.
8. Altaner, C., Prodrug cancer gene therapy. Cancer Letters. 270(2): p. 191-201.
9. Li, Z., D. Fan, and D. Xiong, Mesenchymal stem cells as delivery vectors for anti-tumor therapy. Stem Cell Investig, 2015. 2: p. 6.
10. Marofi, F., et al., Mesenchymal Stromal/Stem Cells: A New Era in the Cell-Based Targeted Gene Therapy of Cancer. Frontiers in Immunology, 2017. 8: p. 1770.
11. Brown, N. L. and N. R. Lemoine, Clinical trials with GDEPT: cytosine deaminase and 5-fluorocytosine. Methods Mol Med, 2004. 90: p. 451-7.
12. Niu, Y., J. S. Li, and X. R. Luo, Enhancement of expression of survivin promoter-driven CD/TK double suicide genes by the nuclear matrix attachment region in transgenic gastric cancer cells. Gene, 2014. 534(2): p. 177-82.
13. Luo, X. R., et al., Adenovirus-mediated double suicide gene selectively kills gastric cancer cells. Asian Pac J Cancer Prev, 2012. 13(3): p. 781-4.
14. Zhou, G. J., et al., [In vitro killing effect of adenovirus-mediated fusion gene system driven by KDR promoter on gastric cancer cells]. Nan Fang Yi Ke Da Xue Xue Bao, 2006. 26(4): p. 402-5.
15. Qiao, L., et al., Dkk-1 secreted by mesenchymal stem cells inhibits growth of breast cancer cells via depression of Wnt signalling. Cancer Lett, 2008. 269(1): p. 67-77.
16. Matuskova, M., et al., Combined enzyme/prodrug treatment by genetically engineered AT-MSC exerts synergy and inhibits growth of MDA-MB-231 induced lung metastases. J Exp Clin Cancer Res, 2015. 34: p. 33.
17. Jung, J. H., et al., Three-dimensional assessment of bystander effects of mesenchymal stem cells carrying a cytosine deaminase gene on glioma cells. American Journal of Cancer Research, 2015. 5(9): p. 2686-2696.
18. Chang, D. Y., et al., The growth of brain tumors can be suppressed by multiple transplantation of mesenchymal stem cells expressing cytosine deaminase. Int J Cancer, 2010. 127(8): p. 1975-83.
19. Altaner, C., et al., Complete regression of glioblastoma by mesenchymal stem cells mediated prodrug gene therapy simulating clinical therapeutic scenario. Int J Cancer, 2014. 134(6): p. 1458-65.
20. Karjoo, Z., X. Chen, and A. Hatefi, Progress and problems with the use of suicide genes for targeted cancer therapy. Adv Drug Deliv Rev, 2016. 99(Pt A): p. 113-28.
21. Huber, B.E., C.A. Richards, and E.A. Austin, VDEPT: An enzyme/prodrug gene therapy approach for the treatment of metastatic colorectal cancer. Advanced Drug Delivery Reviews, 1995. 17(3): p. 279-292.
22. Li, Z., et al., Enzyme/prodrug gene therapy approach for breast cancer using a recombinant adenovirus expressing Escherichia coli cytosine deaminase. Cancer Gene Ther, 1997. 4(2): p. 113-7.
23. Kaliberov, S. A., et al., Combination of cytosine deaminase suicide gene expression with DRS antibody treatment increases cancer cell cytotoxicity. Cancer Gene Ther, 2006. 13(2): p. 203-14.
24. Koyama, F., et al., Combined suicide gene therapy for human colon cancer cells using adenovirus-mediated transfer of Escherichia coli cytosine deaminase gene and Escherichia coli uracil phosphoribosyltransferase gene with 5-fluorocytosine. Cancer Gene Ther, 2000. 7(7): p. 1015-22.
25. Shi, D. Z., et al., Pharmacokinetics and the bystander effect in CD::UPRT/5-FC bi-gene therapy of glioma. Chin Med J (Engl), 2009. 122(11): p. 1267-72.
26. Xing, L., et al., Expression of the bifunctional suicide gene CDUPRT increases radiosensitization and bystander effect of 5-FC in prostate cancer cells. Radiother Oncol, 2009. 92(3): p. 345-52.
27. Gopinath, P. and S.S. Ghosh, Implication of functional activity for determining therapeutic efficacy of suicide genes in vitro. Biotechnol Lett, 2008. 30(11): p. 1913-21.
28. Niess, H., et al., Treatment of advanced gastrointestinal tumors with genetically modified autologous mesenchymal stromal cells (TREAT-ME1): study protocol of a phase I/II clinical trial. BMC Cancer, 2015. 15(1): p. 237.
29. Aboody, K. S., et al., Neural stem cell-mediated enzyme/prodrug therapy for glioma: preclinical studies. Sci Transl Med, 2013. 5(184): p. 184ra59.
30. Tate, C. C., et al., Human mesenchymal stromal cells and their derivative, SB623 cells, rescue neural cells via trophic support following in vitro ischemia. Cell Transplant, 2010. 19(8): p. 973-84.
31. Pollock, K., et al., Human Mesenchymal Stem Cells Genetically Engineered to Overexpress Brain-derived Neurotrophic Factor Improve Outcomes in Huntington's Disease Mouse Models. Mol Ther, 2016. 24(5): p. 965-77.
32. Kaestner, L., A. Scholz, and P. Lipp, Conceptual and technical aspects of transfection and gene delivery. Bioorganic & Medicinal Chemistry Letters, 2015. 25(6): p. 1171-1176.
33. Charrier, S., et al., Quantification of lentiviral vector copy numbers in individual hematopoietic colony-forming cells shows vector dose-dependent effects on the frequency and level of transduction. Gene Therapy, 2011. 18(5): p. 479-487.
34. Cooper, M. J., et al., Safety-modified episomal vectors for human gene therapy. Proceedings of the National Academy of Sciences of the United States of America, 1997. 94(12): p. 6450-6455.
35. Batard, P., M. Jordan, and F. Wurm, Transfer of high copy number plasmid into mammalian cells by calcium phosphate transfection. Gene, 2001. 270(1-2): p. 61-8.
36. Cohen, R. N., et al., Quantification of Plasmid DNA Copies in the Nucleus after Lipoplex and Polyplex Transfection. Journal of controlled release: official journal of the Controlled Release Society, 2009. 135(2): p. 166-174.
37. van der Loo, J. C. M., et al., Scale-up and manufacturing of clinical-grade self-inactivating γ-retroviral vectors by transient transfection. Gene therapy, 2012. 19(3): p. 246-254.
38. Vicente, T., et al., Virus production for clinical gene therapy. Methods Mol Biol, 2009. 542: p. 447-70.
39. van der Loo, J. C. and J. F. Wright, Progress and challenges in viral vector manufacturing. Hum Mol Genet, 2016. 25(R1): p. R42-52.
40. Kallifatidis, G., et al., Improved lentiviral transduction of human mesenchymal stem cells for therapeutic intervention in pancreatic cancer. Cancer Gene Ther, 2008. 15(4): p. 231-40.
41. Kyriakou, C., et al., Factors that influence short-term homing of human bone marrow-derived mesenchymal stem cells in a xenogeneic animal model. Haematologica, 2008. 93(10): p. 1457-65.
42. Gwendal, L. and Y. L. Paula, Recent discoveries concerning the tumor—mesenchymal stem cell interactions. Biochim Biophys Acta, 2016. 1866(2): p. 290-299.
43. Ramamoorth, M. and A. Narvekar, Non viral vectors in gene therapy—an overview. J Clin Diagn Res, 2015. 9(1): p. GE01-6.
44. Baek, K., et al., Gene Transfection for Stem Cell Therapy. Current Stem Cell Reports, 2016. 2(1): p. 52-61.
45. Haleem-Smith, H., et al., Optimization of high-efficiency transfection of adult human mesenchymal stem cells in vitro. Mol Biotechnol, 2005. 30(1): p. 9-20.
46. Halim, N. S. S. A., et al., A Comparative Study of Non-Viral Gene Delivery Techniques to Human Adipose-Derived Mesenchymal Stem Cell. International Journal of Molecular Sciences, 2014. 15(9): p. 15044-15060.
47. Kucerova, L., et al., Cytosine deaminase expressing human mesenchymal stem cells mediated tumour regression in melanoma bearing mice. J Gene Med, 2008. 10(10): p. 1071-82.
48. Kucerova, L., et al., Adipose tissue-derived human mesenchymal stem cells mediated prodrug cancer gene therapy. Cancer Res, 2007. 67(13): p. 6304-13.
49. Ho, Y. K., et al., Enhanced non-viral gene delivery by coordinated endosomal release and inhibition of beta-tubulin deactylase. Nucleic Acids Res, 2017. 45(6): p. e38.
50. Zou, R., et al., Combinatorial engineering of 1-deoxy-D-xylulose 5-phosphate pathway using cross-lapping in vitro assembly (CLIVA) method. PLoS One, 2013. 8(11): p. e79557.
51. Dominici, M., et al., Minimal criteria for defining multipotent mesenchymal stromal cells. The International Society for Cellular Therapy position statement. Cytotherapy, 2006. 8(4): p. 315-7.
52. Han, I., et al., Differentiation Potential of Mesenchymal Stem Cells Is Related to Their Intrinsic Mechanical Properties. Int Neurourol J, 2017. 21(Suppl 1): p. 94-31.
53. Fernandez, T.d.S.F.a.C.d.S., Mesenchymal Stem Cells: Biological Characteristics and Potential Clinical Applications for Haematopoietic Stem Cell Transplantation, Pluripotent Stem Cells—From the Bench to the Clinic Dr. Minoru Tomizawa (Ed.), in InTech2016.
54. Ho, Y. K., et al., Enhanced non-viral gene delivery by coordinated endosomal release and inhibition of beta-tubulin deactylase. Nucleic Acids Res, 2016.
55. Bourbeau, D., et al., Suicide gene therapy with an adenovirus expressing the fusion gene CD::UPRT in human glioblastomas: different sensitivities correlate with p53 status. J Gene Med, 2004. 6(12): p. 1320-32.
56. Pulukuri, S. M. K., et al., Epigenetic Upregulation of Urokinase Plasminogen Activator Promotes the Tropism of Mesenchymal Stem Cells for Tumor Cells. Molecular cancer research: MCR, 2010. 8(8): p. 1074-1083.
57. Kim, E.-K., et al., Endogenous Gastric-Resident Mesenchymal Stem Cells Contribute to Formation of Cancer Stroma and Progression of Gastric Cancer. Korean Journal of Pathology, 2013. 47(6): p. 507-518.
58. Kim, S. M., et al., In vivo near-infrared imaging for the tracking of systemically delivered mesenchymal stem cells: tropism for brain tumors and biodistribution. Int J Nanomedicine, 2016. 11: p. 13-23.
59. Xie, C., et al., Systemically Infused Mesenchymal Stem Cells Show Different Homing Profiles in Healthy and Tumor Mouse Models. Stem Cells Transl Med, 2017. 6(4): p. 1120-1131.
60. Cavarretta, I. T., et al., Adipose tissue-derived mesenchymal stem cells expressing prodrug-converting enzyme inhibit human prostate tumor growth. Mol Ther, 2010. 18(1): p. 223-31.
61. Wongrakpanich, A., et al., The absence of CpG in plasmid DNA-chitosan polyplexes enhances transfection efficiencies and reduces inflammatory responses in murine lungs. Mol Pharm, 2014. 11(3): p. 1022-31.
62. Moritz, B., P. B. Becker, and U. Gopfert, CMV promoter mutants with a reduced propensity to productivity loss in CHO cells. Sci Rep, 2015. 5: p. 16952.
63. Zhao, C. P., et al., Matrix attachment region combinations increase transgene expression in transfected Chinese hamster ovary cells. Sci Rep, 2017. 7: p. 42805.
64. Altanerova, V., et al., Human adipose tissue-derived mesenchymal stem cells expressing yeast cytosinedeaminase::uracil phosphoribosyltransferase inhibit intracerebral rat glioblastoma. Int J Cancer, 2012. 130(10): p. 2455-63.
65. Meyerrose, T. E., et al., Lentiviral-transduced human mesenchymal stem cells persistently express therapeutic levels of enzyme in a xenotransplantation model of human disease. Stem Cells, 2008. 26(7): p. 1713-22.
66. Liu, Y., et al., Lentiviral-mediated gene transfer into human adipose-derived stem cells: role of NELL1 versus BMP2 in osteogenesis and adipogenesis in vitro. Acta Biochim Biophys Sin (Shanghai), 2012. 44(10): p. 856-65.
67. Lin, P., et al., Polybrene inhibits human mesenchymal stem cell proliferation during lentiviral transduction. PLoS One, 2011. 6(8): p. e23891.
68. Khatri, A., et al., Combination of cytosine deaminase with uracil phosphoribosyl transferase leads to local and distant bystander effects against RM1 prostate cancer in mice. J Gene Med, 2006. 8(9): p. 1086-96.
69. Abrate, A., et al., Mesenchymal stem cells expressing therapeutic genes induce autochthonous prostate tumour regression. Eur J Cancer, 2014. 50(14): p. 2478-88.
70. Portnow, J., et al., Neural Stem Cell-Based Anticancer Gene Therapy: A First-in-Human Study in Recurrent High-Grade Glioma Patients. Clin Cancer Res, 2017. 23(12): p. 2951-2960.
71. Rader, R. Cell and Gene Therapies: Industry Faces Potential Capacity Shortages. 2017. 37.
72. Niess, H., et al., Treatment of advanced gastrointestinal tumors with genetically modified autologous mesenchymal stromal cells (TREAT-ME1): study protocol of a phase I/II clinical trial. BMC Cancer, 2015. 15(1): p. 237.
73. Aboody, K. S., et al., Neural stem cell-mediated enzyme/prodrug therapy for glioma: preclinical studies. Sci Transl Med, 2013. 5(184): p. 184ra59.
74. Tate, C. C., et al., Human mesenchymal stromal cells and their derivative, SB623 cells, rescue neural cells via trophic support following in vitro ischemia. Cell Transplant, 2010. 19(8): p. 973-84.
75. Pollock, K., et al., Human Mesenchymal Stem Cells Genetically Engineered to Overexpress Brain-derived Neurotrophic Factor Improve Outcomes in Huntington's Disease Mouse Models. Mol Ther, 2016. 24(5): p. 965-77.
76. Kaestner, L., A. Scholz, and P. Lipp, Conceptual and technical aspects of transfection and gene delivery. Bioorganic & Medicinal Chemistry Letters, 2015. 25(6): p. 1171-1176.
77. Charrier, S., et al., Quantification of lentiviral vector copy numbers in individual hematopoietic colony-forming cells shows vector dose-dependent effects on the frequency and level of transduction. Gene Therapy, 2011. 18(5): p. 479-487.
78. Cooper, M. J., et al., Safety-modified episomal vectors for human gene therapy. Proceedings of the National Academy of Sciences of the United States of America, 1997. 94(12): p. 6450-6455.
79. Batard, P., M. Jordan, and F. Wurm, Transfer of high copy number plasmid into mammalian cells by calcium phosphate transfection. Gene, 2001. 270(1-2): p. 61-8.
80. Cohen, R. N., et al., Quantification of Plasmid DNA Copies in the Nucleus after Lipoplex and Polyplex Transfection. Journal of controlled release: official journal of the Controlled Release Society, 2009. 135(2): p. 166-174.
81. van der Loo, J. C. M., et al., Scale-up and manufacturing of clinical-grade self-inactivating y-retroviral vectors by transient transfection. Gene therapy, 2012. 19(3): p. 246-254.
82. Vicente, T., et al., Virus production for clinical gene therapy. Methods Mol Biol, 2009. 542: p. 447-70.
83. van der Loo, J. C. and J. F. Wright, Progress and challenges in viral vector manufacturing. Hum Mol Genet, 2016. 25(R1): p. R42-52.
84. Kolata, G., Gene Therapy Hits a Peculiar Roadblock: A Virus Shortage. 2017, The New York Times.
85. Stanton, D. ‘Dramatic’ Gene Therapy Demand Drives Viral Vector Delays. 2018.
86. Mammoser, G. Virus Shortage Impacting Gene Therapy Experiments. 2017.
87. Challener, C.A. Cell and Gene Therapies Face Manufacturing Challenges. 2017.
88. Kallifatidis, G., et al., Improved lentiviral transduction of human mesenchymal stem cells for therapeutic intervention in pancreatic cancer. Cancer Gene Ther, 2008. 15(4): p. 231-40.
89. Kyriakou, C., et al., Factors that influence short-term homing of human bone marrow-derived mesenchymal stem cells in a xenogeneic animal model. Haematologica, 2008. 93(10): p. 1457-65.
90. Gwendal, L. and Y. L. Paula, Recent discoveries concerning the tumor—mesenchymal stem cell interactions. Biochim Biophys Acta, 2016. 1866(2): p. 290-299.
91. Ramamoorth, M. and A. Narvekar, Non viral vectors in gene therapy- an overview. J Clin Diagn Res, 2015. 9(1): p. GE01-6.
92. Baek, K., et al., Gene Transfection for Stem Cell Therapy. Current Stem Cell Reports, 2016. 2(1): p. 52-61.
93. Haleem-Smith, H., et al., Optimization of high-efficiency transfection of adult human mesenchymal stem cells in vitro. Mol Biotechnol, 2005. 30(1): p. 9-20.
94. Kim, S. M., et al., In vivo near-infrared imaging for the tracking of systemically delivered mesenchymal stem cells: tropism for brain tumors and biodistribution. Int J Nanomedicine, 2016. 11: p. 13-23.
95. Xie, C., et al., Systemically Infused Mesenchymal Stem Cells Show Different Homing Profiles in Healthy and Tumor Mouse Models. Stem Cells Transl Med, 2017. 6(4): p. 1120-1131.
96. Cavarretta, I. T., et al., Adipose tissue-derived mesenchymal stem cells expressing prodrug-converting enzyme inhibit human prostate tumor growth. Mol Ther, 2010. 18(1): p. 223-31.
97. Altaner, C., et al., Complete regression of glioblastoma by mesenchymal stem cells mediated prodrug gene therapy simulating clinical therapeutic scenario. Int J Cancer, 2014. 134(6): p. 1458-65.
98. Wongrakpanich, A., et al., The absence of CpG in plasmid DNA-chitosan polyplexes enhances transfection efficiencies and reduces inflammatory responses in murine lungs. Mol Pharm, 2014. 11(3): p. 1022-31.
99. Moritz, B., P. B. Becker, and U. Gopfert, CMV promoter mutants with a reduced propensity to productivity loss in CHO cells. Sci Rep, 2015. 5: p. 16952.
100. Zhao, C. P., et al., Matrix attachment region combinations increase transgene expression in transfected Chinese hamster ovary cells. Sci Rep, 2017. 7: p. 42805.
101. Jackson, D.A., S. Juranek, and H.J. Lipps, Designing Nonviral Vectors for Efficient Gene Transfer and Long-Term Gene Expression. Molecular Therapy, 2006. 14(5): p. 613-626.
102. Tu, G., et al., Non-replicating Epstein-Barr virus-based plasmids extend gene expression and can improve gene therapy in vivo. J Biol Chem, 2000. 275(39): p. 30408-16.
103. Verghese, S.C., et al., S/MAR sequence confers long-term mitotic stability on non-integrating lentiviral vector episomes without selection. Nucleic acids research, 2014. 42(7): p. e53-e53.
104. Dominici, M., et al., Minimal criteria for defining multipotent mesenchymal stromal cells. The International Society for Cellular Therapy position statement. Cytotherapy, 2006. 8(4): p. 315-7.
105. Oncotarget. 2017 Oct. 6; 8(46): 80156-80166
106. Clin Cancer Res. 2017 Jun. 15; 23(12):2951-2960
107. Molecular Therapy, 2006. 14(5): p. 613-626
108. J Biol Chem, 2000. 275(39): p. 30408-16
109. Nucleic acids research, 2014. 42(7): p. e53-e53
110. WO2014/070111 entitled A Novel Reagent for Gene-Drug Therapeutics
111. Ho Y. K., et al., Enhanced Non-Viral Gene Delivery by Coordinated Endosomal Release and Inhibition of β-Tubulin Deactylase, Nucleic Acids Research, 2017, 45(6): e38.
112. Lalu, M. M. et al. Safety of cell therapy with mesenchymal stromal cells (SafeCell): a systematic review and meta-analysis of clinical trials. PLoS One 7, e47559, doi:10.1371/journal.pone.0047559 (2012).
113. Liu, S. et al. Strategies to Optimize Adult Stem Cell Therapy for Tissue Regeneration. Int J Mol Sci 17, doi:10.3390/ijms17060982 (2016).
114. Phillips, M. I. & Tang, Y. L. Genetic modification of stem cells for transplantation. Adv Drug Deliv Rev 60, 160-172, doi:10.1016/j.addr.2007.08.035 (2008).
115. Noronha Nc, N. C. et al. Priming approaches to improve the efficacy of mesenchymal stromal cell-based therapies. Stem Cell Res Ther 10, 131, doi:10.1186/s13287-019-1224-y (2019).
116. Galipeau, J. & Sensebe, L. Mesenchymal Stromal Cells: Clinical Challenges and Therapeutic Opportunities. Cell Stem Cell 22, 824-833, doi:10.1016/j.stem.2018.05.004 (2018).
117. Mohr, A. & Zwacka, R. The future of mesenchymal stem cell-based therapeutic approaches for cancer—From cells to ghosts. Cancer letters 414, 239-249, doi:10.1016/j.canlet.2017.11.025 (2018).
118. Moradian Tehrani, R. et al. Mesenchymal stem cells: A new platform for targeting suicide genes in cancer. Journal of cellular physiology 233, 3831-3845, doi:10.1002/jcp.26094 (2018).
119. Zhang, J., Kale, V. & Chen, M. Gene-directed enzyme prodrug therapy. AAPS J 17, 102-110, doi:10.1208/s12248-014-9675-7 (2015).
120. Altaner, C. Prodrug cancer gene therapy. Cancer letters 270, 191-201, doi:10.1016/j.canlet.2008.04.023.
121. Niu, Y., Li, J. S. & Luo, X. R. Enhancement of expression of survivin promoter-driven CD/TK double suicide genes by the nuclear matrix attachment region in transgenic gastric cancer cells. Gene 534, 177-182, doi:10.1016/j.gene.2013.10.064 (2014).
122. You, M. H. et al. Cytosine deaminase-producing human mesenchymal stem cells mediate an antitumor effect in a mouse xenograft model. Journal of gastroenterology and hepatology 24, 1393-1400, doi:10.1111/j.1440-1746.2009.05862.x (2009).
123. Qiao, L., Xu, Z. L., Zhao, T. J., Ye, L. H. & Zhang, X. D. Dkk-1 secreted by mesenchymal stem cells inhibits growth of breast cancer cells via depression of Wnt signalling. Cancer letters 269, 67-77, doi:10.1016/j.canlet.2008.04.032 (2008).
124. Matuskova, M. et al. Combined enzyme/prodrug treatment by genetically engineered AT-MSC exerts synergy and inhibits growth of MDA-MB-231 induced lung metastases. J Exp Clin Cancer Res 34, 33, doi:10.1186/s13046-015-0149-2 (2015).
125. Jung, J. H. et al. Three-dimensional assessment of bystander effects of mesenchymal stem cells carrying a cytosine deaminase gene on glioma cells. American Journal of Cancer Research 5, 2686-2696 (2015).
126. Altaner, C. et al. Complete regression of glioblastoma by mesenchymal stem cells mediated prodrug gene therapy simulating clinical therapeutic scenario. Int J Cancer 134, 1458-1465, doi:10.1002/ijc.28455 (2014).
127. Karjoo, Z., Chen, X. & Hatefi, A. Progress and problems with the use of suicide genes for targeted cancer therapy. Adv Drug Deliv Rev 99, 113-128, doi:10.1016/j.addr.2015.05.009 (2016).
128. Shi, D. Z. et al. Pharmacokinetics and the bystander effect in CD::UPRT/5-FC bi-gene therapy of glioma. Chin Med J (Engl) 122, 1267-1272 (2009).
129. Xing, L. et al. Expression of the bifunctional suicide gene CDUPRT increases radiosensitization and bystander effect of 5-FC in prostate cancer cells. Radiother Oncol 92, 345-352, doi:10.1016/j.radonc.2009.04.003 (2009).
130. Gopinath, P. & Ghosh, S. S. Implication of functional activity for determining therapeutic efficacy of suicide genes in vitro. Biotechnol Lett 30, 1913-1921, doi:10.1007/s10529-008-9787-1 (2008).
131. Niess, H. et al. Treatment of advanced gastrointestinal tumors with genetically modified autologous mesenchymal stromal cells (TREAT-ME1): study protocol of a phase I/II clinical trial. BMC Cancer 15, 237, doi:10.1186/s12885-015-1241-x (2015).
132. Aboody, K. S. et al. Neural stem cell-mediated enzyme/prodrug therapy for glioma: preclinical studies. Sci Transl Med 5, 184ra159, doi:10.1126/scitranslmed.3005365 (2013).
133. Pollock, K. et al. Human Mesenchymal Stem Cells Genetically Engineered to Overexpress Brain-derived Neurotrophic Factor Improve Outcomes in Huntington's Disease Mouse Models. Mol Ther 24, 965-977, doi:10.1038/mt.2016.12 (2016).
134. Ciuffi, A. The benefits of integration. Clinical Microbiology and Infection 22, 324-332, doi:https://doi.org/10.1016/j.cmi.2016.02.013 (2016).
135. Thomas, C. E., Ehrhardt, A. & Kay, M. A. Progress and problems with the use of viral vectors for gene therapy. Nat Rev Genet 4, 346-358, doi:10.1038/nrg1066 (2003).
136. Dewey, R. A. et al. Chronic brain inflammation and persistent herpes simplex virus 1 thymidine kinase expression in survivors of syngeneic glioma treated by adenovirus-mediated gene therapy: implications for clinical trials. Nat Med 5, 1256-1263, doi:10.1038/15207 (1999).
137. Vicente, T., Peixoto, C., Carrondo, M. J. & Alves, P. M. Virus production for clinical gene therapy. Methods Mol Biol 542, 447-470, doi:10.1007/978-1-59745-561-9_24 (2009).
138. van der Loo, J. C. & Wright, J. F. Progress and challenges in viral vector manufacturing. Hum Mol Genet 25, R42-52, doi:10.1093/hmg/ddy451 (2016).
139. Charrier, S. et al. Quantification of lentiviral vector copy numbers in individual hematopoietic colony-forming cells shows vector dose-dependent effects on the frequency and level of transduction. Gene Therapy 18, 479-487, doi:10.1038/gt.2010.163 (2011).
140. Batard, P., Jordan, M. & Wurm, F. Transfer of high copy number plasmid into mammalian cells by calcium phosphate transfection. Gene 270, 61-68 (2001).
141. Cohen, R. N., van der Aa, M. A. E. M., Macaraeg, N., Lee, A. P. & Szoka, F. C. Quantification of Plasmid DNA Copies in the Nucleus after Lipoplex and Polyplex Transfection. Journal of controlled release: official journal of the Controlled Release Society 135, 166-174, doi:10.1016/j.jconre1.2008.12.016 (2009).
142. Hamann, A., Nguyen, A. & Pannier, A. K. Nucleic acid delivery to mesenchymal stem cells: a review of nonviral methods and applications. J Biol Eng 13, 7, doi:10.1186/s13036-019-0140-0 (2019).
143. Ramamoorth, M. & Narvekar, A. Non viral vectors in gene therapy- an overview. J Clin Diagn Res 9, GE01-06, doi:10.7860/JCDR/2015/10443.5394 (2015).
144. Baek, K., Tu, C., Zoldan, J. & Suggs, L. J. Gene Transfection for Stem Cell Therapy. Current Stem Cell Reports 2, 52-61, doi:10.1007/s40778-016-0029-5 (2016).
145. Abdul Halim, N. S., Fakiruddin, K. S., Ali, S. A. & Yahaya, B. H. A comparative study of non-viral gene delivery techniques to human adipose-derived mesenchymal stem cell. Int J Mol Sci 15, 15044-15060, doi:10.3390/ijms150915044 (2014).
146. Ho, Y. K., Zhou, L. H., Tam, K. C. & Too, H. P. Enhanced non-viral gene delivery by coordinated endosomal release and inhibition of beta-tubulin deactylase. Nucleic acids research 45, e38, doi:10.1093/nar/gkw1143 (2017).
147. Zou, R., Zhou, K., Stephanopoulos, G. & Too, H. P. Combinatorial engineering of 1-deoxy-D-xylulose 5-phosphate pathway using cross-lapping in vitro assembly (CLIVA) method. PLoS One 8, e79557, doi:10.1371/journal.pone.0079557 (2013).
148. Dominici, M. et al. Minimal criteria for defining multipotent mesenchymal stromal cells. The International Society for Cellular Therapy position statement. Cytotherapy 8, 315-317, doi:10.1080/14653240600855905 (2006).
149. Swiech, K. et al. Transient transfection of serum-free suspension FMK 293 cell culture for efficient production of human rFVIII. BMC Biotechnology 11, 114, doi:10.1186/1472-6750-11-114 (2011).
150. McCall, J., Nicholson, L., Weidner, N. & Blesch, A. Optimization of adult sensory neuron electroporation to study mechanisms of neurite growth. Frontiers in Molecular Neuroscience 5, doi:10.3389/fnmo1.2012.00011 (2012).
151. Ho, Yoon K. et al. Enhanced transfection of a macromolecular lignin-based DNA complex with low cellular toxicity. Bioscience Reports 38, BSR20181021, doi:10.1042/B5R20181021 (2018).
152. Madeira, C. et al. Gene delivery to human bone marrow mesenchymal stem cells by microporation. Journal of biotechnology 151, 130-136, doi:10.1016/j.jbiotec.2010.11.002 (2011).
153. Bourbeau, D., Lavoie, G., Nalbantoglu, J. & Massie, B. Suicide gene therapy with an adenovirus expressing the fusion gene CD::UPRT in human glioblastomas: different sensitivities correlate with p53 status. J Gene Med 6, 1320-1332, doi:10.1002/jgm.611 (2004).
154. Marofi, F., Vahedi, G., Biglari, A., Esmaeilzadeh, A. & Athari, S. S. Mesenchymal Stromal/Stem Cells: A New Era in the Cell-Based Targeted Gene Therapy of Cancer. Frontiers in Immunology 8, 1770, doi:10.3389/fimmu.2017.01770 (2017).
155. Pulukuri, S. M. K., Gorantla, B., Dasari, V. R., Gondi, C. S. & Rao, J. S. Epigenetic Upregulation of Urokinase Plasminogen Activator Promotes the Tropism of Mesenchymal Stem Cells for Tumor Cells. Molecular cancer research: MCR 8, 1074-1083, doi:10.1158/1541-7786.MCR-09-0495 (2010).
156. Kim, E.-K. et al. Endogenous Gastric-Resident Mesenchymal Stem Cells Contribute to Formation of Cancer Stroma and Progression of Gastric Cancer. Korean Journal of Pathology 47, 507-518, doi:10.4132/KoreanJPathol.2013.47.6.507 (2013).
157. Park, G. T., Kim, S. U. & Choi, K. C. Anti-proliferative Effect of Engineered Neural Stem Cells Expressing Cytosine Deaminase and Interferon-beta against Lymph Node-Derived Metastatic Colorectal Adenocarcinoma in Cellular and Xenograft Mouse Models. Cancer research and treatment: official journal of Korean Cancer Association 49, 79-91, doi:10.4143/crt.2015.503 (2017).
158. Twitty, C. G. et al. Retroviral Replicating Vectors Deliver Cytosine Deaminase Leading to Targeted 5-Fluorouracil-Mediated Cytotoxicity in Multiple Human Cancer Types. Human gene therapy methods 27, 17-31, doi:10.1089/hgtb.2015.106 (2016).
159. Christodoulou, I. et al. Mesenchymal stem cells in preclinical cancer cytotherapy: a systematic review. Stem Cell Res Ther 9, 336, doi:10.1186/s13287-018-1078-8 (2018).
160. Meleshina, A. V. et al. Influence of mesenchymal stem cells on metastasis development in mice in vivo. Stem Cell Res Ther 6, 15, doi:10.1186/s13287-015-0003-7 (2015).
161. Li, G. C. et al. Mesenchymal stem cells seldomly fuse with hepatocellular carcinoma cells and are mainly distributed in the tumor stroma in mouse models. Oncol Rep 29, 713-719, doi:10.3892/or.2012.2174 (2013).
162. Karnoub, A. E. et al. Mesenchymal stem cells within tumour stroma promote breast cancer metastasis. Nature 449, 557-563, doi:10.1038/nature06188 (2007).
163. Zischek, C. et al. Targeting tumor stroma using engineered mesenchymal stem cells reduces the growth of pancreatic carcinoma. Ann Surg 250, 747-753, doi:10.1097/SLA.0b013e3181bd62d0 (2009).
164. Niess, H. et al. Selective targeting of genetically engineered mesenchymal stem cells to tumor stroma microenvironments using tissue-specific suicide gene expression suppresses growth of hepatocellular carcinoma. Ann Surg 254, 767-774; discussion 774-765, doi:10.1097/SLA.0b013e3182368c4f (2011).
165. Nishikawa, G. et al. Bone marrow-derived mesenchymal stem cells promote colorectal cancer progression via CCRS. Cell Death & Disease 10, 264, doi:10.1038/s41419-019-1508-2 (2019).
166. Christodoulou, I. et al. Mesenchymal stem cells in preclinical cancer cytotherapy: a systematic review. Stem Cell Research & Therapy 9, 336, doi:10.1186/s13287-018-1078-8 (2018).
167. Golchin, A., Rekabgardan, M., Taheri, R. A. & Nourani, M. R. Promotion of Cell-Based Therapy: Special Focus on the Cooperation of Mesenchymal Stem Cell Therapy and Gene Therapy for Clinical Trial Studies. Adv Exp Med Biol 1119, 103-118, doi:10.1007/5584_2018_256 (2018).
168. Lee, M. W. et al. Mesenchymal stem cells in suppression or progression of hematologic malignancy: current status and challenges. Leukemia 33, 597-611, doi:10.1038/s41375-018-0373-9 (2019).
169. Portnow, J. et al. Neural Stem Cell-Based Anticancer Gene Therapy: A First-in-Human Study in Recurrent High-Grade Glioma Patients. Clinical cancer research: an official journal of the American Association for Cancer Research 23, 2951-2960, doi:10.1158/1078-0432.Ccr-16-1518 (2017).
170. Mangraviti, A. et al. Non-virally engineered human adipose mesenchymal stem cells produce BMP4, target brain tumors, and extend survival. Biomaterials 100, 53-66, doi:10.1016/j.biomaterials.2016.05. 025 (2016).
171. Nouri, F. S., Wang, X. & Hatefi, A. Genetically engineered theranostic mesenchymal stem cells for the evaluation of the anticancer efficacy of enzyme/prodrug systems. Journal of controlled release: official journal of the Controlled Release Society 200, 179-187, doi:10.1016/j.jconre1.2015.01.003 (2015).
172. von Einem, J. C. et al. Treatment of advanced gastrointestinal cancer with genetically modified autologous mesenchymal stem cells: Results from the phase 1/2 TREAT-ME-1 trial. Int J Cancer, doi:10.1002/ijc.32230 (2019).
173. Kucerova, L. et al. Cytosine deaminase expressing human mesenchymal stem cells mediated tumour regression in melanoma bearing mice. J Gene Med 10, 1071-1082, doi:10.1002/jgm.1239 (2008).
174. Kucerova, L., Poturnajova, M., Tyciakova, S. & Matuskova, M. Increased proliferation and chemosensitivity of human mesenchymal stromal cells expressing fusion yeast cytosine deaminase. Stem cell research 8, 247-258, doi:10.1016/j.scr.2011.11.006 (2012).
175. Kwon, S. K., Kim, S. U., Song, J. J., Cho, C. G. & Park, S. W. Selective delivery of a therapeutic gene for treatment of head and neck squamous cell carcinoma using human neural stem cells. Clinical and experimental otorhinolaryngology 6, 176-183, doi:10.3342/ceo.2013.6.3.176 (2013).
176. Boussif, O., Zanta, M. A. & Behr, J. P. Optimized galenics improve in vitro gene transfer with cationic molecules up to 1000-fold. Gene Ther 3, 1074-1080 (1996).
177. Christodoulou, I. et al. Mesenchymal stem cells in preclinical cancer cytotherapy: a systematic review. Stem cell research & therapy 9, 336, doi:10.1186/s13287-018-1078-8 (2018).
178. Niess, H. et al. Treatment of advanced gastrointestinal tumors with genetically modified autologous mesenchymal stromal cells (TREAT-ME1): study protocol of a phase I/II clinical trial. BMC cancer 15, 237-237, doi:10.1186/s12885-015-1241-x (2015).
179. von Einem, J. C. et al. Treatment of advanced gastrointestinal cancer with genetically modified autologous mesenchymal stem cells: Results from the phase 1/2 TREAT-ME1 trial. International Journal of Cancer 0, doi:10.1002/ijc.32230 (2019).
180. Dominici, M. et al. Minimal criteria for defining multipotent mesenchymal stromal cells. The International Society for Cellular Therapy position statement. Cytotherapy 8, 315-317, doi:10.1080/14653240600855905 (2006).
181. Xie, Y. et al. The Human Glioblastoma Cell Culture Resource: Validated Cell Models Representing All Molecular Subtypes. EBioMedicine 2, 1351-1363, doi:10.1016/j.ebiom.2015.08.026 (2015).
182. Schweizer, M. T. et al. A Phase I Study to Assess the Safety and Cancer-Homing Ability of Allogeneic Bone Marrow-Derived Mesenchymal Stem Cells in Men with Localized Prostate Cancer. Stem cells translational medicine 8, 441-449, doi:10.1002/sctm.18-0230 (2019).
183. Ferrari, C. et al. Limiting cell aggregation during mesenchymal stem cell expansion on microcarriers. Biotechnology progress 28, 780-787, doi:10.1002/btpr.1527 (2012).
184. Takahashi, I., Sato, K., Mera, H., Wakitani, S. & Takagi, M. Effects of agitation rate on aggregation during beads-to-beads subcultivation of microcarrier culture of human mesenchymal stem cells. Cytotechnology 69, 503-509, doi:10.1007/s10616-016-9999-5 (2017).
185. Rawat, J. & Gadgil, M. Shear stress increases cytotoxicity and reduces transfection efficiency of liposomal gene delivery to CHO-S cells. Cytotechnology 68, 2529-2538, doi:10.1007/s10616-016-9974-1 (2016).

All references cited herein and elsewhere in the specification are herein incorporated by reference in their entireties.

1. A mesenchymal stem cell (MSC) transfected with a nucleic acid construct from which one or more functional genes are expressed, the MSC having a multipotent phenotype which is substantially unchanged by the transfection of the nucleic acid construct, and the MSC being free of virus-based transfection vehicle materials.

2. (canceled)

3. The MSC of claim 1, wherein the transfected MSC is transfected with an average of at least about 1000, at least about 2000, at least about 3000, at least about 4000, at least about 5000, at least about 6000, at least about 7000, at least about 8000, at least about 9000, or at least about 10000 copies of the nucleic acid construct.

4. The MSC of claim 1, wherein the transfected MSC transiently expresses the one or more functional genes for at least about 7, at least about 8, at least about 9, at least about 10, at least about 11, at least about 12, at least about 13, at least about 14, at least about 15, at least about 16, or at least about 17 days following transfection.

5. The MSC of claim 1, wherein the transfected MSC is transfected with the nucleic acid construct using a cationic polymer, a first agent capable of redirecting endocytosed nucleic acids from intracellular acidic compartments, and a second agent capable of stabilizing a microtubular network of the MSC.

6. The MSC of claim 1, wherein the one or more functional genes comprise a suicide gene.

7. The MSC of claim 1, wherein the one or more functional genes comprise Cytosine Deaminase (CDy), uracil phosphoribosyltransferase (UPRT), or both.

8. The MSC of claim 1, wherein the multipotent phenotype includes tumor and/or cancer tropism properties of the MSC.

9. A method of treating cancer in a subject, the method comprising administering to the subject the MSC of claim 1, wherein the cancer is, for example lymphoma, clear cell carcinoma, glioblastoma, temozolomide resistant glioblastoma, perianal carcinoma, oral melanoma, thyroid carcinoma, soft tissue carcinoma, cancer ulceration, nasal tumor, or gastrointestinal cancer.

10. The method according to claim 9, wherein the method further comprises administering the subject 5FC, 5FU, GCV, or any combination thereof.

11. The MSC of claim 1, wherein the multipotent phenotype comprises an immunophenotype in which the expression of CD surface markers is substantially unchanged after transfection, preferably wherein the transfected MSC or MSCs are plastic-adherent, express CD105, CD73, and CD90 (>95%), lack expression of CD45, CD34, CD14, and HLA-DR surface molecules (<2%), and are capable of differentiating into osteoblasts, adipocytes, and chondroblasts in vitro, satisfying the immunophenotype criteria defined by the International Society for Cellular Therapy (ISCT).

12. A method for transfecting mesenchymal stem cells (MSCs) with a nucleic acid construct from which one or more functional genes are expressed, the method comprising:
exposing the MSCs to a transfection mixture comprising the nucleic acid construct which is complexed with a cationic polymer;
exposing the MSCs to a first agent capable of redirecting endocytosed nucleic acids from intracellular acidic compartments and a second agent capable of stabilizing a microtubular network of the MSCs; and
incubating the MSCs;
thereby providing MSCs transfected with the nucleic acid construct.

13. The method of claim 12, wherein the method comprises one or more of the following: (a) a method wherein the MSCs are not centrifuged during exposure to the transfection mixture, to the first agent and second agent, during incubation, or any combination thereof; (b) a method wherein the step of incubating the MSCs comprises incubating the MSCs for about 2 hours to about 48 hours; (c) a method wherein the step of exposing the MSCs to the first and second agents comprises replacing the transfection mixture with cell culture media supplemented with the first agent and second agent; (d) a method wherein the one or more functional genes comprise a suicide gene; (e) a method wherein the one or more functional genes comprise Cytosine Deaminase (CDy), uracil phosphoribosyltransferase (UPRT), or both; (f) a method wherein the transfected MSCs are each transfected with an average of at least about 1000, at least about 2000, at least about 3000, at least about 4000, at least about 5000, at least about 6000, at least about 7000, at least about 8000, at least about 9000, or at least about 10000 copies of the nucleic acid construct; and (g) a method wherein a multipotent phenotype of the transfected MSCs is substantially unchanged by the transfection.

14.-19. (canceled)

20. The method of claim 13, wherein the multipotent phenotype is one or more of the following: (a) a multipotent phenotype comprising tumor and/or cancer tropism properties of the MSC; and (b) a multipotent phenotype comprising an immunophenotype in which the expression of CD surface markers is substantially unchanged after transfection, preferably wherein the transfected MSCs are plastic-adherent, express CD105, CD73, and CD90 (>95%), lack expression of CD45, CD34, CD14, and HLA-DR surface molecules (<2%), and are capable of differentiating into osteoblasts, adipocytes, and chondroblasts in vitro, satisfying the immunophenotype criteria defined by the International Society for Cellular Therapy (ISCT).

21. (canceled)

22. A pharmaceutical composition comprising the MSC of claim 1, and at least one of a pharmaceutically acceptable carrier, diluent, excipient, cell media, or buffer.

23. A kit for transfecting a mesenchymal stem cell (MSC) with a nucleic acid construct from which one or more functional genes are transiently expressed, the kit comprising one or more of:
an MSC;
a nucleic acid construct designed for transient expression of one or more functional genes;
a cell culture media;
a cationic polymer;
a first agent capable of redirecting endocytosed nucleic acids from intracellular acidic compartments;
a second agent capable of stabilizing a microtubular network of the MSC;
instructions for performing a method as defined in claim 12;
5FC;
GCV; and/or
5FU.

24. The method of claim 12, wherein the step of exposing the MSCs to the transfection mixture comprises adding the transfection mixture to the MSCs without removing a growth medium from the MSCs, and centrifugation is not performed during the steps of exposing and incubating.

25. The method of claim 12, wherein the step of exposing the MSCs to the first agent and the second agent comprises adding the first and second agent to the MSCs simultaneously, sequentially, or in combination with the transfection mixture.

26. The method of claim 25, wherein the first and second agent are added to the MSCs simultaneously with addition of the transfection mixture to the MSCs, or wherein the first and second agent are mixed with the transfection mixture and added to the MSCs, or wherein the first and second agent are added to the MSCs shortly after the transfection mixture is added to the MSCs.

27. (canceled)

28. The method of claim 26, wherein the transfection mixture is not removed before the first and second agents are added to the MSCs.

29. The method of claim 12, wherein a duration of exposure of the MSCs to the transfection mixture overlaps with a duration of exposure of the MSCs to the first and second agents, preferably wherein the transfection mixture is not removed before the first and second agents are added to the MSCs.

30. (canceled)
